SEQUENCE LISTING (123 sequences)

SEQ ID NO: 1 - DNA, 148 bp, Homo sapiens
  source 1..148: mol_type = genomic DNA
gcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaag

SEQ ID NO: 2 - RNA, 24 nt, Homo sapiens
  source 1..24: mol_type = genomic RNA
tgttcagcttctgttagccactga

SEQ ID NO: 3 - RNA, 176 nt, Homo sapiens
  source 1..176: mol_type = genomic RNA
gaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagag

SEQ ID NO: 4 - RNA, 50 nt, Homo sapiens
  source 1..50: mol_type = genomic RNA
taaaaagacatggggcttcatttttgttttgcctttttggtatcttacag

SEQ ID NO: 5-11 - skipped (7 entries)

SEQ ID NO: 12 - protein, 7 aa, Homo sapiens
AKALAGQ

SEQ ID NO: 13 - protein, 7 aa, Homo sapiens
FCWLLLQ

SEQ ID NO: 14-16 - skipped (3 entries)

SEQ ID NO: 17 - RNA, 25 nt, synthetic construct
  source 1..25: mol_type = other RNA
attttataagcaagtaaacattaaa

SEQ ID NO: 18 - RNA, 25 nt, synthetic construct
  source 1..25: mol_type = other RNA
tttaatgtttacttgcttataaaat

SEQ ID NO: 19 - RNA, 25 nt, synthetic construct
  source 1..25: mol_type = other RNA
attaaatttagccaccctctggctg

SEQ ID NO: 20 - RNA, 25 nt, synthetic construct
  source 1..25: mol_type = other RNA
cagccagagggtggctaaatttaat

SEQ ID NO: 21 - RNA, 25 nt, synthetic construct
  source 1..25: mol_type = other RNA
gatttttaaaaagtacttcaataaa

SEQ ID NO: 22 - RNA, 25 nt, synthetic construct
  source 1..25: mol_type = other RNA
tttattgaagtactttttaaaaatc

SEQ ID NO: 23 - RNA, 25 nt, synthetic construct
  source 1..25: mol_type = other RNA
aataaattacatacttgtaattaat

SEQ ID NO: 24 - RNA, 25 nt, synthetic construct
  source 1..25: mol_type = other RNA
attaattacaagtatgtaatttatt

SEQ ID NO: 25-27 - skipped (3 entries)

SEQ ID NO: 28 - DNA, 23 bp, synthetic construct
  source 1..23: mol_type = other DNA
aatgggacatataaggtttttct

SEQ ID NO: 29 - DNA, 23 bp, synthetic construct
  source 1..23: mol_type = other DNA
aatgggacatataaggtttttct

SEQ ID NO: 30 - DNA, 18 bp, synthetic construct
  source 1..18: mol_type = other DNA
aagttgcttataaaatgt

SEQ ID NO: 31 - RNA, 26 nt, synthetic construct
  source 1..26: mol_type = other RNA
cattttataagcaagtaaacattaaa

SEQ ID NO: 32 - RNA, 26 nt, synthetic construct
  source 1..26: mol_type = other RNA
tttaatgtttacttgcttataaaatg

SEQ ID NO: 33 - RNA, 25 nt, synthetic construct
  source 1..25: mol_type = other RNA
attaaatttagccaccctctggctg

SEQ ID NO: 34 - RNA, 25 nt, synthetic construct
  source 1..25: mol_type = other RNA
cagccagagggtggctaaatttaat

SEQ ID NO: 35 - RNA, 25 nt, synthetic construct
  source 1..25: mol_type = other RNA
gatttttaaaaagtacttcaataaa

SEQ ID NO: 36 - RNA, 25 nt, synthetic construct
  source 1..25: mol_type = other RNA
tttattgaagtactttttaaaaatc

SEQ ID NO: 37 - RNA, 27 nt, synthetic construct
  source 1..27: mol_type = other RNA
aataaattacatacttgtaattaattg

SEQ ID NO: 38 - RNA, 27 nt, synthetic construct
  source 1..27: mol_type = other RNA
caattaattacaagtatgtaatttatt

SEQ ID NO: 39 - DNA, 25 bp, synthetic construct
  source 1..25: mol_type = other DNA
ggattctatacttctcaagtcattt

SEQ ID NO: 40 - DNA, 21 bp, synthetic construct
  source 1..21: mol_type = other DNA
aagagggatttttaaaaatct

SEQ ID NO: 41 - DNA, 19 bp, synthetic construct
  source 1..19: mol_type = other DNA
cgtttagagaacagtttct

SEQ ID NO: 42 - DNA, 19 bp, synthetic construct
  source 1..19: mol_type = other DNA
cgtttagagaacagattct

SEQ ID NO: 43 - DNA, 22 bp, synthetic construct
  source 1..22: mol_type = other DNA
gatgtcaaaagccctgtagtac

SEQ ID NO: 44 - DNA, 20 bp, synthetic construct
  source 1..20: mol_type = other DNA
tgtcaaaagccctgtagtac

SEQ ID NO: 45 - DNA, 21 bp, synthetic construct
  source 1..21: mol_type = other DNA
atgtcaaaagccctgtagtac

SEQ ID NO: 46 - DNA, 19 bp, synthetic construct
  source 1..19: mol_type = other DNA
gatggcatttctagtcttg

SEQ ID NO: 47 - DNA, 18 bp, synthetic construct
  source 1..18: mol_type = other DNA
  modified_base 4: mod_base = OTHER; note = pseudouridine
  modified_base 7: mod_base = OTHER; note = pseudouridine
aaancanaggggaagatg

SEQ ID NO: 48 - DNA, 24 bp, synthetic construct
  source 1..24: mol_type = other DNA
aaaaaggttcagcttctgttagcc

SEQ ID NO: 49 - DNA, 24 bp, synthetic construct
  source 1..24: mol_type = other DNA
aaaggtgaagcttctgttagccac

SEQ ID NO: 50 - DNA, 24 bp, synthetic construct
  source 1..24: mol_type = other DNA
  modified_base 3: mod_base = OTHER; note = pseudouridine
  modified_base 9: mod_base = OTHER; note = pseudouridine
aantaagantatgttagccactga

SEQ ID NO: 51 - DNA, 24 bp, synthetic construct
  source 1..24: mol_type = other DNA
aaagattctgttagccactgatta

SEQ ID NO: 52 - DNA, 22 bp, synthetic construct
  source 1..22: mol_type = other DNA
aagggccgtcctggagttcctg

SEQ ID NO: 53 - DNA, 22 bp, synthetic construct
  source 1..22: mol_type = other DNA
aagggccatcctggagttcctg

SEQ ID NO: 54 - DNA, 23 bp, synthetic construct
  source 1..23: mol_type = other DNA
  modified_base 4: mod_base = OTHER; note = pseudouridine
aaanggaaatcctggagttcctg

SEQ ID NO: 55 - DNA, 22 bp, synthetic construct
  source 1..22: mol_type = other DNA
aagggccgtcctggagttcctg

SEQ ID NO: 56 - DNA, 22 bp, synthetic construct
  source 1..22: mol_type = other DNA
aagggccatcctggagttcctg

SEQ ID NO: 57 - DNA, 25 bp, synthetic construct
  source 1..25: mol_type = other DNA
cccagccagagggtggctaaatttt

SEQ ID NO: 58 - DNA, 22 bp, synthetic construct
  source 1..22: mol_type = other DNA
aaaacggaggaaatacaattag

SEQ ID NO: 59 - RNA, 47 nt, synthetic construct
  source 1..47: mol_type = other RNA
gggctgtttttctcgctgactttcagccccaaacaaaaaagtcagca

SEQ ID NO: 60 - RNA, 29 nt, synthetic construct
  source 1..29: mol_type = other RNA
gggctgtttttctcgctgactttcagccc

SEQ ID NO: 61 - RNA, 13 nt, synthetic construct
  source 1..13: mol_type = other RNA
aaaaaagtcagca

SEQ ID NO: 62 - RNA, 10 nt, synthetic construct
  source 1..10: mol_type = other RNA
gggctgtttt

SEQ ID NO: 63 - RNA, 33 nt, synthetic construct
  source 1..33: mol_type = other RNA
gctgactttcagccccaaacaaaaaagtcagca

SEQ ID NO: 64 - RNA, 39 nt, synthetic construct
  source 1..39: mol_type = other RNA
tctccaaactagaaatgccatcttccttgatgttggagg

SEQ ID NO: 65 - DNA, 30 bp, synthetic construct
  source 1..30: mol_type = other DNA
ctccaacatgaaggaagatggcatttctag

SEQ ID NO: 66 - RNA, 18 nt, synthetic construct
  source 1..18: mol_type = other RNA
gatggcatttctaggctt

SEQ ID NO: 67 - RNA, 17 nt, synthetic construct
  source 1..17: mol_type = other RNA
  modified_base 3: mod_base = OTHER; note = pseudouridine
  modified_base 6: mod_base = OTHER; note = pseudouridine
aancanaggggaagatg

SEQ ID NO: 68 - RNA, 52 nt, synthetic construct
  source 1..52: mol_type = other RNA
gatttcttgtagatctgttctctaaacgaactttaaaatctatgtggctgtc

SEQ ID NO: 69 - DNA, 21 bp, synthetic construct
  source 1..21: mol_type = other DNA
taaagttcgtttagagaacag

SEQ ID NO: 70 - DNA, 18 bp, synthetic construct
  source 1..18: mol_type = other DNA
cgtttagagaacagtttc

SEQ ID NO: 71 - DNA, 15 bp, synthetic construct
  source 1..15: mol_type = other DNA
aagtgtaaagttcgt

SEQ ID NO: 72 - RNA, 18 nt, synthetic construct
  source 1..18: mol_type = other RNA
cgtttagagaacagtttc

SEQ ID NO: 73 - RNA, 15 nt, synthetic construct
  source 1..15: mol_type = other RNA
  modified_base 4: mod_base = OTHER; note = pseudouridine
  modified_base 6: mod_base = OTHER; note = pseudouridine
  modified_base 11: mod_base = OTHER; note = pseudouridine
  modified_base 12: mod_base = OTHER; note = pseudouridine
aagngnaaagnncgt

SEQ ID NO: 74 - RNA, 93 nt, synthetic construct
  source 1..93: mol_type = other RNA
gtttttaaacgggtttgcggtgtaagtgcagcccgtcttacaccgtgcggcacaggcactagtactgatgtcgtatacagggcttttgacatc

SEQ ID NO: 75 - RNA, 22 nt, synthetic construct
  source 1..22: mol_type = other RNA
gatgtcaaaagccctgtagtac

SEQ ID NO: 76 - RNA, 52 nt, synthetic construct
  source 1..52: mol_type = other RNA
gatctcttgtagatctgttctctaaacgaactttaaaatctatgtggctgtc

SEQ ID NO: 77 - RNA, 19 nt, synthetic construct
  source 1..19: mol_type = other RNA
cgtttagagaacagtttct

SEQ ID NO: 78 - RNA, 52 nt, synthetic construct
  source 1..52: mol_type = other RNA
gatctcttgtagatctgttctctaaacgaactttaaaatctgtgtggctgtc

SEQ ID NO: 79 - RNA, 32 nt, synthetic construct
  source 1..32: mol_type = other RNA
gctgactttcagccccaaacaaaaaagtcagc

SEQ ID NO: 80 - RNA, 29 nt, synthetic construct
  source 1..29: mol_type = other RNA
gctgactttcagcccgcgagggctgtttt

SEQ ID NO: 81 - RNA, 31 nt, synthetic construct
  source 1..31: mol_type = other RNA
aaaaaagtcagcgcgagctgactttcagccc

SEQ ID NO: 82 - RNA, 29 nt, synthetic construct
  source 1..29: mol_type = other RNA
gctgggtttttctctctttttatcccagc

SEQ ID NO: 83 - RNA, 10 nt, synthetic construct
  source 1..10: mol_type = other RNA
aaaaaaaaga

SEQ ID NO: 84 - RNA, 11 nt, synthetic construct
  source 1..11: mol_type = other RNA
gctgggttttt

SEQ ID NO: 85 - RNA, 32 nt, synthetic construct
  source 1..32: mol_type = other RNA
gctgtttttatcccagcaaaaaaaaaaacagc

SEQ ID NO: 86 - RNA, 32 nt, synthetic construct
  source 1..32: mol_type = other RNA
tctttttatcccagcgcgagctgggtttttct

SEQ ID NO: 87 - RNA, 34 nt, synthetic construct
  source 1..34: mol_type = other RNA
aaaaaaaaacagcgcgagctgtttttatcccagc

SEQ ID NO: 88 - RNA, 79 nt, unidentified
  source 1..79: mol_type = unassigned RNA
tgttttggctgggtttttccttgttcgcaccggacacctccagtgaccagacggcaaggtttttatcccagtgtatatt

SEQ ID NO: 89 - RNA, 10 nt, unidentified
  source 1..10: mol_type = unassigned RNA
aaaaaaaaaa

SEQ ID NO: 90 - RNA, 40 nt, unidentified
  source 1..40: mol_type = unassigned RNA
ggctgggtttttccttcgaaagaaggtttttatcccagtc

SEQ ID NO: 91 - RNA, 65 nt, Homo sapiens
  source 1..65: mol_type = genomic RNA
gctgggtttttccttgttcgcaccggacacctccagtgaccagacggcaaggtttttatcccagt

SEQ ID NO: 92 - RNA, 18 nt, Homo sapiens
  source 1..18: mol_type = genomic RNA
aaaaaaaaaaaaaaaaaa

SEQ ID NO: 93 - RNA, 93 nt, Homo sapiens
  source 1..93: mol_type = genomic RNA
gaaggtttttctyttcctgagaaaacaacacgtattgttttctcaggttttgctttttggcctttttctagcttaaaaaaaaaaaaagcaaaa

SEQ ID NO: 94 - RNA, 76 nt, synthetic construct
  source 1..76: mol_type = other RNA
ggaaggtttttctyttcctgaggcgaaagtctcaggttttgctttttggcctttcttaaaaaaaaaaaaagcaaaa

SEQ ID NO: 95 - RNA, 18 nt, synthetic construct
  source 1..18: mol_type = other RNA
aatattctgtggtgtcat

SEQ ID NO: 96 - RNA, 28 nt, synthetic construct
  source 1..28: mol_type = other RNA
gatctcttgtagatctgttctctaaacg

SEQ ID NO: 97 - DNA, 19 bp, synthetic construct
  source 1..19: mol_type = other DNA
  modified_base 12: mod_base = OTHER; note = uracil
gatggcatttctagtgttg

SEQ ID NO: 98 - RNA, 62 nt, synthetic construct
  source 1..62: mol_type = other RNA
tcagtggctaacagaagctgaacagtttctcagaaatacacaaattcctgagaattgggaac

SEQ ID NO: 99 - RNA, 57 nt, synthetic construct
  source 1..57: mol_type = other RNA
ggctaacagaagctgaacagtttctcagaaatacacaaattcctgagaattgggaac

SEQ ID NO: 100 - DNA, 22 bp, synthetic construct
  source 1..22: mol_type = other DNA
aaaggtgaagcttctgttagcc

SEQ ID NO: 101 - RNA, 49 nt, synthetic construct
  source 1..49: mol_type = other RNA
tcagtggctaacagaagctgaacagtttcgaaattgtatttatcttcag

SEQ ID NO: 102 - DNA, 21 bp, synthetic construct
  source 1..21: mol_type = other DNA
aaagattctgttagccactga

SEQ ID NO: 103 - RNA, 26 nt, synthetic construct
  source 1..26: mol_type = other RNA
ccaatgccatcctggagttcctgtaa

SEQ ID NO: 104 - DNA, 22 bp, synthetic construct
  source 1..22: mol_type = other DNA
caatgccatcctggagttcctg

SEQ ID NO: 105 - RNA, 58 nt, synthetic construct
  source 1..58: mol_type = other RNA
  modified_base 8: mod_base = OTHER; note = thymine
caggaactccaggatggcattgggcagcggcaaactgttgtcagaacattgaatgcaa

SEQ ID NO: 106 - DNA, 25 bp, synthetic construct
  source 1..25: mol_type = other DNA
  modified_base 3: mod_base = OTHER; note = pseudouridine
  modified_base 7: mod_base = OTHER; note = pseudouridine
aanaggngccatcctggagttcctg

SEQ ID NO: 107 - DNA, 24 bp, synthetic construct
  source 1..24: mol_type = other DNA
  modified_base 6: mod_base = OTHER; note = pseudouridine
aaaggngccatcctggagttcctg

SEQ ID NO: 108 - RNA, 23 nt, synthetic construct
  source 1..23: mol_type = other RNA
tttcttaagtatttgatgttaac

SEQ ID NO: 109 - RNA, 33 nt, synthetic construct
  source 1..33: mol_type = other RNA
gtaaacattaaatttagccaccctctggctggg

SEQ ID NO: 110 - RNA, 32 nt, synthetic construct
  source 1..32: mol_type = other RNA
ctaattgtatttcttaagtatttgatgttaac

SEQ ID NO: 111 - RNA, 51 nt, synthetic construct
  source 1..51: mol_type = other RNA
atttgatgttaaccttatatgtaacattttataagcaagtaaacattaaat

SEQ ID NO: 112 - DNA, 21 bp, synthetic construct
  source 1..21: mol_type = other DNA
aaaacttgttgtttcagttgg

SEQ ID NO: 113 - RNA, 15 nt, synthetic construct
  source 1..15: mol_type = other RNA
ctttgacgaaaatat

SEQ ID NO: 114 - RNA, 67 nt, synthetic construct
  source 1..67: mol_type = other RNA
agatttttaaaaagtacttcaataaattacatacttgtaattaattgacttgagaagtatagaatcc

SEQ ID NO: 115 - RNA, 60 nt, synthetic construct
  source 1..60: mol_type = other RNA
agatttttaaaaagtacttcaataaattacatacttgtaattaattgacttgagaagtat

SEQ ID NO: 116 - DNA, 21 bp, synthetic construct
  source 1..21: mol_type = other DNA
  modified_base 9: mod_base = OTHER; note = pseudouridine
  modified_base 10: mod_base = OTHER; note = pseudouridine
  modified_base 13: mod_base = OTHER; note = pseudouridine
aagaggganngtnaaaaatct

SEQ ID NO: 117 - RNA, 69 nt, synthetic construct
  source 1..69: mol_type = other RNA
taaagatatttaatcagtggctaacagaagctgaacagtttctcagaaagacacaaattcctgagaatt

SEQ ID NO: 118 - RNA, 124 nt, synthetic construct
  source 1..124: mol_type = other RNA
tcagtggctaacagaagctgaacagtttctcagaaagacacaaattcctgagaattgggaacatgctaaatacaaatggtatcttaaggtaagtctttgatttgttttttcgaaattgtattta

SEQ ID NO: 119 - RNA, 110 nt, synthetic construct
  source 1..110: mol_type = other RNA
ccaactgaaacaacaagttggcactgtagaatattgaggaaaagatggtcttattgcaaagattttcaataagaccatcctttcctcaatattctgtggtgtcatctttg

SEQ ID NO: 120 - DNA, 24 bp, synthetic construct
  source 1..24: mol_type = other DNA
aaaccaaattgttgtttcagttgg

SEQ ID NO: 121 - RNA, 35 nt, synthetic construct
  source 1..35: mol_type = other RNA
ccaactgaaacaacaagttggcactgtagaatatt

SEQ ID NO: 122 - RNA, 23 nt, synthetic construct
  source 1..23: mol_type = other RNA
aatattctgtggtgtcatctttg

SEQ ID NO: 123 - RNA, 13 nt, synthetic construct
  source 1..13: mol_type = other RNA
acgaaaatattag

(19) United States
(12) Patent Application Publication
(10) Pub. No.: US 2024/0327449 A1
(43) Pub. Date: Oct. 3, 2024

(54) STRUCTURE-BASED DESIGN OF ANTISENSE OLIGONUCLEOTIDE DRUGS

(71) Applicant: The Regents of the University of California, Oakland, CA (US)

(72) Inventors: Feng Guo, Los Angeles, CA (US); Vaithilingaraja Arumugaswami, Woodland Hills, CA (US)

(73) Assignee: The Regents of the University of California, Oakland, CA (US)

(21) Appl. No.: 18/579,766

(22) PCT Filed: Jul. 22, 2022
(86) PCT No.: PCT/US22/38037

§ 371 Date: Jan. 16, 2024

Related U.S. Application Data
(60) Provisional application No. 63/226,617, filed on Jul. 28, 2021.

(51) Int. Cl. C07H 21/04; C12N 15/113

(52) U.S. Cl. CPC C07H 21/04; C12N 15/113; C12N 2310/11; C12N 2310/315; C12N 2310/321; C12N 2310/322; C12N 2310/3233

(57) ABSTRACT

This invention provides a structure-based design method for making antisense oligonucleotides (ASOs) and ASOs made by this method. ASOs are an emerging class of drugs that are especially suitable for fighting a wide range of diseases. They are single-stranded synthetic oligonucleotides that specifically bind target RNAs and elicit desired biological and therapeutic effects. Conventional ASO design strategies do not adequately address this problem. The instant invention includes structure-based ASO designs that target RNAs critical in a variety of diseases.

### CROSS-REFERENCE TO RELATED APPLICATIONS

This application claims the benefit under 35 U.S.C. Section 119(e) of co-pending and commonly-assigned U.S. Provisional Patent Application Ser. No. 63/226,617, filed on Jul. 28, 2021, and entitled “STRUCTURE-BASED DESIGN OF ANTISENSE OLIGONUCLEOTIDE DRUGS” which application is incorporated by reference herein.

### STATEMENT REGARDING FEDERALLY SPONSORED RESEARCH AND DEVELOPMENT

This invention was made with government support under Grant Number 1616265, awarded by the National Science Foundation. The government has certain rights in the invention.

### TECHNICAL FIELD

The field of the invention relates to antisense oligonucleotide compositions and methods for making and using them.

### BACKGROUND OF THE INVENTION

Antisense oligonucleotides (ASOs) are single-stranded synthetic oligonucleotides that specifically bind target RNAs and elicit desired biological and therapeutic effects. Artisans in this field of technology have designed antisense, triplex and other oligonucleotide compositions which are capable of modulating expression of genes implicated in viral, fungal and metabolic diseases, and a number of antisense oligonucleotides have been approved for use as therapeutic agents in the treatment of disease states in animals and man. For example, in 1998, the phosphorothioate oligonucleotide drug, Vitravene (ISIS 2922), was approved by the FDA for treatment of cytomegalovirus retinitis in AIDS patients. While antisense oligonucleotides have been safely administered to humans for years, certain challenges in oligonucleotide design methodologies have limited the ability of these molecules to reach their full therapeutic potential.

ASOs designed to specifically bind to target RNAs and elicit desired biological and therapeutic effects are highly desirable. Unfortunately, RNA molecules tend to form complex secondary structures, which interferes with ASO binding to desired targets with appropriate affinity and specificity, thereby creating problems in this technical field.

There is a need for improved methods for making antisense oligonucleotides as well as improved antisense oligonucleotides made by such methods. For example, new technologies for making ASOs that can bind three-dimensional RNA targets that are associated with various disease states will open up new opportunities for therapeutic intervention, especially for genes that produce proteins previously deemed to be undruggable.

### SUMMARY OF THE INVENTION

As noted above, antisense oligonucleotides are an emerging class of drugs that are suitable for fighting a wide range of diseases. The invention disclosed herein is centered on a new structure-based design methodology for making such antisense oligonucleotides. Embodiments of the invention disclosed herein include methods that take into account the three-dimensional architectures of target RNA structures in ASO design strategies. Such structure-based methods can be used to make new ASOs targeting RNAs that are critical to a variety of disease states. In this context, the instant disclosure includes selection methods that have been discovered to be surprisingly useful for designing such unconventional ASOs. For example, the methods disclosed herein can be used to design ASOs comprising nucleotide base triples, Hoogsteen pairings and the like that are isosteric to the templates and enhance ASO/target interactions. The utility and importance of such tertiary interactions in ASO target binding is further demonstrated herein via biochemical analyses of illustrative ASO compositions of the invention.

The process for making 3D-ASOs disclosed herein typically includes a number of steps including first identifying specific RNA sequences to target. These target sequences include but not limited to exons, mRNA splice sites, intronic regions important for splicing, programmed ribosome frameshifting elements, microRNA-binding sites, protein-binding sites, polyadenylation signals, translation initiation sites, and other functionally important sites in 5′ and 3′ UTRs. In this context, the three dimensional structures of target sequences can be identified using RNA secondary structure prediction programs such as RNAstructure¹⁶ and MFOLD¹⁷. RNA secondary structure prediction programs most often provide a series of structures with low free energy levels. Usually, desirable target structures with low free energy levels are identified with high frequency using RNA secondary structure prediction programs. For example, RNA loop structures are considered highly desirable targets for the 3D-ASO designs disclosed herein. In contrast, relatively weak or low-confidence free energy structures identified by RNA secondary structure prediction programs can be ignored in the ASO design methodology disclosed herein. In addition, the structure of a target RNA does not have to be the same in the free and ASO-bound states. Sometimes the goal is to disrupt a native target RNA structure. In such situations, 3D-ASOs disclosed herein can act as modulators of the three-dimensional structures of naturally occurring ribonucleotide molecules, for example by perturbing a part of the structure and/or forming an alternative structure for such naturally occurring ribonucleotide molecules.

The disclosure provided herein describes a novel design methodology and further presents a series of illustrative 3D-ASO products made by such methods. In certain illustrative working embodiments, this disclosure provides data showing the strong inhibition of certain 3D-ASO designs against SARS-CoV-2 viral replication in human cells by targeting SARS-CoV-2 RNAs. This data demonstrates the power and unique value of the structural design methodologies disclosed herein, as well as ASOs made by these methods.

Detailed aspects of the methods disclosed herein are discussed below. Typically in the methods of the invention, once an appropriate hairpin or duplex structure has been identified as an RNA target sequence in a naturally occurring RNA molecule, specific nucleotide selection steps are taken to design 3D-ASO sequences that bind this target. For example, as discussed below, a design template can be used to select nucleotides to be disposed on the 5′- and/or 3′-end of such ASO sequences that can form non-canonical base pairings with naturally occurring RNA molecules, whereas other internal nucleotides in the ASO are selected for their sequence complementarity to a target oligonucleotide sequence in such RNA molecules. When two RNA structures are appropriately distanced within a target naturally occurring RNA molecule, both ends of an ASO may be modified to facilitate binding, for example using the 3D templates disclosed herein. For example, in illustrative templates A-D that are shown in FIG. 13, the strongest constraint comes from the Hoogsteen pair bridging the major-groove and minor-groove segments. Therefore, a first step in designing 3D-ASOs based on these templates is to determine if a nucleotide that forms a Hoogsteen pair with a target can be engineered in an ASO designed to target a specific RNA. If yes, the design can be focused on one or both ends of the ASO (e.g., the first 5 nucleotides at the 5′ and of the ASO and the last 5 nucleotides at the 3′ end of the ASO) in order to optimize the major-groove and minor-groove interactions. If no, one can alternatively either explore dynamic variants of the target RNA structures or apply one or more templates E-H, which do not require a Hoogsteen pair to bridge the major- and minor-groove segments. In templates A, C, E, and G, the 5′-end ASO residues recognize the targets' helical structures from their minor grooves but offer limited specificity to their sequences. Therefore, one can typically dedicate 2-3 3D-ASO residues for interacting (i.e., forming a binding interaction) a major or minor-groove triple segment. Moreover, properties of selected nucleotides such as those shown in FIG. 16 can be used to select the 3D-ASO nucleotides/sequences for interacting with major and minor groove segments of a target oligonucleotide sequence. In templates B, D, F, and H, the 3′ ASO residues recognize the targets' helical structures from their major grooves as well as the sequences via the major-groove base triples.

As noted above, in typical embodiments of the invention, nucleotides are selected and disposed in an ASO in order to form both non-canonical and canonical base pairings with a target sequence, with, for example, non-canonical base pairings typically being designed to occur in the 1-5 nucleotides present in the 3′ and 5′ terminal end(s) of the ASOs, and canonical base pairings typically being designed to occur in the other nucleotides of these ASOs. Such non-canonical and canonical base pairing combinations makes it possible to design new 3D-ASOs that bind to target sequences in naturally occurring RNA molecules with unexpectedly low free-energy profiles. The disclosure below pertaining to ASOs designed to target SARS-CoV-2 RNAs demonstrates the power and unique value of the structural design methodologies disclosed herein, as well as ASOs made by these methods.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A-1D provide cartoon schematics showing how ASOs interact with hairpins from a pseudoknot. FIG. 1(A) and FIG. 1(C) may be viewed as the hairpin-ASO pairs shown in FIG. 1(B) and FIG. 1(D) respectively. FIG. 1(A) and FIG. 1(C) represents the same pseudoknot.

FIGS. 2A-2E provides schematics showing the P2b-P3 pseudoknot structure of human telomerase RNA (PDB ID: 1YMO). The pseudoknot can be viewed as 2 pairs of hairpin-ASO interactions, FIG. 2(A) Hairpin1-ASO1 and FIG. 2(B) Hairpin2-ASO2. In panels FIG. 2A and FIG. 2B, secondary structure drawings of the pseudoknot are shown on the left and the 3D structures are on the right. Residues that can be omitted to turn the pseudoknot into a hairpin-ASO complex are shown in silver. Dashed blue lines indicate tertiary interactions detailed in panels FIG. 2C, FIG. 2D, and FIG. 2E. FIG. 2(C) Minor groove base triples involving A35 and A36 (most easily seen in the 3D structure of panel FIG. 2A). FIG. 2(D) A Hoogsteen base pair bridging the two base triple segments. FIG. 2(E) Major-groove base triples (most easily seen in panel FIG. 2B).

FIGS. 3A-3D provide schematics and data from affinity measurements showing the importance of tertiary interactions. FIG. 3(A) ASO1-Hairpin1 and FIG. 3(B) ASO2-Hairpin2 constructs. Truncations and mutations of the ASOs and hairpins are marked along with their Kd±standard error obtained from fitting. FIG. 3(C,D) ITC binding measurements for WT ASO1-Hairpin1 and ASO2-Hairpin2.

FIG. 4 provides schematics of ASO backbone modifications that can be used with embodiments of the invention.

FIGS. 5A-5N provide schematics of structure-based designs to improve eteplirsen. FIG. 5(A) The eteplirsen sequence is shown in black. It hybridizes to exon-51 residues that along with nearly sequences fold to a hairpin structure (green). The hairpin structure is incompatible with eteplirsen binding and will reduce its affinity. FIG. 5(B) the design DMD1 binds to the same hairpin. Instead of disrupting the hairpin, it forms extensive base triples and non-canonical base pairs (represented by dashed lines) with it, as shown in panel FIG. 5C-G. FIG. 5(H) The second design DMD2 binds to the other side of the hairpin loop and engages in tertiary interactions shown in panel FIG. 5I-N. ASO residues are colored dark red. ψ represents pseudouridine, the most abundant naturally occurring nucleotide.

FIGS. 6A-6R provide schematics of structure-based design of ASOs against SARS-CoV-2 RNA (See also Li et al., Structure-based design of antisense oligonucleotides that inhibit SARS-CoV-2 replication. bioRxiv (Preprint). 2021 Aug. 24:2021.08.23.457434. doi: 10.1101/2021.08.23.457434. PMID: 34462746; PMCID: PMC8404888; that is incorporated herein by reference. FIG. 6(A) A previous study identified a PMO ASO (TRS2) that effectively inhibits SARS-CoV by binding to the transcription regulatory sequence. FIG. 6(B) A structure-based design (SBD1) using PMO backbone. FIG. 6(C-G) Designed tertiary interactions between SBD1 and the target. FIG. 6(I) Design of a second PMO SBD2 that hybridizes to the 3′ region of the hairpin loop. FIG. 6(J-O) Expected target interactions for SBD2. “C+” represents cytosine in a protonated state. FIG. 6(Q,R) SBD3 and SBD4/SBD5, which are similar to SBD1 and SBD2 respectively, but with PS and 2′-OMe backbone. They can engage in additional based triples shown in panels H and P. ψ represents pseudouridine. T and U bases only differ from each other by a methyl group and are largely interchangeable in these designs. The choice can be made by commercial availability.

FIGS. 7A-7C provide schematics of 3D-ASO designs targeting the SARS-CoV-2 FSE. FIG. 7(A) The pseudoknot structure that causes ribosome stalling at the slippery site. FIG. 7(B) PRF3pPMO (with PMO backbone) is designed to disrupts stems 1 and 2. FIG. 7(C) A gapmer variant of PRF3pPMO.

FIGS. 8A-8H provide schematics of designs of 3D-ASOs targeting the SARS-CoV-2 TRS. FIG. 8(A) SBD1 with PMO backbone. TRS core residues are gray. FIG. 8(B-F) Designed tertiary interactions between SBD1 and viral RNA. FIG. 8(G) SBD3 is similar to SBD1, but with PS and 2′-OMe backbone. FIG. 8(H) Residue 13 of SBD3 can form a minor-groove base triple. T and U bases differ from each other by a methyl group and are largely interchangeable in such designs.

FIGS. 9A-9F provide data from the identification of lead 3D-ASO inhibitors using a cellular SARS-CoV-2 infection assay based on HEK293 cells expressing hACE2 receptor. Data were obtained at 24 hpi. FIG. 9(A) ICC images of infected cells stained for spike antigen at 10× magnification. A rabbit monoclonal antibody against SARS-CoV-2 Spike S1 protein was used. The scale bars represent 200 μm. FIG. 9(B) Quantification of spike-positive cells. The data shown are average±SD. FIG. 9(C) qRT-PCR quantification of viral RNA. Standard primer set amplifies the N-coding sequence. The data were normalized using host GAPDH mRNA. Shown are average #SD from three biological repeats. FIG. 9(D, E) Dose-dependent inhibition by PRF3p and SBD1, respectively, along with their variants. FIG. 9(F) Scatter plots of MTT cell viability assays. Horizontal bars indicate means and error bars represent SD.

FIGS. 10A-10C provide schematics of designs of FIG. 10(A) A 2D design in which the ASO is fully complementary to the whole hairpin loop of the target RNA. FIG. 10(B) 3D model of the ASO-loop duplex and the hairpin stem are incompatible. FIG. 10(C) An ASO called PREV, which was previously reported to inhibit SARS-CoV, one which represents a spatially incompatible design.

FIGS. 11A-11E provide schematics inspired from the P2b-P3 pseudoknot structure of human telomerase RNA (PDB ID: 1YMO). The pseudoknot can be viewed as two pairs of hairpin-ASO interactions: FIG. 11(A) Hairpin 1-ASO1 and FIG. 11(B) Hairpin 2-ASO2. In panels A and B, secondary structure drawings of the pseudoknot are shown on the left and the 3D structures are on the right. Residues that can be omitted to turn the pseudoknot into a hairpin-ASO complex are shown in silver. Dashed blue lines indicate tertiary interactions detailed in panels FIG. 11C, FIG. 11D, and FIG. 11E. FIG. 11(C) Minor groove base triples involving A35 and A36 (most easily seen in the 3D structure of panel FIG. 11A). FIG. 11(D) A Hoogsteen base pair bridging the two base triple segments. FIG. 11(E) Major-groove base triples (most easily seen in panel FIG. 11B).

FIGS. 12A-12F provide data from affinity measurements show the importance of tertiary interactions. (FIG. 12A, FIG. 12B) ITC binding measurements for ASO1-Hairpin 1 and ASO2-Hairpin 2. (FIG. 12C, FIG. 12D, FIG. 12E, FIG. 12F) Truncations and mutations of the ASOs and hairpins decrease the affinity. Kd values are shown along with standard errors obtained from fitting.

FIGS. 13A-13H provide schematics of eight 3D-ASO design templates useful in embodiments of the invention with illustrative ASO structures coupled to the templates.

FIGS. 14A-14E provide schematics of the structure of the PAN RNA from Kaposi's sarcoma-associated herpesvirus. FIG. 14(A) Schematic of the PAN RNA, highlighting the interaction between the ENE and the poly(A) tail. FIG. 14(B,C) Crystal structure of the ENE core (in green) bound with an A9 oligonucleotide representing the poly(A) tail (in magenta). The residues in gray are engineered to facilitate crystallization. FIG. 14(D) Schematic representation of the structure, with major-groove triples indicated by Leontis-Westhof notation and the A-minor interactions by dotted lines. FIG. 14(E) The A-minor interactions have geometry different from those in the telomerase pseudoknot. See also, Mitton-Fry et al. Science 330:1244-1247 (2010).

FIGS. 15A-15D provide schematics of the structure of human lncRNA MALAT1. FIG. 15(A) Comparison of the PAN and MALAT1 pseudoknots. MALAT1 core is the engineered construct used for structure determination. FIG. 15(B) Crystal structure of MALAT1 core. FIG. 15(C) Major-groove base triples. FIG. 15(D) A-minor interactions. See also, Brown et al. Nat. Struct. Mol. Biol. 21:633-640 (2014).

FIGS. 16A-16Q provide schematics showing major-groove triples that are geometrically compatible with the 3D-ASO design templates. This table can be used to decide what residues to use in the β position whereas the residues at the α and γ positions are from the target RNAs. The triples involving non-canonical base pairs are highlighted in the box, whereas those including a modified base pseudouridine (w) are boxed with dashed lines. Panels G and H are alternative choices.

FIGS. 17A-17G provide schematics showing major-major-groove triple table that can be used to decide what residues to use at the a position (labeled in red) whereas the residues at the β and γ positions are from the target RNAs (labeled in green). FIG. 17(A-D) Triples with pyrimidine residues at the α position. These triples are isosteric to the structural templates and therefore are preferred. FIG. 17(E-G) Triples with purine residues at the α position may also be used.

FIGS. 18A-18H provide schematics showing Hoogsteen pairs that can bridge the major-groove and minor-groove base triple segments.

FIGS. 19A-19D provide schematics of a summary of minor-groove base triples used to decide ASO residues at the a position in template A and C.

FIGS. 20A-20F provide schematics of a summary of minor-groove base triples used to decide ASO residues at the B position in template FIG. 20B and FIG. 20D. ASO residues often contain chemically modified ribose. There are several modifications to select from. This fact is highlighted by blue circles in the figure around unmodified ribose moieties. The modifications compatible with the minor-groove triples remain to be determined experimentally. It is believed that interactions shown in panels FIG. 20E and FIG. 20F are viable.

FIGS. 21A-21G provide schematics of 3D-ASO designs targeting the SARS-CoV-2 FSE. FIG. 21(a) Schematic of the genomic RNA of SARS-CoV-2. The transcription regulatory sequence (TRS) and frameshift stimulation element (FSE) are indicated by black horizontal lines. The 3D-ASOs targeting these sites are shown as thick red bars. FIG. 21(b) The pseudoknot structure in FSE that causes ribosome stalling at the slippery site (boxed). The UAA (SEQ ID NO: 5) stop codon for ORF1a is shown in black italic. FIG. 21(c) PRF3pPMO (an ASO with PMO backbone) is designed to disrupt stems 1 and 2, allowing the ribosome to proceed to the stop codon and fall off. Dashed lines represent tertiary interactions. FIG. 21(d) Hoogsteen pair between PRF3pPMO and FSE. FIG. 21(e-g) Major-groove base triples between PRF3pPMO and FSE base pairs in Stem 3.

FIGS. 22A-22L provide schematics pertaining to the design of SBD1 and SBD1-T15A targeting the SARS-CoV-2 TRS. FIG. 22(a) The previously reported ASO called TRS2 but named PREV here was proposed to bind to a hairpin loop. FIG. 22(b) SBD1 with PMO backbone. FIG. 22(c-h) Designed tertiary interactions between SBD1 and viral RNA. FIG. 22(i) Recent studies strongly suggest an alternative conformation for the 5′-UTR. The residues in the hairpin in panel b (colored in green) are involved in three stem-loop structures. FIG. 22(j) SBD1 has to disrupt SL3 but can engage in tertiary interactions with SL2, similar to those shown in panels FIG. 22(c), FIG. 22(d), and FIG. 22(f). FIG. 22(k) The SBD1-T15A variant optimizes the interaction with the alternative conformation. FIG. 22(l) SBD1-T15A engages with the single-hairpin TRS conformation by disrupting the U(5′−1)-A(3′+1) pair and at the same time forming A15-U(5′−1) WC pair and T16-A(3′+1) Hoogsteen pair. U(5′−1) and A(3′+1) are highlighted in a hollow font.

FIGS. 23A-23N provide schematics of 3D-designs that may be used to improve eteplirsen binding. FIG. 23(A) The eteplirsen sequence is shown in black. It hybridizes to exon-51 residues that along with nearly sequences fold to a hairpin structure (green). The hairpin structure is incompatible with eteplirsen binding and will reduce its affinity. FIG. 23(B) a designed DMD1 binds to the 5′ region of the hairpin loop. Instead of disrupting the hairpin, it forms base triples and non-canonical base pairs (represented by dashed lines) with it, as shown in panels FIG. 23C-E. FIG. 23(F) Our second design DMD2 binds to the 3′ region of the hairpin loop and engages in tertiary interactions shown in panels G-N. 3D-ASO residues are colored dark red. ϕ represents pseudouridine.

FIGS. 24A-24E provide schematics of 3D-ASO designs for skipping DMD exon 44.

FIGS. 25A-25F provide schematics of 3D-ASO designs for skipping DMD exon 45.

FIGS. 26A-26G provide schematics of structure-based designs of ASOs targeting alternative PAS sites of human FAN1. FIG. 26(A,B) ASOs targeting the 3′ and 5′ regions flanking the hairpin containing PAS1. FIG. 26(C,D) ASOs targeting the PAS1 hairpin loop. FIG. 26(E,F) ASOs targeting the hairpin containing PAS2. Dash lines represent tertiary interactions. Non-canonical pairs (shown as dots) are engineered in FAN1SBD2 and FAN1SBD6 to facilitate formation of base triples. ψ in FAN1SBD6 represents pseudouracil.

FIGS. 27A-27D provide schematics of loop structures in RNA molecules (FIGS. 27A-27C) and ASO studies using illustrative templates disclosed herein (FIG. 27D).

FIGS. 28A-28Q provide schematics of how chemically modified nucleobases can be used in 3D-ASO designs (see main text for detailed explanations). The chemical modifications are highlighted.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

As a new type of drug, ASOs are revolutionizing medicine^{1, 2}. ASO hybridizations do not rely on complementarity to protein-binding surfaces and in principle any sequences could be targeted following the Watson-Crick (WC) base pairing rule. Hence, ASO therapeutics can regulate cellular pathways that are not readily druggable with small molecules³. In 1978, Zamecnik and Stephenson demonstrated that ASOs can inhibit viral replication in vitro^{4, 5}. In 1998, the first ASO drug Fomivirsen received approval for treating cytomegalovirus (CMV) retinitis in immunocompromised patients. Although its market authorization was later withdrawn because the number of CMV infection cases was dramatically reduced by the development of HIV drugs, Fomivirsen demonstrated the feasibility and potential of ASOs as antivirals. Since 2013, seven ASO drugs gained approval to treat a variety of genetic disorders. To date, 66 ASO drug candidates are in clinical trials for a wide range of diseases^{6, 7}, including three for hepatitis B (Pharmaprojects database).

ASO therapeutics hybridize to target RNAs and elicit therapeutic effects via several mechanisms, such as inducing RNA degradation by cellular RNase H, inhibiting translation, causing exon inclusion or skipping during pre-mRNA splicing, competing with RNA-binding proteins and microRNAs for regulation, and inhibition of viral RNA replication, transcription, and viral particle assembly⁸. To achieve serum stability and favorable pharmacokinetics, ASO drugs are chemically modified to be resistant to the nucleases that are abundant in serum and cells. These chemical modifications must be compatible with the mechanism of drug action.

A class of ASOs contains DNA residues that hybridize with target RNAs and cause their degradation by cellular RNase H. The earliest ASOs such as Fomivirsen contained only a phosphorothioate (PS) modification in the backbone, which increases nuclease resistance but does not interfere with RNase H activity. The second-generation RNase H-dependent ASOs contain additional 2′-O-(2-methyloxyethyl) (2′-MOE) modifications in the 10 outermost residues of a 20mer oligo. These residues contribute to binding target RNAs but are not compatible with RNase H cleavage. By narrowing the window of RNase H cleavage to the central 10-nucleotide region, these “gapmers” have improved specificity⁹. Mipomersen, inotersen, and volanesorsen are gapmers. Another category of ASOs (such as eteplirsen, nusinersen, golodirsen, and viltolarsen) does not depend in RNase H but instead sterically blocks the targets from interacting with cellular or viral machinery. These “steric blockers” contain 2′-MOE, phosphorodiamidate morpholino (PMO), or other backbone modifications, which enhance both target-binding affinity and nuclease resistance^{2, 7}. The invention disclosed herein considers these mechanisms and chemical modifications in the methods disclosed herein for making novel 3D-ASO designs.

The invention disclosure herein has a number of embodiments. Embodiments of the invention include, for example, processes/methods for making an antisense oligonucleotide product. Typically, these methods first comprise selecting a ribonucleotide target sequence that is the target of the ASO. In this selection step, the ribonucleotide target sequence is selected as one present in a naturally occurring ribonucleotide molecule. Typically the ribonucleotide target sequence is from 8-30 nucleotides in length and comprises a segment of ribonucleotides that forms an RNA loop structure in the naturally occurring ribonucleotide molecule; or comprises a segment of ribonucleotides that are from 1 to 25 nucleotides 3′ or 5′ to a segment of ribonucleotides that forms a hairpin structure in the naturally occurring ribonucleotide molecule. In this methodology, a next step is constructing the antisense oligonucleotide product. Typically in the methods of the invention, the antisense oligonucleotide product is constructed by forming an antisense oligonucleotide product by selecting a plurality of nucleotides such that when covalently coupled together the plurality of nucleotides form an antisense oligonucleotide product having a segment of polynucleotides that is complementary to the ribonucleotide target sequence (as occurs with conventional ASOs). However, this methodology further includes selecting nucleotides for this ASO such that: (a) one or more nucleotides within the antisense oligonucleotide product are selected to form a bonding interaction with a major-groove RNA triplex structure or a minor-groove RNA triplex structure within the naturally occurring ribonucleotide molecule; and/or (b) one or more nucleotides within the antisense oligonucleotide product is selected to form a Watson Crick base pairing with (e.g. with a first nucleotide in) the ribonucleotide target sequence and a Hoogsteen base pairing with (e.g. with a second nucleotide in) the naturally occurring ribonucleotide molecule); and/or (c) nucleotides within the antisense oligonucleotide product do not form base pairing interactions with at least two proximal nucleotides in the ribonucleotide target sequence, wherein the at least two proximal nucleotides in the ribonucleotide target sequence that do not form base pairing interactions with the antisense oligonucleotide product are flanked by a plurality of nucleotides in the ribonucleotide target sequence that form Watson Crick base pairings with the antisense oligonucleotide product. Following the above-noted selection/determination steps, a next step in this methodology is making the antisense oligonucleotide product (e.g., using conventional polynucleotide synthesis techniques) from the plurality of selected nucleotides so that the antisense oligonucleotide product is made.

In certain embodiments of the invention, nucleotides selected for incorporation into the ASO product can be selected by considering the strength of various ASO nucleotide tertiary interactions with a target RNA structure, strengths which can be indicated by the free energy (ΔG) values (which can be calculated from dissociation constants (Kd)). For example, in certain embodiments of the invention, one or more nucleotides within the antisense oligonucleotide product can be selected to form a bonding interaction with a major-groove or minor groove RNA structure in the naturally occurring ribonucleotide molecule that contributes to the free energy of ASO binding (e.g. one that contributes at least 0.1 kcal/mole, 0.25 kcal/mole, 0.5 kcal/mole or 1 kcal/mole of free energy to binding). In certain embodiments of the invention, one or more nucleotides within the antisense oligonucleotide product can be selected to form a Hoogsteen base pairing with the naturally occurring ribonucleotide molecule that contributes to the free energy of ASO binding (e.g. one that contributes at least 0.1 kcal/mole, 0.25 kcal/mole, 0.5 kcal/mole or 1 kcal/mole of free energy to binding).

A wide variety of ASO products can be made using the methods disclosed herein. Typically, the antisense oligonucleotide product comprises between 13 and 30 nucleotides (e.g., from 17 to 25 nucleotides). In some embodiments of the invention, at least 5, 6, 7, 8, 9 or 10 nucleotides in the antisense oligonucleotide product are selected to form a polynucleotide segment in the antisense oligonucleotide product having 100% complementarity to the ribonucleotide target sequence; and the antisense oligonucleotide product comprises at least 1, 2, 3, 4, or 5 nucleotides that do not form Watson Crick bonds with the ribonucleotide target sequence such that the antisense oligonucleotide product is not 100% complementary to the ribonucleotide target sequence. In certain embodiments of the invention, the first five terminal 5′ and/or 3′ nucleotides in the antisense oligonucleotide are selected to comprise at least 1 nucleotide that forms: (i) a Hoogsteen base pairing; or (ii) a bonding interaction with a RNA major-groove; or (iii) a bonding interaction with a RNA minor-groove in the naturally occurring ribonucleotide molecule. In one illustrative embodiment of the invention, the antisense oligonucleotide product consists of from 13 to 30 nucleotides; wherein at least 5 nucleotides in the antisense oligonucleotide product are selected to form a polynucleotide segment in the antisense oligonucleotide product having 100% complementarity to the ribonucleotide target sequence; and terminal 5′ and/or 3′ 5 nucleotides in the antisense oligonucleotide product are selected to comprise at least 1 nucleotide that forms a bonding interaction with a major-groove RNA structure or a minor-groove RNA structure in the naturally occurring ribonucleotide molecule. In another illustrative embodiment of the invention, the antisense oligonucleotide product consists of from 17 to 25 nucleotides; wherein at least 10 nucleotides in the antisense oligonucleotide product are selected to form a polynucleotide segment in the antisense oligonucleotide product having 100% complementarity to the ribonucleotide target sequence; and at least 1 nucleotide in the antisense oligonucleotide forms a Watson Crick base pairing with the ribonucleotide target sequence and a Hoogsteen base pairing with the naturally occurring ribonucleotide molecule.

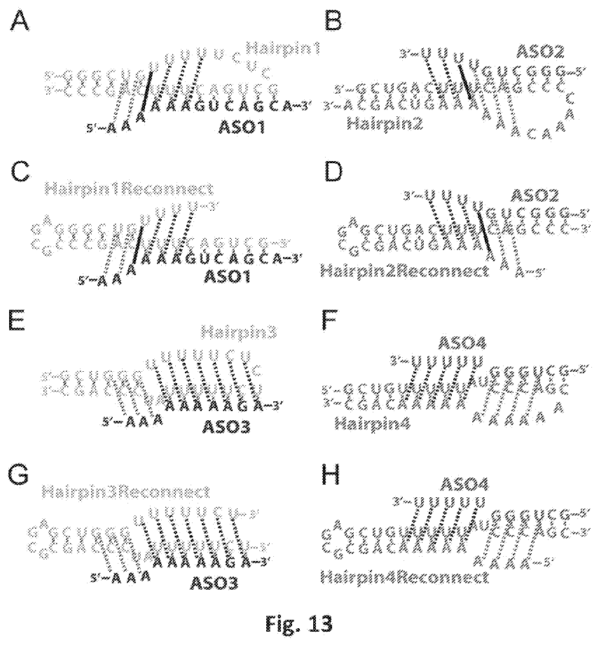

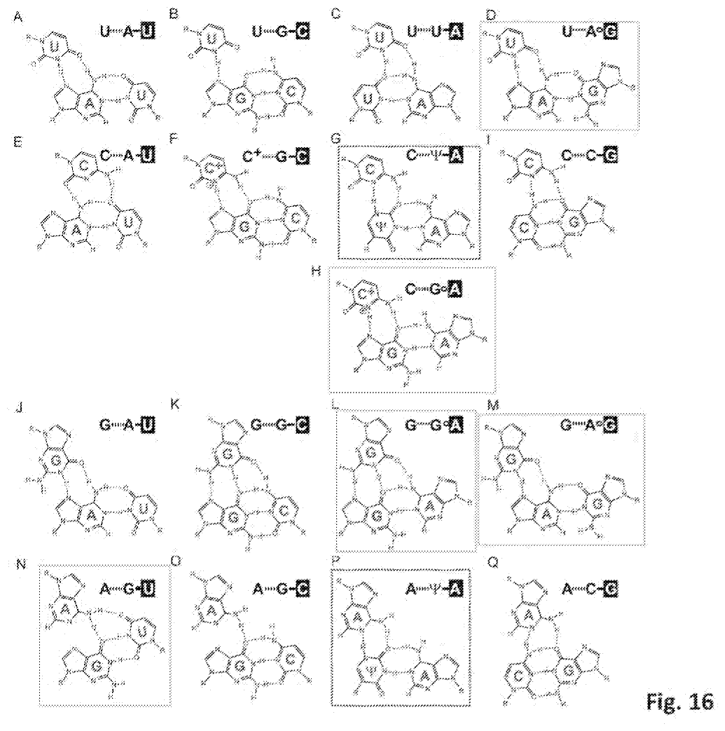

In certain embodiments of the invention, the process for making an ASO comprises examining antisense oligonucleotide product nucleotide and ribonucleotide target sequence nucleotide binding energies using a design template disclosed herein (e.g. as shown in FIG. 13) and/or the properties of nucleotide pairing interactions disclosed herein (e.g. as shown in FIG. 16). In some embodiments of the invention, the antisense oligonucleotide product comprises at least one of: a pseudouridine; a phosphorothioate linkage; a morpholino nucleotide; a modification of the 2′ sugar position of a ribose moiety; a 2′-MOE methoxyethyl moiety; a 2-fluoro moiety; or a 2′-hydroxy moiety.

Embodiments of the invention also include antisense oligonucleotides having the properties that occur when an ASO is made by a process disclosed herein. One such embodiment of the invention is an ASO product made by a process disclosed herein. Embodiments of the invention include, for example, antisense oligonucleotides having a segment of polynucleotides (e.g. at least 5-10 polynucleotides) that is complementary to a ribonucleotide target sequence (as occurs with conventional ASOs) as well as (a) one or more nucleotides within the antisense oligonucleotide that form a bonding interaction with a major-groove RNA structure or a minor-groove RNA structure within the naturally occurring ribonucleotide molecule; and/or (b) one or more nucleotides within the antisense oligonucleotide that form a Watson Crick base pairing with (e.g. with a first nucleotide in) the ribonucleotide target sequence and a Hoogsteen base pairing with (e.g. with a second nucleotide in) the naturally occurring ribonucleotide molecule); and/or (c) nucleotides within the antisense oligonucleotide are disposed in an arrangement such that they do not form continuous Watson-Crick base pairing interactions with at least two proximal nucleotides in the ribonucleotide target sequence, wherein the at least two proximal nucleotides in the ribonucleotide target sequence that do not form base pairing interactions with the antisense oligonucleotide are flanked by a plurality of nucleotides in the ribonucleotide target sequence that form Watson Crick base pairings with the antisense oligonucleotide.

In certain embodiments of the invention, the antisense oligonucleotide consists essentially of from 13 to 30 nucleotides; and at least 5 nucleotides in the antisense oligonucleotide form a polynucleotide segment in the antisense oligonucleotide having 100% complementarity to the ribonucleotide target sequence; and the first five terminal 5′ and/or 3′ nucleotides in the antisense oligonucleotide comprise at least 1 nucleotide that participate in a major-groove base triple interaction or in a minor-groove base triple interaction. In some embodiments of the invention, the antisense oligonucleotide consists of from 17 to 25 nucleotides; and at least 10 nucleotides in the antisense oligonucleotide form a polynucleotide segment in the antisense oligonucleotide having 100% complementarity to the ribonucleotide target sequence; and at least 1 nucleotide in the antisense oligonucleotide forms a Watson Crick base pairing with the ribonucleotide target sequence and a Hoogsteen base pairing with the naturally occurring ribonucleotide molecule. In certain embodiments of the invention, an ASO is designed to include certain selected nucleotides, linkages and the like. For example, optionally the antisense oligonucleotide comprises at least one of: a pseudouridine; a phosphorothioate linkage; a morpholino nucleotide; a modification of the 2′ sugar position of a ribose moiety; a 2′-MOE methoxyethyl moiety; a 2-fluoro moiety; or a 2′-hydroxy moiety.

Embodiments of the invention also include ASO compositions comprising at least one of: a pharmaceutical excipient, a liposome or a nanoparticle. For example, certain embodiments of the compositions of the invention include, for example a pharmaceutical excipient such as one selected from the group consisting of a preservative, a tonicity adjusting agent, a detergent, a viscosity adjusting agent, a sugar and a pH adjusting agent. For compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein.

The ASOs disclosed herein can be designed to target a wide variety of naturally occurring RNA molecules. In some embodiments of the invention, the target sequence in the naturally occurring RNA is present in a ribonucleotide expressed by a virus, a bacteria or a fungi. In certain embodiments of the invention, the target sequence in the naturally occurring RNA is present in a ribonucleotide expressed by a human cell. In certain embodiments of the invention, the target sequence in the naturally occurring RNA is present in a ribonucleotide expressed by a human parasite. In illustrative embodiments of the invention, a target sequence is present in a ribonucleotide expressed by a human tau gene; a human beta amyloid gene; a Covid 19 gene; a human Duchenne muscular dystrophy gene; a human FANCD2/FANC1-associated nuclease 1 (KIAA1018) gene; or a human microRNA (miRNA) gene.

In illustrative working embodiments of the invention, a target sequence is present in a ribonucleotide expressed by a Covid 19 virus and the ASO comprises or consists of a sequence: 5′-CGTTTAGAGAACAGTTTCT-3′(SEQ ID NO: 41); 5′-CGTTTAGAGAACAGATTCT-3′ (SEQ ID NO: 42); 5′-GATGTCAAAAGCCCTGTAGTAC-3′ (SEQ ID NO: 43); 5′-TGTCAAAAGCCCTGTAGTAC-3′(SEQ ID NO: 44); or 5′-ATGTCAAAAGCCCTGTAGTAC-3′(SEQ ID NO: 45).

As noted above, embodiments of the invention include methods that consider non-canonical tertiary binding interactions between one or more nucleotides in an ASO (e.g., nucleotides at the 5′ and 3′ ends of the ASO) and moieties on the naturally occurring RNA molecule (e.g. a major groove, a minor groove, nucleotides capable of forming an Hoogsteen pair and the like). In this context, the strength of major- and minor-groove base triples and Hoogsteen pair ASO/target binding interactions can be observed by measuring binding affinities using methods such as isothermal titration calorimetry. Aspects of this and the information discussed in the following sections are discussed in; Vieregg et al., Journal of the American Chemical Society. 2007; 129(48): 14966-73. Epub 2007 Nov. 13. doi: 10.1021/ja0748090. PubMed PMID: 17997555; PMCID: PMC2528546; Chen et al., Biochemistry. 2013; 52(42): 7477-85. Epub 2013 Oct. 9. doi: 10.1021/bi4008275. PubMed PMID: 24106785; PMCID: PMC3859436; Theimer et al., Mol Cell. 2005; 17(5):671-82. Epub 20May 3, 2008. doi: 10.1016/j.molcel.2005.01.017. PubMed PMID: 15749017; and Lee et al., Nucleic acids research. 1984; 12(16):6603-14. doi: 10.1093/nar/12.16.6603. PubMed PMID: 6473110; PMCID: PMC320099, the contents of which are incorporated by reference.

The strength of such tertiary interactions can be indicated by the free energy (ΔG) values, which can be calculated from dissociation constants (Kd). It is important to note that these values are to be determined in a solution close to intracellular pH and salt conditions (e.g., 20 mM HEPES pH 7.0, 100 mM KCl) so as to inform artisans how much a 3D-ASO module can enhance the affinity for its target RNA. Note that such physiological like conditions are very different from the “standard” high-salt condition (1M NaCl) the RNA base pairing free energy parameters are typically measured and derived. Reduced salt concentration and substituting Na with K both reduce the ΔG values of RNA hairpin and duplex formation (see, e.g., Vieregg et al., Journal of the American Chemical Society. 2007; 129(48): 14966-73. Epub 2007 Nov. 13. doi: 10.1021/ja0748090. PubMed PMID: 17997555; PMCID: PMC2528546; Chen et al., Biochemistry. 2013; 52(42):7477-85. Epub 2013 Oct. 9. doi: 10.1021/bi4008275. PubMed PMID: 24106785; PMCID: PMC3859436). Consequently, the ΔG values are smaller than those typically seen in RNA folding studies and in RNA secondary structure predictions. Typically, a 1.36 kcal/mol of free energy gain should enhance the affinity by 10 folds and 2.72 kcal/mol should enhance the affinity by 100 fold. The section below provide further guidance for artisans.

Free Energy Gains from Major-Groove Base Triples

Three layers of favorable major-groove base triples can contribute up to 2 kcal/mol of free energy to binding. The most favorable major-groove base triples include G●A-G (A-G represents a non-canonical base pair, with both bases hydrogen-bond using their Watson-Crick surfaces), U●A-U, U●A-G, C+●G-C (C+ represents a protonated C), G●G-A, and C+●G-A

Free Energy Gain from Engineering in Two Minor-Groove Base Triples

The contribution of two favorable minor-groove base triples is estimated to be 0.9 kcal/mol. The minor-groove triple interactions appear to tolerate a variety of base combinations, with the most favorable ones including G●G-C, C●G-C, A●G-C, C●A-U, A●A-U, and G●A-U.

Free Energy Gains from the Hoogsteen Pair

The contribution by the Hoogsteen pair cannot be measured alone because loss of this interaction is likely to disrupt neighboring major-groove and minor groove base triples. For example, when the U●A Hoogsteen pair (panel A in the figure immediately below) is replaced with C and G residues, which are unable to form a Hoogsteen pair at neutral pH (see, e.g. Theimer et al., Mol Cell. 2005; 17(5):671-82. Epub 2005 Mar. 8. doi: 10.1016/j.molcel.2005.01.017. PubMed PMID: 15749017), no association of ASO2 and Hairpin2 Reconnect can be detected. This result indicates loss of free energy for at least 2 kcal/mol. However, using the optimal U●A Hoogsteen pair as reference, G●G and G●A (panels B and C in the figure immediately below) have been determined to be viable alternatives, with free energy 0.22 and 0.40 kcal/mol higher than that of U●A, respectively. Chemically modified bases can mediate Hoogsteen pairing (examples shown in panels D-F in the figure immediately below, details explained in the section on chemical modifications) and thereby are useful in 3D-ASO designs.

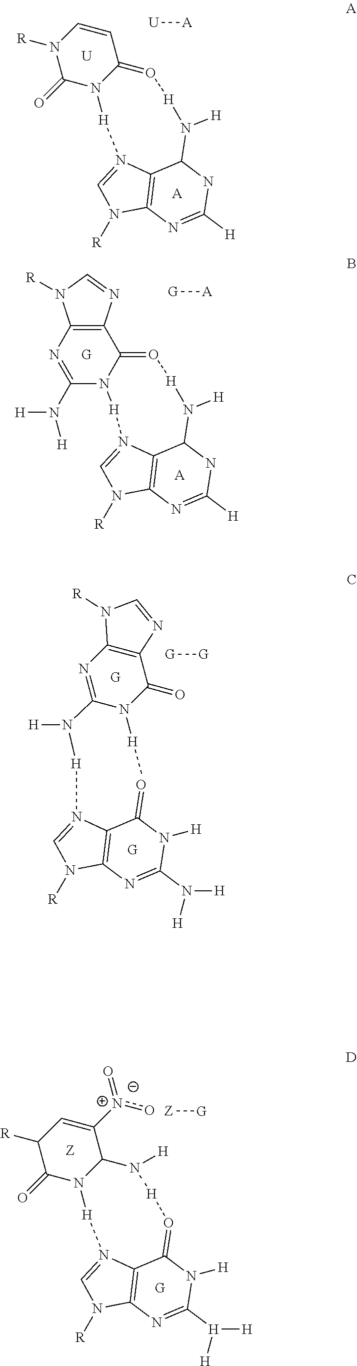

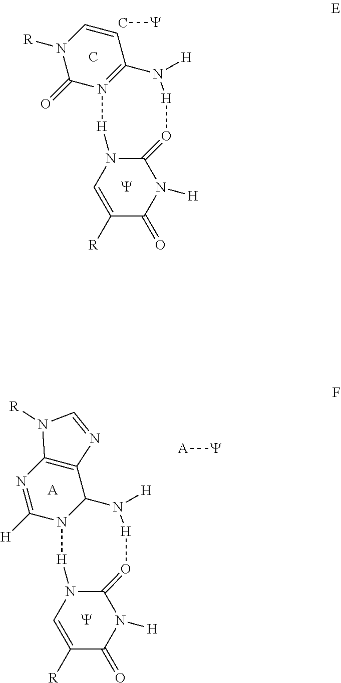

### Scoring Function for 3D-ASO Designs by Free Energy Gains

A detailed thermodynamic description of ASO-target interactions can facilitate scoring 3D-ASO designs. Namely, the total free energy gain can be calculated by summing up the free energy obtained from all the tertiary interactions engineered in a 3D-ASO design.

### Illustrative ASO Backbone Modifications

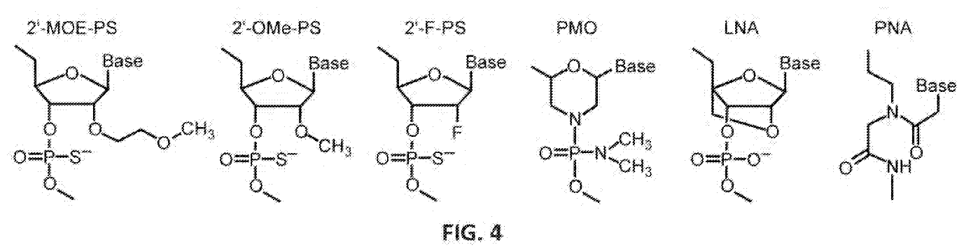

ASO backbone modifications useful in 3D-ASO designs include Phosphorothioate (PS), Phosphorodithioate (PS2), 2′-O-methoxyethyl (2′-MOE), 2′-O-methyl (2′-OMe), Locked nucleic acid (LNA), Peptide nucleic acid (PNA) (FIG. 4).

The phosphorodithioate linkage (PS2) is achiral, making it easier to study using structural biology methods. Previous studies have shown that DNA with PS2 linkages activates RNase H (useful for Gapmer designs) and that PS2 is completely resistant to hydrolysis by various nucleases.

### Illustrative ASO Base Modifications

### PseudoUridine (Ψ)

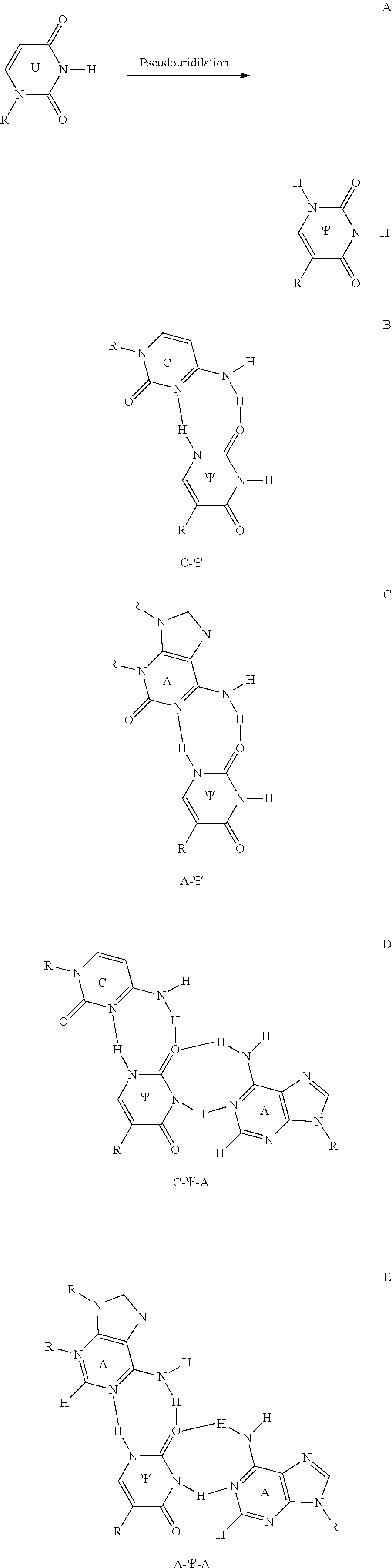

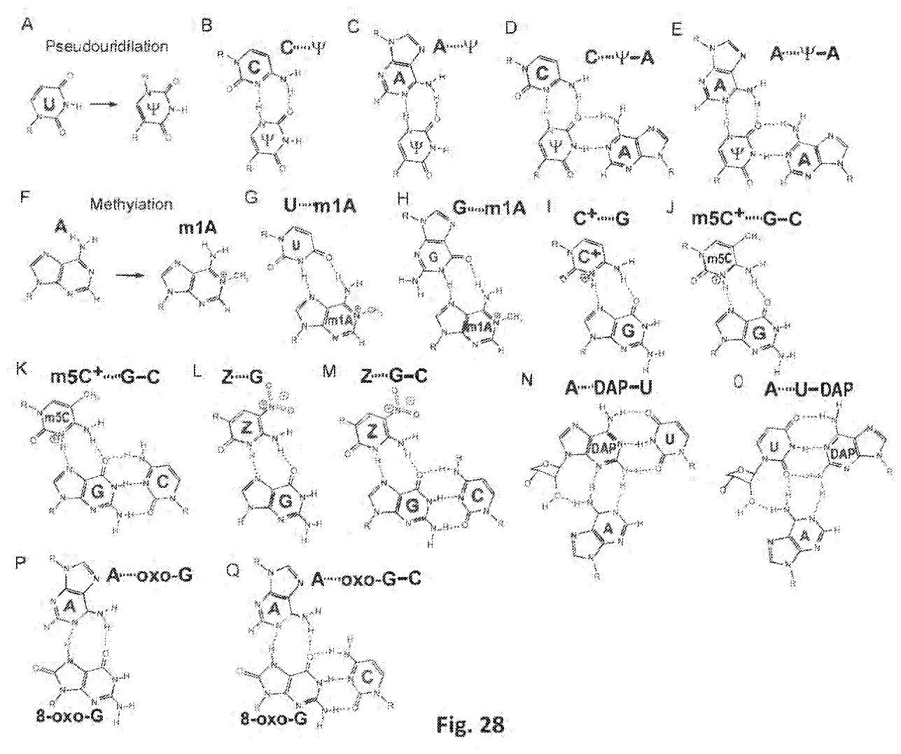

PseudoUridine is one of the most common modified nucleosides found in natural RNA. Compared to U, Y has increased a hydrogen bond donor group on the Hoogsteen surface (FIG. 28A). Therefore, it can form Hoogsteen pairs with either a C or A (FIGS. 28B and 28C) and can form major-groove base triples C⋅Ψ-A and A⋅Ψ-A (FIGS. 28 D and 28E).

### N1-methyladenosine (m1A)

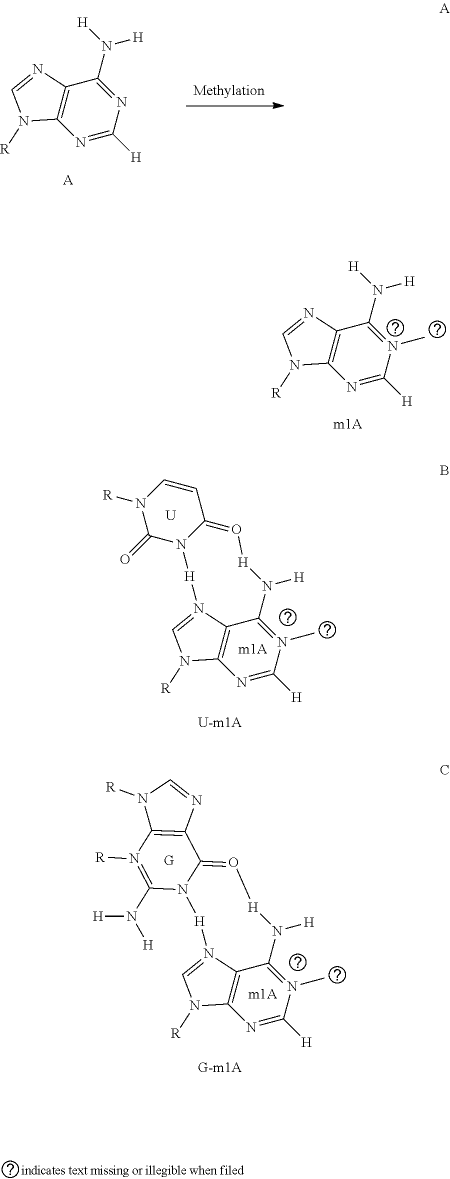

Methylation of adenosine at position 1 produces a drastic functional change in the nucleobase (FIG. 28F). 1-Methyladenosine (pKa 8.25) is a much stronger base than adenosine (pKa 3.5) and thus has a positive charge under physiological conditions. N−1 methylation maintains the ability to pair with U and G using its Hoogsteen surface (FIGS. 28G and 28H) but excludes potential pairing with non-specific targets via its Watson-Crick surface. Therefore, this modification is expected to enhance the specificity of 3D-ASOs.

### 5-Methyl-C (m5C)

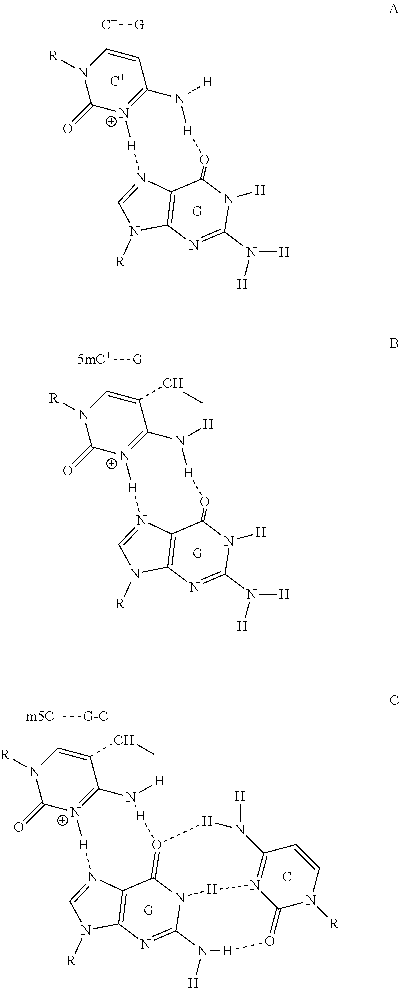

The formation of C●G Hoogsteen pair (FIG. 28I) requires the C to be protonated at the N3 position, which is represented as C⁺ in the diagram. However, the pKa of free C in solution is 4.5, making the protonation difficult under physiological pH conditions. This explains why the C●G combination is unfavorable at the Hoogsteen in our 3D-ASO design templates. The pKa of m5C is shifted to neutral pH and therefore is more ready to be protonated and form a Hoogsteen pair with G (FIG. 28J) (see, e.g. Chen et al., Biochemistry. 2013; 52(42): 7477-85. Epub 2013 Oct. 9. doi: 10.1021/bi4008275. PubMed PMID: 24106785; PMCID: PMC3859436). Additionally, formation of C●G-C major-groove base triple requires the major-groove C to be protonated, but in this chemical environment, the pKa of the C is shifted closer to the neutral pH. When incorporated in a 3D-ASO, m5C is expected to similarly stabilize the major-groove interaction with a G-C pair (FIG. 28K).

### Z Nucleotide

The requirement of C to be protonated in a C●G Hoogsteen pair (FIG. 28I) and a C●G-C major-groove base triple can be addressed using the Z nucleotide (6-amino-5-nitro-2(1H)-pyridone) (FIGS. 28L and 28M).

### 2,6-diaminopurine (DAP)

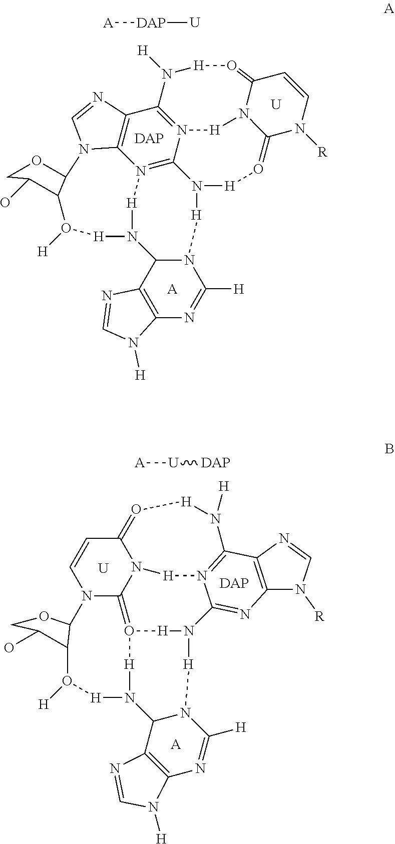

DAP is an A with an amino group at position 2. This amino group allows DAP to form three hydrogen bonds with U, strengthening the Watson-Crick pairing interaction (FIGS. 28N and 28O). More importantly, the amino group also strengthens minor-groove base triples by providing a hydrogen-bond donor.

### 8-Oxo-G

8-Oxo-G can use its Hoogsteen surface to pair with an A (FIG. 28P). In addition, it can form the major-groove triple A●Oxo-G-C (FIG. 28Q). These features are useful for 3D-ASO designs.

Further aspects and embodiments of the invention are discussed in the following sections.

### Further Principles of Structure-Based ASO Designs

Conventional ASO designs strictly follow the rule of Watson-Crick (WC) base pairing along linear target sequences. However, target RNAs often fold into strong secondary and tertiary structures that interfere with ASO hybridization. Single-stranded RNAs tend to form intramolecular base pairs, which stack with each other to form double-stranded helices and higher order structures. These structures are often important for biological functions such as recruiting RNA-binding proteins¹⁰. For example, viruses often contain RNA hairpins in their 5′- and 3′-UTRs¹¹, which are important for viral replication and translation, and in the ribosome frame shifting (RFS) sites responsible for continuing translation of downstream viral proteins. These structures often hinder ASO hybridization, as their pairing interactions and 3D geometries are not compatible with each other. Conventional ASO design strategies either avoid targeting structured regions or ignore these structures and rely on screening large numbers of ASOs to identify high-affinity binders. This challenge has resulted in the failure of many ASO candidates and leaves important structured targets as inaccessible.

The structure-based design methodology disclosed herein solves this problem by being compatible with target structures in three-dimensional (3D) space and engaging in tertiary interactions in addition to WC base pairing. The “3D-ASOs” designed this way bind targets with enhanced affinity due to the tertiary interactions gained and the free energy saved for not having to disrupt target structures. Moreover, binding specificity is elevated because 3D-ASOs not only recognize target sequences but also the shapes and hydrogen-bonding patterns.

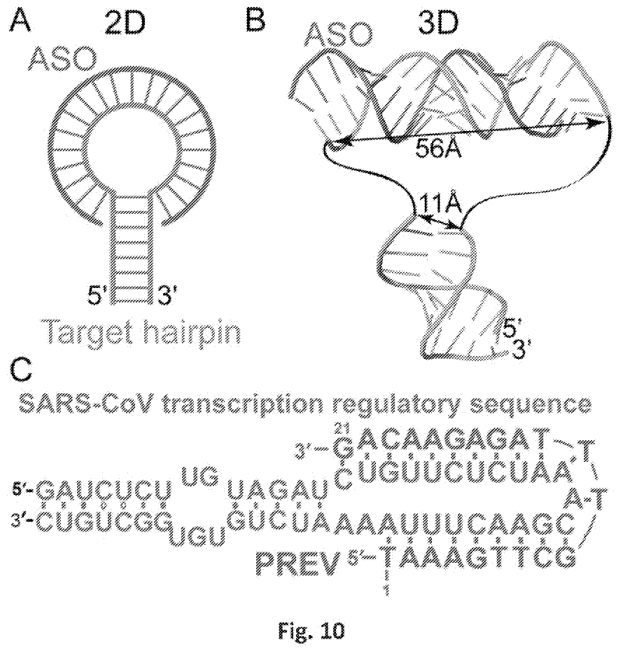

Structure is a critical consideration for successful drug discovery. However, tertiary interactions have not been incorporated in ASO drug development. This is in part due to traditional thinking of RNAs as a simple string of nucleotides. The invention disclosed herein introduces/considers aspects of structural biology to the field of ASO development. The importance of 3D thinking is highlighted in the example shown in FIG. 10. The practice of 1D design naturally leads to the desire to have ASOs complement the whole hairpin loop (FIG. 10A). However, not all base pairs will form due to 3D constraints. Base pairing in the hairpin stem requires the 5′- and 3′-most residues of the loop to be close (11 Å) to each other, whereas the helix formed between the hairpin loop and ASO is like a rigid rod and extends 56 Å for a 21-bp duplex (FIG. 10B). From this modeling exercise, it is concluded that it is unfavorable for ASOs to pair with the entire hairpin loop, a situation often seen in the literature (FIG. 10C for example).

### Structural Design Templates

To achieve 3D designs, eight innovative design templates for targeting a specific hairpin were generated. The design templates were inspired by existing RNA structures. These templates make sure that the ASO and target are spatially and structurally compatible and that, in addition to WC base pairing, they engage in extensive tertiary interactions. These tertiary interactions increase the ASO-target interface and thus enhance the affinity and specificity. Importantly, such design templates allow structured RNAs to be targeted. Because hairpins are the most fundamental and most abundant structures in RNAs, the instant technologies are broadly applicable to a wide range of targets and diseases.

A key feature of the 3D-ASOs disclosed herein is that they are not fully complementary to targets in sequences. This feature clearly sets this technology apart from the great deal of ASO drug development programs in both industry and academia. Therefore, this platform can be used to either improve existing ASOs or develop better ASOs de novo. Importantly, these 3D-ASOs are not fully complementary to target sequences and therefore have clear features that distinct them from the conventional designs.

Four Design Templates Derived from the Telomerase Pseudoknot Structure

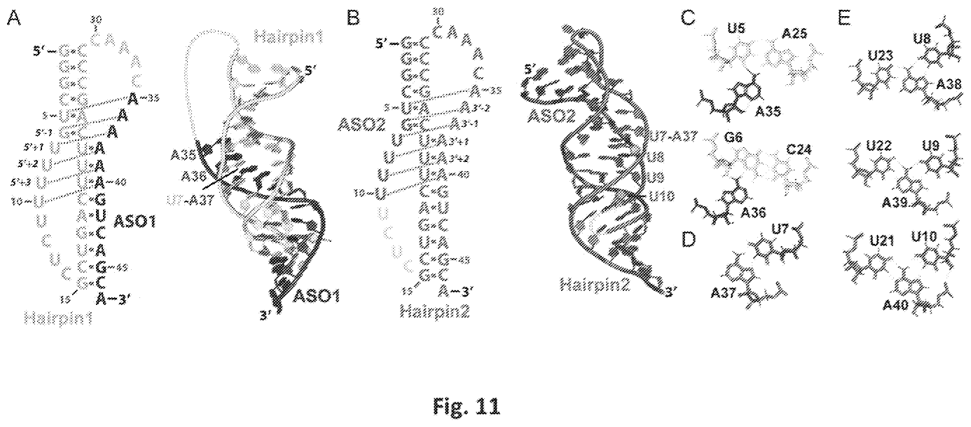

The invention disclosed herein is built upon an understanding of how an ASO and a structured target RNA interact. Unfortunately however, the required structural information is lacking, i.e., few 3D structures of RNA hairpins in complex with ASOs are available. Therefore, aspects of the invention start out by inspecting RNA structures, such as pseudoknots, that resemble the association between ASOs and hairpins. Pseudoknot refers to an RNA hairpin with its loop pairing with an upstream or downstream strand, which may be viewed as equivalent to an ASO (FIG. 11A, B). In fact, each pseudoknot minimally contains two paired regions. Either one may be viewed as the hairpin stem, whereas the other as the duplex formed between the hairpin loop and an ASO.

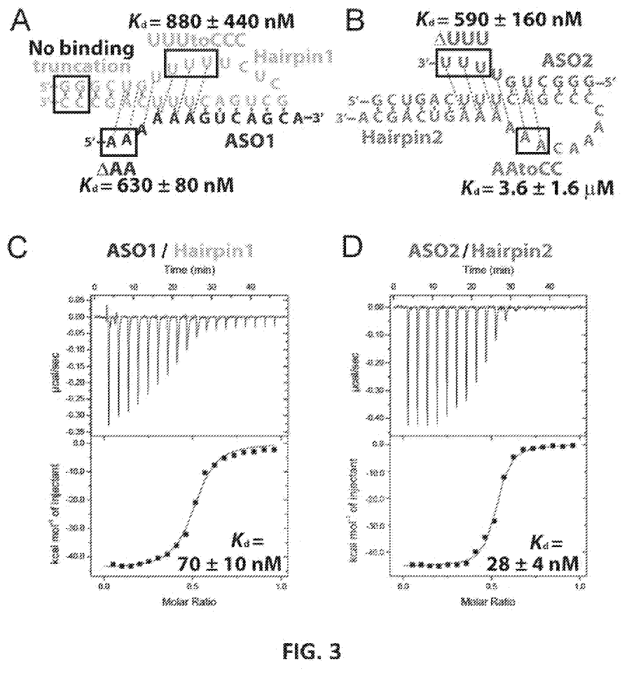

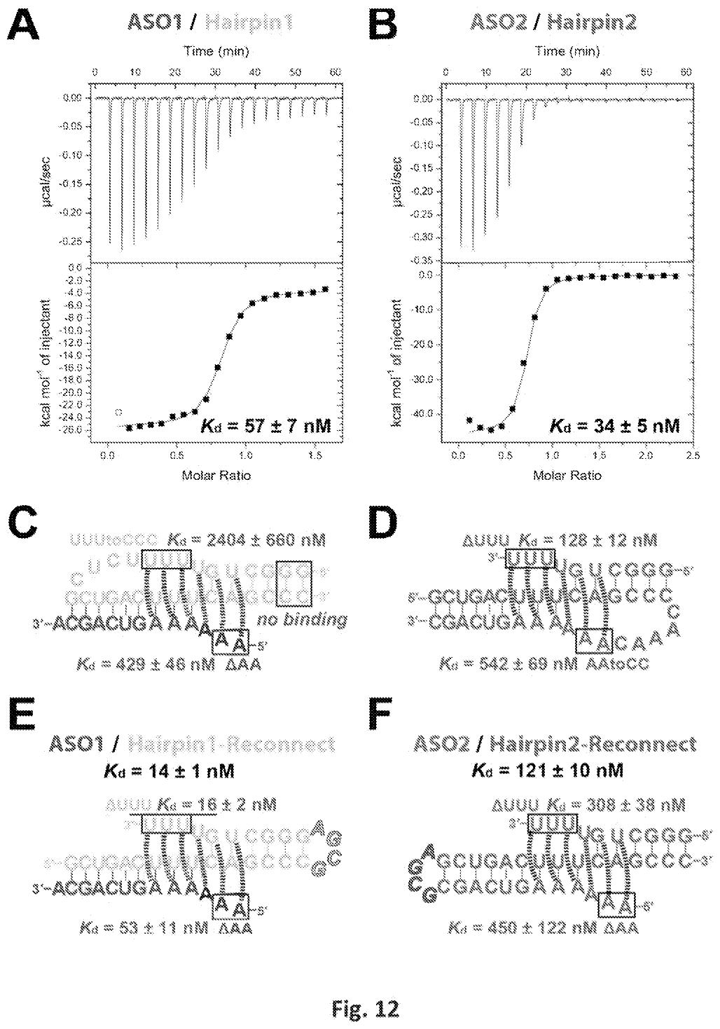

93 pseudoknot structures were examiner to observe extensive tertiary interactions, including major-groove and minor-groove base triples (a third base contacting a base pair from the major- or minor-groove side respectively, see FIGS. 3E and 3C for examples), non-WC base pairs, and base stacking. Among them, the telomerase RNA pseudoknot structure stands out as having the most extensive tertiary interactions. Several pseudoknot structures were selected to design ASO-hairpin pairs, and then test their binding. Indeed, the ASO-hairpin pairs derived from the telomerase pseudoknot showed high affinity binding (FIG. 12). Therefore, this structure was used as the template for subsequent biochemical characterization and ASO design work.

The telomerase RNA pseudoknot structure suggests two distinct ways ASOs can bind hairpin targets (FIG. 11A, B). ASO1 primarily pairs with 3′ region of the Hairpin 1 loop (FIG. 11A). Two A residues overhang at the 5′-end of ASO1 (A35 and A36 in pseudoknot numbering), contacting the base pairs in the hairpin stem from the minor groove side (FIG. 11C). Such interactions are called A-minor interactions, a type of tertiary interaction that is important for RNA folding and is widely observed in RNAs such as group I introns and ribosomal RNAs^{12, 13}. ASO2 primarily pairs with 5′ region of the Hairpin 2 loop (FIG. 11B). Three overhanging U residues at the 3′-end of ASO2 (U8-U10 in pseudoknot numbering) contacts the base pairs in the Hairpin 2 stem from the major groove side (FIG. 11E). Even though these ASO residues are not involved in WC pairing, they nevertheless engage in extensive hydrogen bonds and the consecutive layers of base triples stack with each other. These tertiary interactions should contribute to binding affinity and structural specificity. As only some base combinations are compatible with base triples, it is expected that these ASO residues also help recognize the target sequences they contact directly. Both types of “stem triples” are particularly useful for targeting hairpins with relatively short loops.

Two additional features are important for ASO design. First, both minor-groove and major-groove interactions are present in each ASO-hairpin pair. The minor-groove triples involving ASO1 5′ overhang residues (A35 and A36; FIG. 11A) occur between 3′ loop residues in Hairpin 2 and the duplex formed between ASO2 and Hairpin 2 loop (FIG. 11B). Similarly, the major-groove triples involving the 3′ overhang (U8-U10) of ASO2 (FIG. 11B) are used by 5′ loop residues of Hairpin 1 to engage the ASO1-Hairpin 1 loop duplex (FIG. 11A). The second feature is that a Hoogsteen pair (between U7 and A37, pseudoknot numbering) bridges the double-layers of minor-groove triples and the triple-layers of major-groove triples (FIG. 11D). This Hoogsteen pair is important for proper geometry connecting the two neighboring segments.

### Base Triple Interactions are Essential for High-Affinity Binding

The base triples in the ASO-hairpin pairs were disrupted and their affinities measured using isothermal titration calorimetry (ITC) (FIG. 12A-D). The K_{d} values are 70±10 nM for ASO1/Hairpin 1 and 28±4 nM for ASO2/Hairpin 2 (FIG. 12A, B). Deletion of the two 5′ A overhangs reduced ASO1 binding to Hairpin 1 by 8-fold, indicating the importance of the minor-groove triples (FIG. 12C). Mutation of the three U residues at the 5′ end of Hairpin 1 loop to Cs reduced binding by >10-fold, highlighting the critical contribution of the major-groove triples to ASO1-Hairpin 1 binding. Truncation of the three U overhanging residues in ASO2 reduced binding to Hairpin 2 by 20-fold (FIG. 12D). Therefore, both substitution (in Hairpin 1) and truncation (in ASO2) demonstrate the importance of the major-groove base triples. Dramatically, substituting A36 and A37 in the Hairpin 2 loop with two C residues disrupted the ASO2 binding by 130-fold. This confirms the importance of the minor-groove base triples and provides evidence that the minor-groove triples contribute more when they occur in the hairpin loop. The mutagenesis results for the two hairpin-ASO pairs suggest that the base triples observed in the pseudoknot structure are likely to be preserved in ASO-hairpin complexes.

### Hairpin Structures are Important for ASO Target Binding and Specificity

The 3D structure (FIG. 11A) suggests that the tertiary interactions with ASOs require the target RNA to be in a hairpin conformation. Studies tested if a stable hairpin stem is important for their interaction by truncating the Hairpin 1 stem from six base pairs to four (FIG. 12C). Strikingly, no binding could be detected between ASO1 and the truncated Hairpin 1. This result suggests that ASO1 recognizes the Hairpin 1 structure, even though it does not directly contact the deleted residues. Therefore, in a structure-based ASO design, this indirect readout further enhances target-binding specificity.

3D-ASO Design Templates that Bind to Hairpin-Flanking Regions

ASO1 and ASO2 bind to terminal loops of Hairpin 1 and 2, respectively (FIG. 13A, B). These are termed herein template A and B. Similar sets of tertiary interactions can be engineered into ASOs that hybridize to RNA strands flanking a target hairpin. By reconnecting the target RNA strands, Hairpin1Reconnect and Hairpin2Reconnect were derived that can similarly bind ASO1 and ASO2, respectively (FIG. 13C, D). Indeed, they do bind but with 3-6 fold decreased in affinity, which is attributed to changes in topological constraints. The availability of these derivative design templates, which are termed herein template C and D, broadens the applicability of our 3D design method. Four additional design templates are derived from the telomerase pseudoknot structure are shown in FIG. 13 (8 total templates).

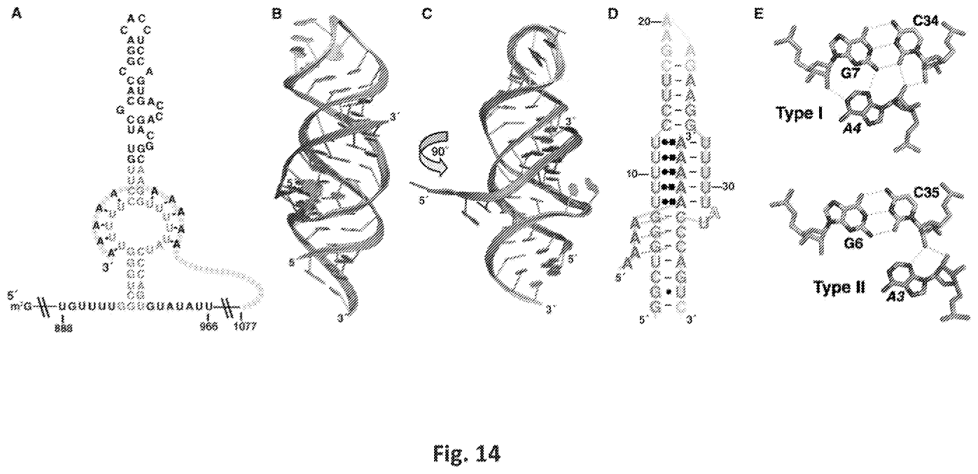

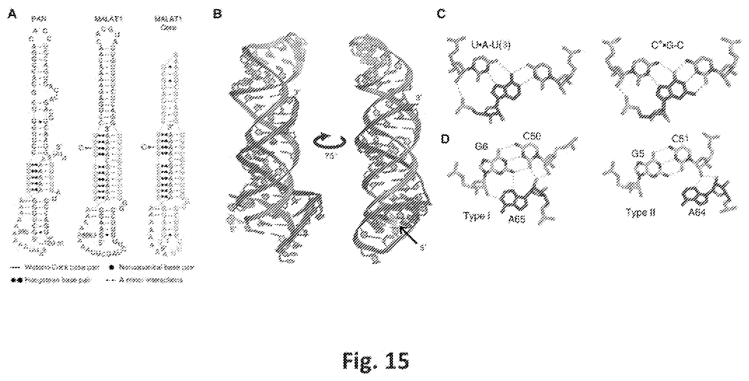

Template A-D require formation of a Hoogsteen pair (FIG. 13A-D, solid black lines), which impose substantial limitation to whether they can be applied to a target sequence. The instant disclosure provides four additional 3D-ASO design templates based on two RNA structures (Protein Data Bank accession codes 3P22 and 4PLX.) that resemble the overall architecture of the telomerase pseudoknot but use a dinucleotide bulge to connect the major- and minor-groove segments instead of the Hoogsteen pair (FIG. 13E-H). The polyadenylated nuclear (PAN) RNA is a noncoding RNA from Kaposi's sarcoma-associated herpesvirus. PAN contains an expression and nuclear retention element (ENE) that binds its poly(A) tail at the 3′ end, forms a pseudoknot together, and prevents its decay (FIG. 14). The second structure is a part of a human long noncoding RNA (lncRNA) called metastasis-associated lung adenocarcinoma transcript 1 (MALAT1). In the 3′ region of MALAT1, a stem loop binds a downstream A-rich tract and thereby forms a pseudoknot.

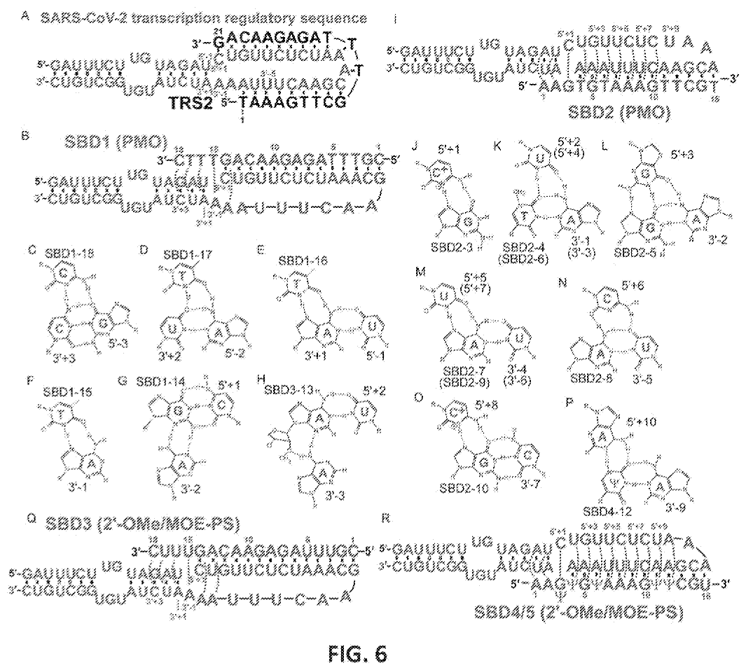

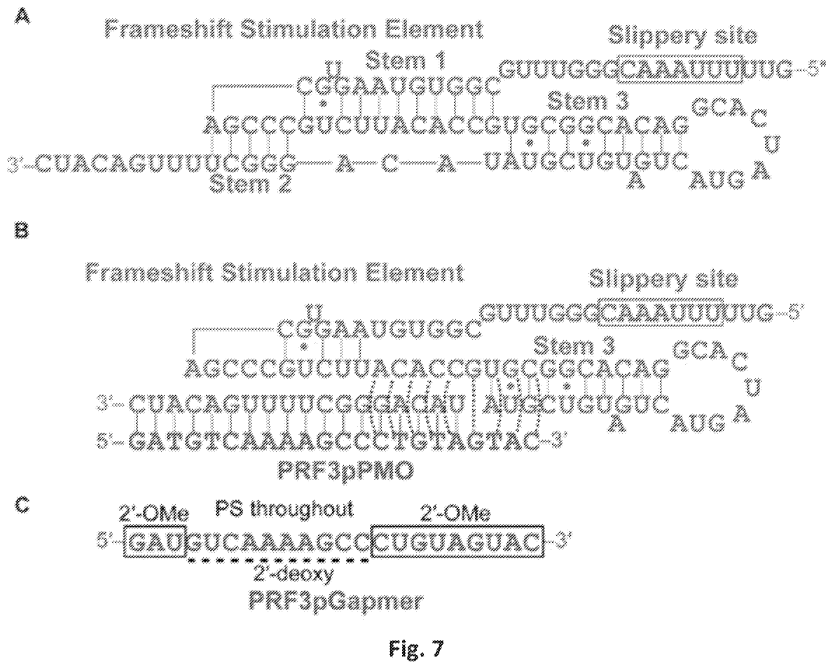

The PAN and MALAT1 structures contain at least five layers of major-groove base triples with geometry almost identical to those in the telomerase pseudoknot. Therefore, the major-groove segments of all eight design templates are the same. The minor-groove base triples of PAN and MALAT1 are also A-minor interactions, but with geometry different from those in the telomerase pseudoknot (FIGS. 6E and 7D).

The eight design templates in FIG. 13 target the most fundamental RNA structural element, a duplex. The duplex is often a part of an RNA hairpin as illustrated in FIG. 13, but template C, D, G, and H are applicable to any duplex. The duplex can be functionally important but may also (happen to) be close to a functionally important site. It can be evolutionarily conserved but may also be specific to one species. Therefore, the instant invention is widely applicable to ASO development.

Base Triples Compatible with the 3D-ASO Design Templates

To estimate what sequences are compatible with the structural templates, literature and the RNA Base Triple Database were surveyed^{14, 15} and major-groove and minor-groove base triples and Hoogsteen pairs were identified with geometries similar to those found in the structural templates. Using these compatible base triples in 3D-ASO design is an important part of this invention.

In the instant disclosure, a base triple is denoted as α⋅β-γ, in which residues β and γ form a WC base pair and residue a contacts the β-γ pair (primarily residue β or both β and γ) from either groove. 3D-ASO residues assume the positions of α or β depending on the template used and the location of these residues in the template.

### Major-Groove Base Triples

In the major-groove triple segments of template A, C, E, and G, 3D-ASO residues occupy the β position whereas the target RNA residues take the α and γ positions. For deciding these 3D-ASO residues, a table of major-groove base triples was generated as shown in FIG. 6, in which each row has the same a residues, each column has the same γ residues (both shown in green), and the β residues are indicated in red. These base triples cover all compatible α/γ combinations identified from the base triple database.

Several major-groove base triples involve non-WC pairs or modified bases. A-G pairs can form major-groove triple with either U or G (FIG. 16D, M). Similarly, G-A pairs can form a major-groove triple with C and G (FIG. 16H, L). G-U wobble pairs can form a major groove triple with A (FIG. 16M). Additionally, pseudouridine (ψ) enriches the Hoogsteen surface of U by introducing a hydrogen bond donor. ψ is an isomer of uridine in which the uracil base is attached to the ribose via a carbon-carbon bond instead of a nitrogen-carbon glycosidic bond. ψ is the most prevalent modified base found in natural RNAs, such as tRNAs. The nitrogen freed up from the glycosidic bond is on the Hoogsteen surface and can serve as a hydrogen-bond donor. Thereby, replace U with ψ in a U-A pair allows it to form major-groove triples with C and A (FIG. 16G, P). Introduction of non-canonical pairs and modified bases to ASOs to mediate tertiary interactions is an innovative aspect of this invention. These features are not necessarily used in every 3D-ASO design but when they are included, they provide important distinction of ASOs designed using the methods disclosed herein over the conventional designs.

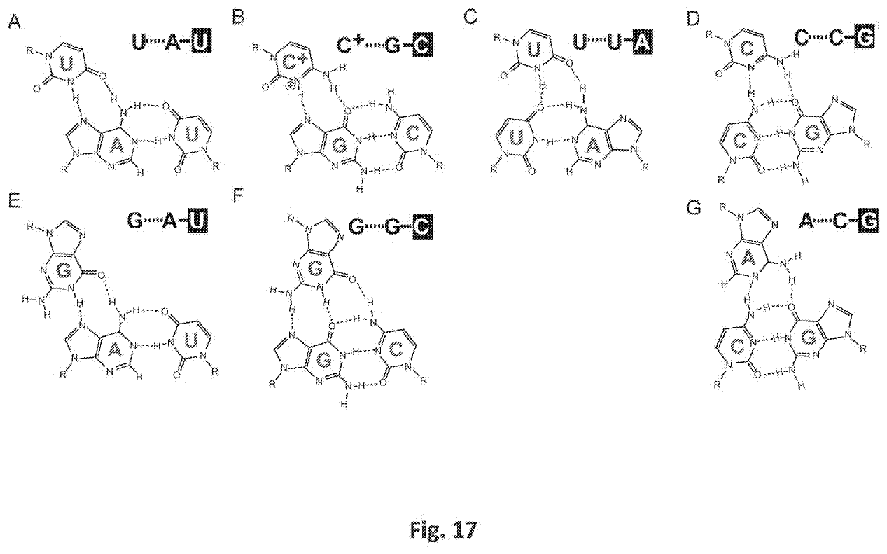

In the major-groove segments of template B, D, F, and H, 3D-ASO residues occupy the α position. For deciding these 3D-ASO residues, a table of major-groove base triples was generated as shown in FIG. 17, which contains a subset of the triples shown in FIG. 16. These triples are isosteric to the structural templates.

### Hoogsteen Pair

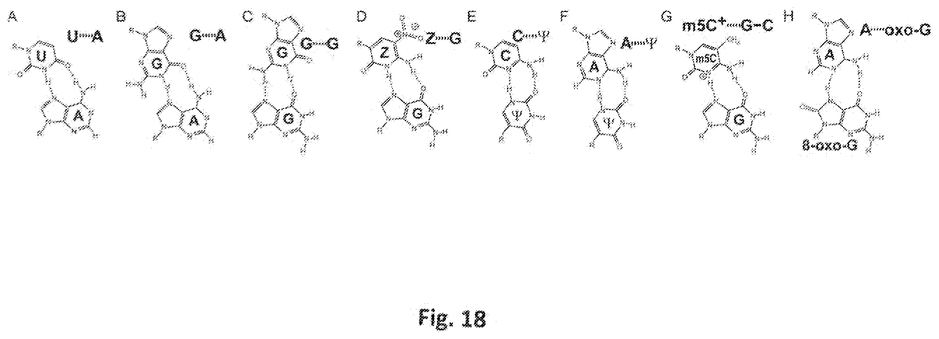

To satisfy the Hoogsteen pair in template A-D, the Nucleic Acids Database was surveyed to identify a group of base combinations that pair with similar geometries (FIG. 18). G can use its WC surface to pair with the Hoogsteen surface of A or G (FIG. 18B, C). Similarly, protonated C (C+) can pair with G (FIG. 18D). The requirement of protonation makes the C-G interaction pH-dependent and relatively weak at neutral pH (18). Additionally, a residue that has Hoogsteen surface capable of pairing with the WC surface of an A is desired. One solution is the use of pseudouridine (ψ), which can form Hoogsteen pair with both C and A (FIG. 17E, F).

### Minor-Groove Base Triples for Template A-D

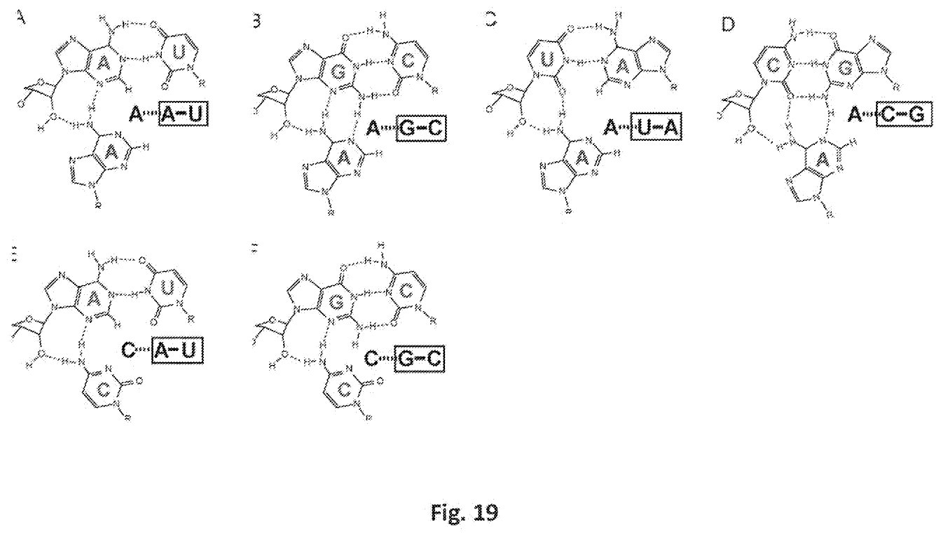

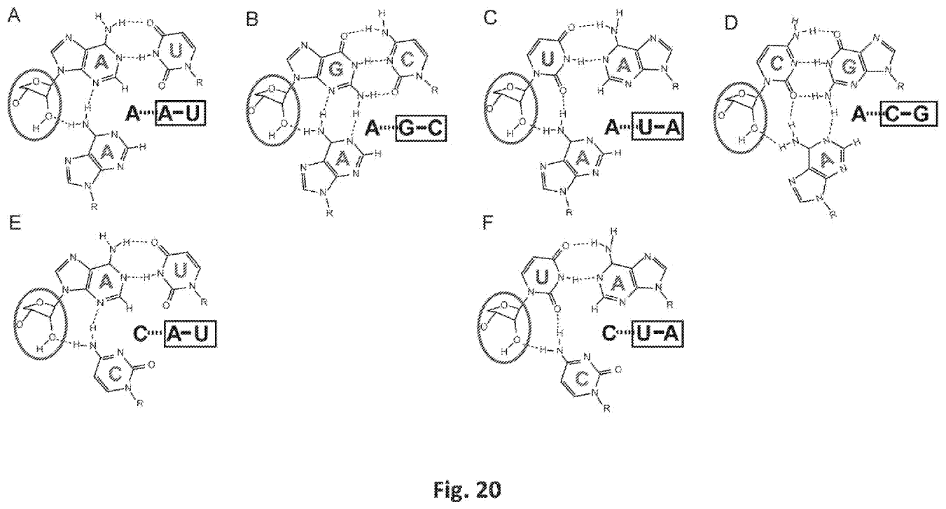

In template A and C, the 5′ region of ASO1 form minor-groove triples with the hairpin stem (FIG. 13A, C). At this position, (FIG. 19). In template B, 3D-ASO residues pair with 5′ side of the target hairpin loop and engage in minor-groove base triples with the other (FIG. 13B). In template D, 3D-ASO residues pairs with the 3′-neighboring region of a duplex and form minor-groove base triples with the 5′ neighboring strand (FIG. 13D). In these base triples, ASO residues assume the β position. Otherwise, essentially the same list of minor-groove base triples can be used (FIG. 20). ASO backbones are chemically modified. Commonly used modifications include phosphorothioate (PS), 2′-O-methyl (2′-OMe), 2′-O-methoxyethyl (2′-MOE), phospborodiamidate morpholino (PMO), locked nucleic acid (LNA), and peptide nucleic acid (PNA). How these modifications affect these minor-groove interactions remains to be determined experimentally

### Minor-Groove Base Triples for Template E-H

These base triples also belong to A-minor interactions but with geometries different from those in template A-D. Without being bound by a specific theory, it may be that only A residue can mediate such interactions.

### Illustrative Methods for 3D-ASO Designs

The procedure for designing 3D-ASOs typically includes the following steps:

### Identify the RNA Sequences to Target

These sequences include but not limited to exons, mRNA splice sites, intronic regions important for splicing, programmed ribosome frameshifting elements, microRNA-binding sites, protein-binding sites, polyadenylation signals, translation initiation sites, and other functionally important sites in 5′ and 3′ UTRs. This step is a common practice for both conventional and structure-based ASO design methods.

### Identify the Structures of the Target RNA

These structures can be either from previous reports or predicted using RNA secondary structure prediction programs such as RNAstructure¹⁶ and MFOLD¹⁷. Three-dimensional structures are welcome but not necessary. RNA secondary structure prediction programs most often provide a series of structures with low free energy levels. Usually the RNA structures occur with high frequency among the top predictions. These structures are considered with high confidence and thereby appropriate targets for 3D-ASO designs. The relatively weak or low-confidence structures are ignored in the designing process.

In addition the structure of a target RNA does not have to be the same in the free and ASO-bound states. Sometimes the goal is to disrupt a target RNA structure, as in the example presented below, 3D-ASOs targeting the SARS-CoV-2 programmed ribosome frameshifting element. In such situations, 3D-ASOs can harbor on a part of the structure or an alternative structure.

### Apply Design Templates

This disclosure describes general procedures and considerations. Once an appropriate hairpin or duplex structure has been identified, design templates can be applied to generate 3D-ASO sequences. A design template can give either 5′- or 3′-end sequences whereas the rest is determined by sequence complementarity. When two RNA structures are appropriately distanced in a target, both ends of an ASO may be designed using the 3D templates shown in FIG. 13.

For template A-D, the strongest constraint comes from the Hoogsteen pair bridging the major-groove and minor-groove segments. Therefore, the first step in designing 3D-ASOs based on these templates is to see if a Hoogsteen pair can be engineered. If yes, the design is extended on either side to optimize the major-groove and minor-groove interactions. If no, either explore dynamic variants of the target RNA structures (see below) or apply template E-H, which do not require a Hoogsteen pair to bridge the major- and minor-groove segments.

In templates A, C, E, and G, the 5′-end ASO residues recognize the targets' helical structures from their minor grooves but offer limited specificity to their sequences. Therefore, one can typically only dedicate 2-3 3D-ASO residues in the minor-groove triple segment. Information in FIG. 16 can be used to determine the 3D-ASO sequences in the major-groove segments.

In templates B, D, F, and H, the layer numbers of the minor-groove segments are largely dictated by the presence of A (or C) residues at specific positions in the targets. Information in FIG. 17 can be used to determine the 3D-ASO sequences in the major-groove segments.

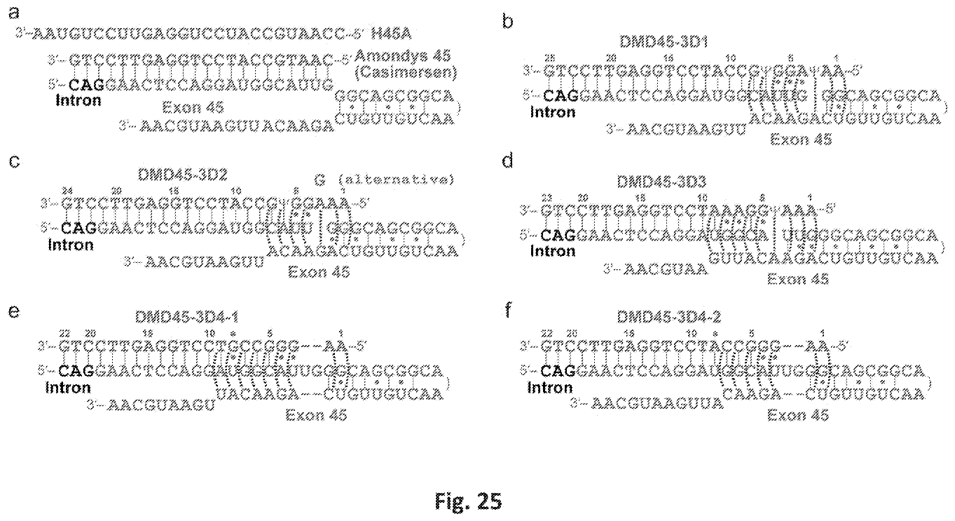

Dynamic variants of target RNA structures: RNA structures are known to be dynamic, with the base pairs at the ends of a hairpin or duplex opening and closing frequently. In some circumstances, additional base pairs (non-canonical and canonical) can be added. This property makes it possible to design 3D-ASO targeting these variants with low free-energy costs. Examples for targeting dynamic variants can be found in 3D-ASOs for treating Duchenne Muscular Dystrophy (FIG. 25B-D).

Considerations when targeting hairpin loops: RNA hairpin loops have been shown to mediate important biological functions such as binding proteins and regulating maturation of microRNAs. However, secondary structure prediction programs tend to introduce base pairs in the loop regions, which are not necessarily stable. In typical embodiments, one can usually disregard the base pairs that are separated from the hairpin stem. This opens some hairpin loops long enough so that 3D-ASOs with sufficient length (>13 nt) can be designed. The human genome contains 109 base pairs. It is estimated that at least 13 nt are needed to avoid unintended matches and thus non-specific binding.

### A Series of 3D-ASO Designs

The structure-based design method disclosed herein makes it possible to improve ASO drugs and drug candidates at various stages of development. Disclosure below provides a series of 3D-ASO designs as examples. As can be seen below, the 3D-ASOs are not fully complementary in sequence to their targets due to non-canonical pairs designed to optimize geometric compatibility and tertiary interactions. This is a signature feature of the design method disclosed herein. These designs also include modified bases such as pseudouridine (ψ).

### 3D-ASOs Targeting SARS-CoV-2 Viral RNA

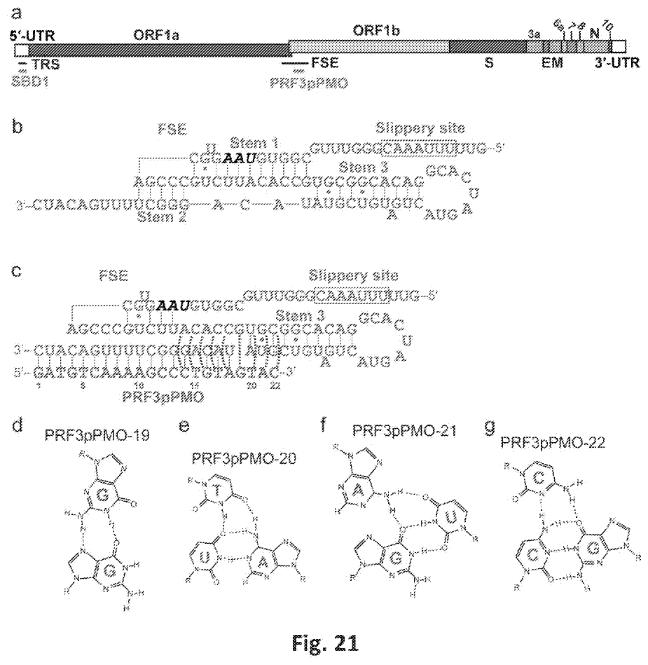

Several 3D-ASOs were designed that forge extensive base-triple and Hoogsteen base-pairing interactions with SARS-CoV-2 transcription regulatory sequence (TRS) and frameshift stimulation element (FSE) (FIG. 21a).

PRF3p that Targets the FSE

Coronaviruses require FSE to induce programmed −1 ribosomal frameshifting (PRF) and to translate the essential ORF1b. FSE contains a pseudoknot structure proceeded by a slippery sequence and an attenuator hairpin (FIG. 21b). During translation, a ribosome is stalled at the slippery site by the pseudoknot and this process gives the ribosome a chance to slip back by one nucleotide before being able to disrupt the pseudoknot and continue translating¹⁸. Without frameshifting, the ribosome encounters a downstream stop codon and fall off, producing protein 1a. With frameshifting, it completes the translation of ORF1b, generating protein 1a-1b.

A 3D-ASO was designed that disrupts the pseudoknot structure with the goal of eliminating the essential PRF for SARS-CoV-2. This pseudoknot contains stems 1-3 (FIG. 21b). This embodiment is designed to disrupt stems 1 and 2 so that the ribosome will not stall or will stall at a position downstream the slippery site. The design PRF3p, shown in FIG. 21c, follows template D. This 22-nt oligo hybridizes to a region immediately neighboring stem 3 on the 3′ side and engages in three major-groove, five minor-groove base triples and a Hoogsteen pair in-between (FIG. 21d-g). The phosphorodiamidate morpholino (PMO) modification was chosen because of its exceptional stability, neutral backbone, and safety as shown in approved drugs¹⁹.

SBD1 and SBD1-T15A that Target the TRS

Using template B, 3D-ASOs were designed that target the SARS-CoV-2 TRS. These 3D-ASOs improve a previously reported 1D design. In 2005, Neuman et al. designed nine ASOs targeting SARS-CoV RNA and found that several were effective to variable degrees in inhibiting the virus in cultured cells²⁰. The most effective one (named TRS2 in their paper but called PREV in this invention to avoid confusion with TRS, the abbreviation for transcription regulatory sequence) inhibits the virus at 10-20 μM by binding to the TRS. The authors proposed that the target TRS region folds into a hairpin. However, the 21-nt PREV is complementary to nearly the entire hairpin loop (FIG. 21a). As a consequence, either a substantial number of PREV bases cannot hybridize to the target, or they do so at the free-energy expense of having to disrupt the hairpin stem. In either case, PREV cannot achieve the intended binding affinity and specificity and any unpaired PREV residues only contribute to non-specific binding.

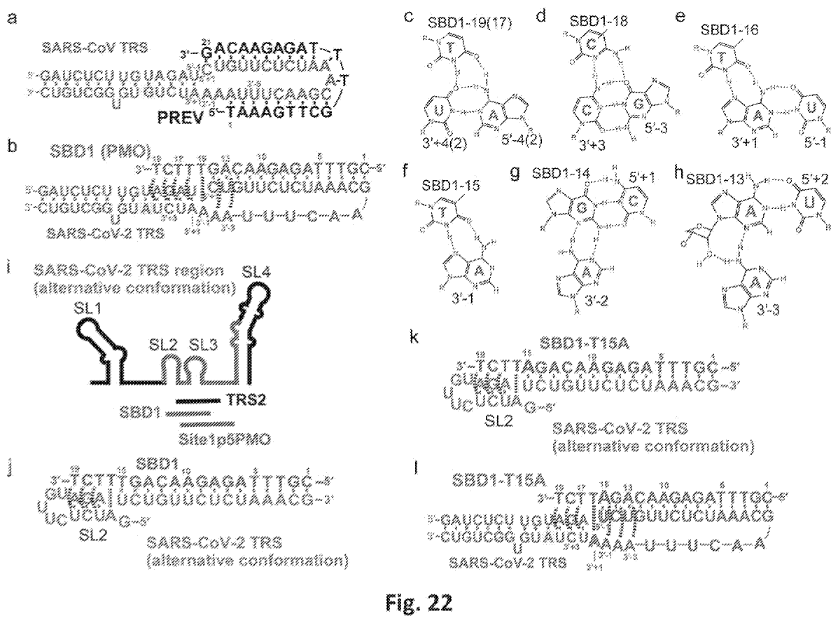

SBD1 was a 3D-ASO modified from PREV for targeting SARS-CoV-2 viral RNA. The sequences in the TRS region are almost identical between SARS-CoV and SARS-CoV-2, with a single nucleotide substitution that changes a U-G pair to a U-A pair (FIG. 22a). Therefore, ASOs targeting the TRS should similarly inhibit SARS-CoV-2. SBD1 shares 14 nucleotides with PREV, which form WC base pairs with 5′ region of the loop, leaving nine 3′-loop residues for connecting back to the hairpin stem (FIG. 22b). It also contains five residues (3′-TCTTT (SEQ ID NO: 25)) that form major groove base triples with the hairpin stem base pairs and a Hoogsteen pair with the 3′ terminal (3′-1) A residue of the loop (FIG. 22c-f). Two additional A residues at the 3′ loop region (3′-2 and 3′-3) can form a minor-groove base triple with the SBD1-loop duplex (FIG. 22g, h). These base-triple and Hoogsteen pairing interactions are accompanied by strong base stacking that further stabilizes the ASO-hairpin complex. Other SBD1 residues form WC pairs with the TRS hairpin loop, including three 5′ residues of the core TRS sequence ACGAAC (SEQ ID NO: 6). Thus, SBD1 is expected to disrupt the discontinuous transcription.

The design of 3D-ASOs for targeting the TRS region is complicated by alternative secondary structures. It is not uncommon for RNAs to fold into alternative structures; thus, structure-based ASO design methods should consider all of them. Secondary structure prediction and SHAPE analysis strongly suggested that 5′ region of SARS-CoV-2 genomic RNA folds into a structure that contains four stem loop structures (FIG. 22i)²¹⁻²⁶. The sequence proposed by Neuman et al. to fold into the TRS hairpin occupies SL2, SL3, 5′ region of SL4, and the linkers between them.

SBD1 can forge extensive tertiary interactions with SL2 of the alternative structure (FIG. 22j). SL2 contains a five-base-pair stem and a pentaloop. To bind SBD1 via template A, two base pairs at the base of SL2, G45-C59 and A46-U58, are replaced by interactions with SBD1, including a Hoogsteen pair between SL2-A246 and SBD1-T16, a non-canonical pair between SLC2-U58 and SBD1-T15, and WC pair between SL2-C59 and SBD1-G14. Importantly, three residues at the 3′-end of SBD1 (T19, C18, and T17) can form major-groove base triples with the SL2 stem and recognize the structure, resembling the expected interactions with TRS hairpin (compare FIG. 22j with 13b). No strong minor-groove interactions are expected. In this alternative binding mode, SL3 contains only four base pairs with a heptaloop, and is not expected to be very stable. SBD1 is designed to disrupt SL2 via WC base pairing. Overall, despite having to disrupt part of the target structures and engaging in fewer tertiary interactions with the alternative structure, SBD1 can remarkably bind both potential structures.

A variant of SBD1, TISA, was also designed. When binding to the alternative TRS structure, the T15A mutation replaces a T⋅U non-canonical pair with a A-U pair (FIG. 22k). When binding to the single-hairpin structure, the A15 residue cannot mediate the Hoogsteen pair with A at the 3′-1 position. Instead, A15 can pair with U at 5′−1 position and in turn disrupt its pairing with A(3′+1) (FIG. 22I). The T16 residue of SBD1-TISA can then form a Hoogsteen pair with A(3′+1) and allow T17-T19 to form major-groove base triples just like SBD1. Overall, the SBD1-TISA variant can forge tertiary interactions with both TRS structures, with the interaction with the alternative target structure more optimized.

### 3D-ASO Designs for Treating Duchene Muscular Dystrophy

Duchenne muscular dystrophy (DMD) is a rare genetic disorder that is characterized by progressive muscle weakening and wasting, almost exclusively affecting boys. According to the NIH Genetic and Rare Diseases (GARD) Information Center, DMD affects approximately 1 in 3,500 to 5,000 male births worldwide. There is no cure. Physical therapy and hormonal medications are used to control symptoms and improve quality of life. Even with improved care in recent years, patients become wheelchair-bound in their early teens and have much shorter life span, typically dying in their 30s.

DMD is caused by mutations in the gene encoding a protein called Dystrophin, which supports muscle fiber strength. In the past three years, ASO drugs showed promise in treating DMD. Four ASO drugs have been approved by the US FDA for treating DMD.

### Eteplirsen and Golodirsen

Eteplirsen and golodirsen were developed by Sarepta Therapeutics to treat specific types of DMD that are caused by mutations that change the reading frame of Dystrophin translation. About 13% and 8% of DMD patients fall in these categories. Eteplirsen is a 30-nucleotide ASO that binds to exon 51 and causes it to be skipped during pre-mRNA splicing and thereby restore proper translation reading frame. This “exon skipping therapy” produces a Dystrophin protein that is partially functional and reduces the severity of the disease. Golodirsen is a 25-nucleotide ASO that binds and causes exon 53 to be skipped.

The accelerated approval of Eteplirsen in 2016 was both exciting and controversial. The drug was able to partially restore Dystrophin protein production. As the first in its class, it proved that the novel exon skipping mechanism works. However, eteplirsen is only marginally effective clinically and does not show significant improvement in the standard walking test. Two FDA review panel members resigned in protest. In 2018, the European Medicines Agency refused to approve it. Furthermore, eteplirsen is expensive, costing $300,000 annually per person. Therefore, it is important to refine the design with higher efficacy and reduce costs.

### 3D-ASO Designs for Skipping Exon 51

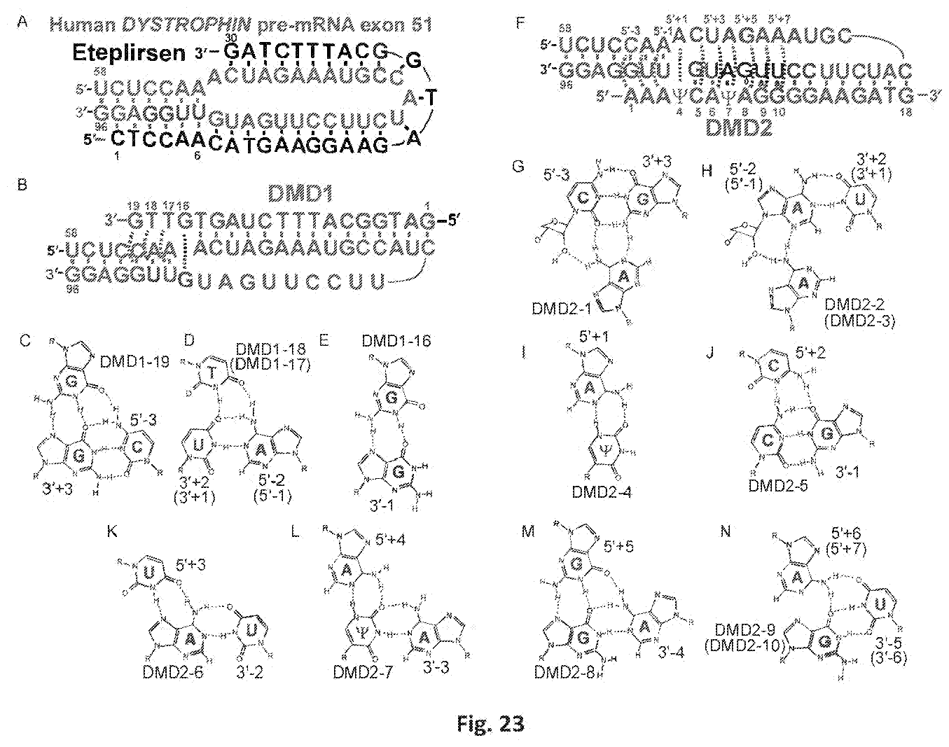

The eteplirsen-binding site and surrounding sequences on exon-51 fold to a hairpin structure that is predicted with high confidence (FIG. 23A). The base pairing between eteplirsen and its target is structurally incompatible with the hairpin conformation, i.e., eteplirsen has to break the base pairs in the hairpin before it can bind fully with exon. The energy it spends to do so will reduce its binding affinity and specificity.

Two 3D-ASOs (DMD1 and DMD2) were designed that are not only compatible with the hairpin structure, but also forge extensive additional tertiary interactions (FIG. 23B-N). DMD1 and DMD2 bind to 5′ and 3′ regions of the hairpin loop (FIG. 23B, H), respectively. They both form many hydrogen bonds with the target RNA (FIG. 23C-E, 23G-N), more than those found in WC base pairs. The free energies gained from these additional interactions and saved from not having to disrupt the hairpin stem should allow our ASOs to bind to the target RNA with high affinity and specificity even though they are shorter (19 and 18 nucleotides for DMD1 and DMD2, respectively). The shorter sequences come with great benefits including easier delivery, lower costs of synthesis, and lower chance of triggering innate immunity.

### 3D-ASO Designs for Skipping Exon 44

The human DMD exon 44 sequence (148 nt) is
GCGATTTGACAGATCTGTTGAGAAATGGCGGCGTTTTCATTATGATATAAAG ATATTTAATCAGTGGCTAACAGAAGCTGAACAGTTTCTCAGAAAGACACAAA TTCCTGAGAATTGGGAACATGCTAAATACAAATGGTATCTTAAG (SEQ ID NO: 1)

The 24-nt H44A ASO that was reported by Wilton et al. in 2007²⁷ and subsequently studied by Carrie Miceli's group²⁸ has the following sequence: 5′-UGUUCAGCUUCUGUUAGCCACUGA-3′ (SEQ ID NO: 2)

H44A anneals to the exon residues 61-84.

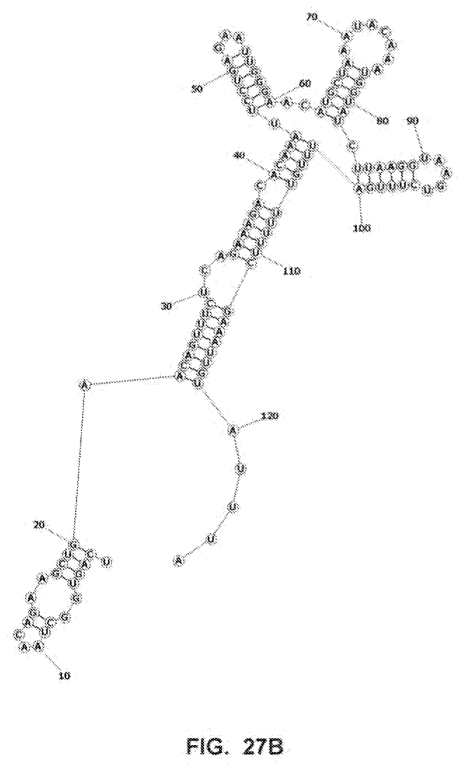

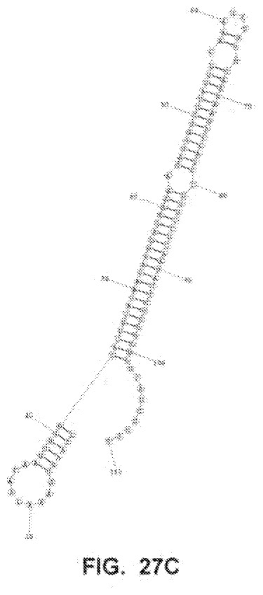

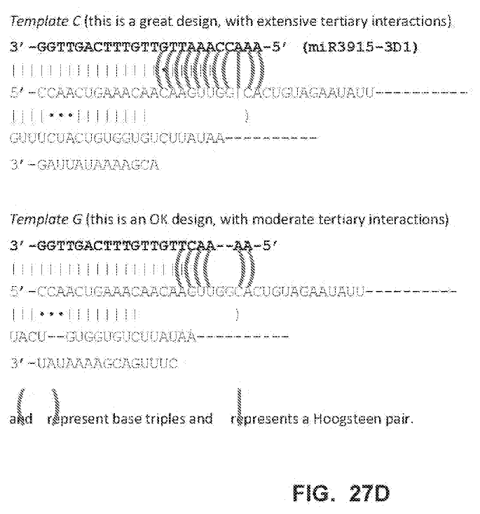

Secondary structure prediction of exon 44 gives the structure (residues 48-116) shown in FIG. 27A:

Note that 47 needs to be added to the numbering in the drawing to get the position in exon 44.

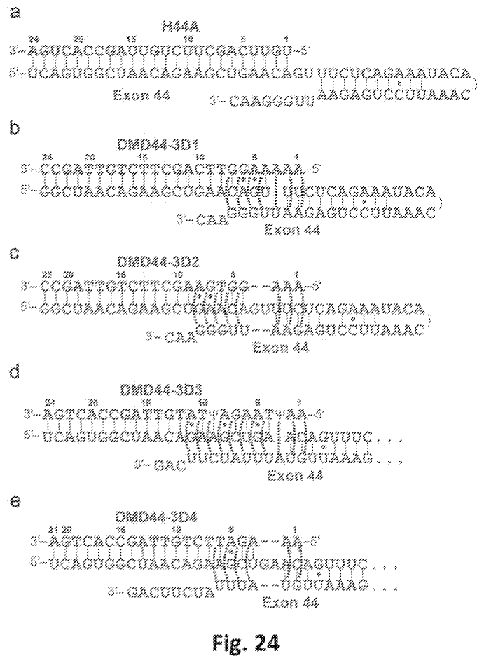

H44A anneals to positions 14-37, the nearest residue of which is two nucleotides away from the third stem loop (see also FIG. 24a).

DMD44-3D1 and DMD44-3D2 were designed based on template C and G, respectively (FIG. 24b, c).

Additionally, the methods disclosed herein can consider the folding of ASO-binding sites in the context of pre-mRNAs. This is routinely done in typical methods of the invention. The presence of 3′ intron gave rise to an alternative structure as shown in FIG. 27B:

Note that 60 need to be added to the numbering to get the position in exon 44, which ends at residue 88 on the graph. Residues 89 and on belong to intron 44-45.

DMD44-3D3 and DMD44-3D4 were designed based on template C and G, respectively (FIG. 24d, e).

### 3D-ASO Design for Skipping DMD Exon 45

The human exon 45 sequence (176 nt) is
GAACUCCAGGAUGGCAUUGGGCAGCGGCAAACUGUUGUCAGAACAUUGA AUGCAACUGGGGAAGAAAUAAUUCAGCAAUCCUCAAAAACAGAUGCCAGU AUUCUACAGGAAAAAUUGGGAAGCCUGAAUCUGCGGUGGCAGGAGGUCUG CAAACAGCUGUCAGACAGAAAAAAGAG (SEQ ID NO: 3)

The immediate 5′ intron 50-nt sequence is
UAAAAAGACAUGGGGCUUCAUUUUUGUUUUGCCUUUUUGGUAUCUUACAG (SEQ ID NO: 4)

The 26-nt H45A ASO that was reported by Wilton et al. in 2007²⁷ and subsequently studied by the Miceli group²⁸ anneals to the 5′-splice site, with the underlined resides pairing with the neighboring intron (FIG. 25a). Comparing to H45A, the FDA-approved Amondys 45 (Casimersen) removed a C on the 5′-end and UAA (SEQ ID NO: 5) on the 3′-end. It has only three residues annealing to the 5′-intron. Such a short pairing with the intron is sufficient to cause exon 45 skipping.

Secondary structure prediction of exon 45 with 50 nt on each flanking region revealed a hairpin that is consistently present, in 19 out of the top 20 predicted structures. This hairpin sequence is underlined in the exon 45 sequence shown above. In the context of this structure, removal of the C residue at the 5′-end of H45A avoids the competition for pairing with the G at the 5′-end of the hairpin and allows the Amondys 45-target RNA duplex to stack with the hairpin. Therefore, Amondys 45 is a 2D design.

Several 3D-ASOs were designed to improve Amondys 45 (FIG. 25b-f). DMD45-3D1 is a design by applying template C to the hairpin as predicted (FIG. 25b). In DMD45-3D1, a ψ at the third position forms a Hoogsteen pair with a 3′ A neighboring the hairpin. Two A residues at the 5′ end form minor-groove base triples with the hairpin stem. Five layers of major-groove base triples form between residues 4-8 of DMD45-3D1 and the target RNA. The net change from Amondys 45 is to replace the CAAT (SEQ ID NO: 8) at the 5′ end with AAψAGGψ (SEQ ID NO: 7).

DMD45-3D2 and DMD45-3D3 explore the malleability of RNA helices. DMD45-3D2 assumes that the hairpin stem can be extended by a G-A non-canonical pair (FIG. 25c). As such, the Hoogsteen pair is mediated by an A at the third position of DMD45-3D2 and a G residue 3′ of the target hairpin. Compared to DMD45-3D1, DMD45-3D2 replaces the AAA (SEQ ID NO: 9) at the 5′ end with AAA (SEQ ID NO: 10). DMD45-3D3 assumes that the hairpin stem can be further extended by two additional base pairs, U⋅G and U-A (FIG. 25d). Compared to DMD45-3D1, DMD45-3D3 replaces the AAψA (SEQ ID NO: 9) at the 5′ end with AAAψ (SEQ ID NO: 11).

DMD45-3D4-1 and -2 are designs by applying template G to the hairpin (FIG. 25e, f). In both 3D-ASOs, two 5′ A residues form minor-groove base triples with the hairpin stem. Two 5′ residues (UG) neighboring the hairpin bulge out. The two 3D-ASOs defer from each other by the number of major-groove base triples engineered. DMD45-3D4-1 forms seven layers of major-groove base triples with the target RNA, with three of them involving non-canonical base pairs (FIG. 25e). DMD45-3D4-2 is a less ambitious design, with five layers of major-groove base triples involving two non-canonical base pairs (FIG. 25f). Biochemical and functional assays can be used to compare these designs and determine which one is most potent.

### 3D-ASOs for Regulating Human FAN1

FAN1 is Fanconi-associated nuclease 1. It is also called KIAA1018, MTMR15, FANCD2/FANC1-associated nuclease 1. It is required for DNA interstrand crosslink repair, but is not involved in DNA double-strand break resection. FAN1 is recruited to DNA damage sites by monoubiquitinated FANCD2. FAN1 has a UBZ-type ubiquitin-binding domain at the N-terminus, a SAP-type DNA binding domain in the middle, and a nuclease domain termed the “VRR_nuc” domain at the C-terminus. It has endonuclease activity toward 5′ flaps and 5′-3′ exonuclease activities, which require the VRR_nuc domain.

The FAN1 protein is expressed in two major isoforms, as indicated in the UniProt database. Isoform 1 is the full length containing 1017 a.a. and all the functional domains. Isoform 2 contains 533 a.a., with residues 1-526 identical to isoform 1 and residues 527-533 changed from AKALAGQ (SEQ ID NO: 12) in isoform 1 to FCWLLLQ (SEQ ID NO: 13). Isoform 2 misses residues 534-1017 in the full-length protein, including the VRR-nuc domain, and thus is not expected to be functional. Therefore, it should be possible to increase the functional FAN1 expression level by diverting the mRNAs for isoform 2 to the isoform 1 during RNA processing. Seven additional isoforms are computationally predicted, containing 430, 237, 573, 318, 587, 103, and 298 a.a. in length.

It was observed that the mRNA isoforms coding for the shorter protein isoform are generated by alternative polyadenylation, not by alternative splicing. A search of the human protein atlas found the following five mRNA variants:
1. FAN1-201 (ENST00000362065.8) encodes 1017 a.a., containing 15 exons (13 coding) and 4,891 nt in length. The lengths (nt) of exons and introns are noted below, with the numbers in parenthesis indicating the lengths of introns.
   139(500)1,386(2,220)141(2,355)202(3,042)234 . . . (4306)1,543
   The last exon (1,543 nt) contains multiple copies of polyadenylation signal (PAS) sequences.
2. FAN1-202 (ENST00000561594.5) encodes 533 a.a. (isoform 2), containing 4 exons (3 coding) and 2,738 nt in length.
   36(626)1,386(2,220)141(2,355)1,175
   Exon 4 has 1,175 nt, which includes the 202 nt of exon 4 and 973 nt of intron 4-5 in FAN1-201. The coding extends 22 nt before hitting the TAA (SEQ ID NO: 14) stop codon. It does not include 3′ region of intron 4-5 or exon 5 of FAN1-201. Importantly, residues 1149-1154 of exon 4 contains a PAS (AUUAAA (SEQ ID NO: 15), PAS1) that is about 20 nt from the 3′ end (the cleavage site), indicating that FAN1-202 is generated via alternative polyadenylation.
3. FAN1-203 (ENST00000561607.5) encodes the 533-residue isoform 2, containing 4 exons (3 coding) and 2,690 nt in length.
   133(487)1,386(2,220)141(2,355)1,030
   Exon 4 contains 1,030 nt, which is identical to exon 4 of FAN1-202 except being shorter. Importantly, resides 995-1000 of exon 4 contains a PAS (AAUAAA, (SEQ ID NO: 16) PAS2) that is about 30 nt from the 3′ end, indicating that FAN1-203 is generated by alternative polyadenylation at this site. Note that this PAS is also present in FAN1-202.
4. FAN1-205 (ENST00000562892) encodes a 105-a.a. protein, containing 3 exons (all coding) and 344 nt in length.
   81(3,527)141(2,355)122
   Exon 3 contains 122 nt that is identical to exon 4 of FAN1-201, -202, and -203 variants except being shorter. This variant does not have a stop codon (terminating in the coding region) and appears to be incomplete in the 3′ end.
5. FAN1-207 (ENST00000565466) encodes the 533-a.a. protein, containing 4 exons (3 coding) and 2,304 nt in length.
   119(500)1,386(2,220)141(2,355)658
   Exon 4 contains 658 nt that is identical to exon 4 of FAN1-202 and -203 except being shorter. It is not clear what PAS sequence directs the alternative adenylation at this site.
      In sum, production of transcripts FAN1-202 and FAN1-203 are clearly directed by two alternative PAS sequences, providing an opportunity for drug discovery.

A recent paper reports that ASOs can be used to inhibit polyadenylation of shorter transcript and increase the production of longer RNAs 29. Four ID-ASO sequences were designed that potentially modulate polyadenylation of FAN1 and effectively the full-length protein expression.
3′-AAAUUACAAAUGAACGAAUAUUUUA-5′ (SEQ ID NO: 17) (target PAS1 exon 4 res. 1154-1129)
5′-UUUAAUGUUUACUUGCUUAUAAAAU-3′ (SEQ ID NO: 18) (FAN1ASO1, 25 nt)
3′-GUCGGUCUCCCACCGAUUUAAAUUA-S′ (SEQ ID NO: 19) (target PAS1 exon 4 res. 1173-1149)
5′-CAGCCAGAGGGUGGCUAAAUUUAAU-3′ (SEQ ID NO: 20) (FAN1ASO2, 25 nt)
3′-AAAUAACUUCAUGAAAAAUUUUUAG-5′ (SEQ ID NO: 21) (target PAS2 exon 4 res. 1000-976)
5′-UUUAUUGAAGUACUUUUUAAAAAUC-3′ (SEQ ID NO: 22) (FAN1ASO3, 25 nt)
3′-UAAUUAAUGUUCAUACAUUAAAUAA-5′ (SEQ ID NO: 23) (target PAS2 exon 4 res. 1021-995)
5′-AUUAAUUACAAGUAUGUAAUUUAUU-3′ (SEQ ID NO: 24) (FAN1ASO4, 25 nt)

To perform 3D-ASO design, the embodiment of the methods disclosed herein included performing a secondary structure prediction for the exon 4 of both FAN1-202 and FAN-203 mRNAs using RNAstructure and found both PAS sequences are present as part of high-confidence hairpin structures. Residues 1105-1155 for a hairpin that is present in all top 20 predictions. The PAS sequence (AUUAAA (SEQ ID NO: 15)) is present at the 3′ end (residue 1149-1154).

Residues 993-1022 form hairpin that appears in the top 19 predictions for FAN1-203 exon 4, as well as in 19 out of the top 20 predictions for the longer FAN1-202 exon 4. The PAS (AAUAA (SEQ ID NO: 26)) is located at the 5′ end (residues 995-1000).

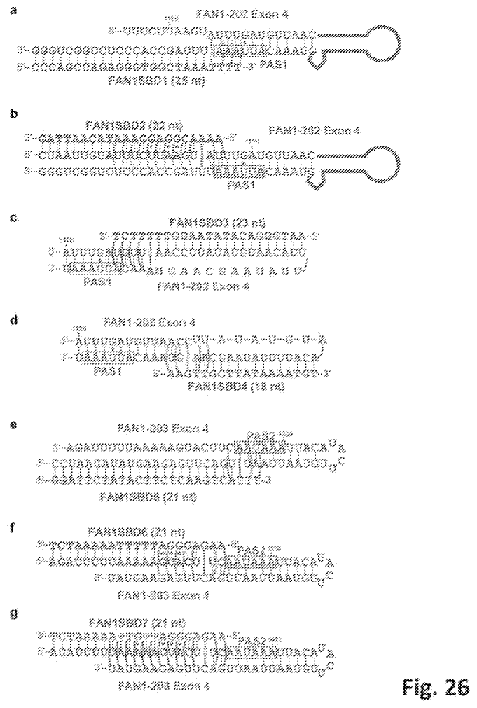

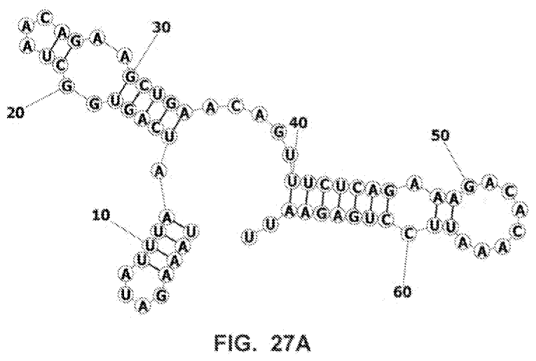

These structures can potentially sequester the PAS′ and prevent access by the mRNA cleavage and polyadenylation machinery. This observation may explain why these PAS sites are only used a fraction of time. Several ASO sequences that target these structures and their flanking regions have been designed(FIG. 26).

### Indications

Two lead 3D-ASO sequences (SBD1 and PRF3pPMO) strongly inhibit SARS-CoV-2 viral replication in culture HEK293 cells. The inhibitory activity of PRF3pPMO and its variants toward the frameshifting stimulatory element is further confirmed using a dual luciferase report assay. MTT assays showed that these 3D-ASOs are not cytotoxic (SBD1) or only toxic at very high concentration (PRF3pPMO).

### REFERENCES FROM TEXT ABOVE

1. Bennett, C. F. Therapeutic antisense oligonucleotides are coming of age. Annu. Rev. Med. 70, 307-321 (2019).
2. Stein, C. A. & Castanotto, D. FDA-Approved Oligonucleotide Therapies in 2017. Mol. Ther. 25, 1069-1075 (2017).
3. Crooke, S. T., Witztum, J. L., Bennett, C. F. & Baker, B. F. RNA-Targeted Therapeutics. Cell Metab. 27, 714-739 (2018).
4. Zamecnik, P. C. & Stephenson, M. L. Inhibition of Rous sarcoma virus replication and cell transformation by a specific oligodeoxynucleotide. Proceedings of the National Academy of Sciences of the United States of America 75, 280-284 (1978).
5. Stephenson, M. L. & Zamecnik, P. C. Inhibition of Rous sarcoma viral RNA translation by a specific oligodeoxyribonucleotide. Proceedings of the National Academy of Sciences of the United States of America 75, 285-288 (1978).
6. Kuijper, E. C., Bergsma, A. J., Pim Pijnappel, W. W. M. & Aartsma-Rus, A. Opportunities and challenges for antisense oligonucleotide therapies. J. Inherit. Metab. Dis. (2020).
7. Bennett, C. F., Krainer, A. R. & Cleveland, D. W. Antisense oligonucleotide therapies for neurodegenerative diseases. Annu. Rev. Neurosci. 42, 385-406 (2019)
8. Quemener, A. M. et al. The powerful world of antisense oligonucleotides: From bench to bedside. Wiley Interdiscip. Rev. RNA, e1594 (2020).
9. Lim, K. R. Q. & Yokota, T. Invention and Early History of Gapmers. Methods Mol. Biol. 2176, 3-19 (2020).
10. Dominguez, D. et al. Sequence, Structure, and Context Preferences of Human RNA Binding Proteins. Mol. Cell 70, 854-867 e859 (2018).
11. Yang, D. & Leibowitz, J. L. The structure and functions of coronavirus genomic 3′ and 5′ ends. Virus Res. 206, 120-133 (2015).
12. Cate, J. H. et al. RNA tertiary structure mediation by adenosine platforms. Science (New York, N.Y 273, 1696-1699 (1996).
13. Nissen, P., Ippolito, J. A., Ban, N., Moore, P. B. & Steitz, T. A. RNA tertiary interactions in the large ribosomal subunit: the A-minor motif. Proc. Natl. Acad. Sci. U.S.A. 98, 4899-4903 (2001).
14. Abu Almakarem, A. S., Petrov, A. I., Stombaugh, J., Zirbel, C. L. & Leontis, N. B. Comprehensive survey and geometric classification of base triples in RNA structures. Nucleic Acids Res. 40, 1407-1423 (2012).
15. Coimbatore Narayanan, B. et al. The Nucleic Acid Database: new features and capabilities. Nucleic Acids Res. 42, D114-122 (2014).
16. Lorenz, R. et al. ViennaRNA Package 2.0. Algorithms Mol. Biol. 6, 26 (2011).
17. Zuker, M. Mfold web server for nucleic acid folding and hybridization prediction. Nucleic acids research 31, 3406-3415 (2003).
18. Namy, O., Moran, S. J., Stuart, D. I., Gilbert, R. J. & Brierley, I. A mechanical explanation of RNA pseudoknot function in programmed ribosomal frameshifting. Nature 441, 244-247 (2006).
19. Nan, Y. & Zhang, Y. J. Antisense phosphorodiamidate morpholino oligomers as novel antiviral compounds. Front. Microbiol. 9, 750 (2018).
20. Neuman, B. W. et al. Inhibition, escape, and attenuated growth of severe acute respiratory syndrome coronavirus treated with antisense morpholino oligomers. J. Virol. 79, 9665-9676 (2005).
21. Iserman, C. et al. Genomic RNA Elements Drive Phase Separation of the SARS-CoV-2 Nucleocapsid. Mol. Cell 80, 1078-1091 e1076 (2020).
22. Manfredonia, I. et al. Genome-wide mapping of SARS-CoV-2 RNA structures identifies therapeutically-relevant elements. Nucleic Acids Res. 48, 12436-12452 (2020).
23. Rangan, R. et al. RNA genome conservation and secondary structure in SARS-CoV-2 and SARS-related viruses: a first look. RNA 26, 937-959 (2020).
24. Simmonds, P. Pervasive RNA Secondary Structure in the Genomes of SARS-CoV-2 and Other Coronaviruses. MBio 11 (2020).
25. Tavares, R. C. A., Mahadeshwar, G., Wan, H., Huston, N. C. & Pyle, A. M. The global and local distribution of RNA structure throughout the SARS-CoV-2 genome. J. Virol. (2020).
26. Sun, L. et al. In vivo structural characterization of the SARS-CoV-2 RNA genome identifies bost proteins vulnerable to repurposed drugs. Cell 184, 1865-1883 e1820 (2021).
27. Wilton, S. D. et al. Antisense oligonucleotide-induced exon skipping across the human dystrophin gene transcript. Mol. Ther. 15, 1288-1296 (2007).
28. Barthelemy, F. et al. Targeting RyR Activity Boosts Antisense Exon 44 and 45 Skipping in Human DMD Skeletal or Cardiac Muscle Culture Models. Mol. Ther. Nucleic Acids 18, 580-589 (2019).
29. Naveed, A. et al. NEAT1 polyA-modulating antisense oligonucleotides reveal opposing functions for both long non-coding RNA isoforms in neuroblastoma. Cell Mol. Life Sci. 78, 2213-2230 (2021).

As discussed in detail below, the instant disclosure provides a structured-based method for designing antisense oligonucleotides (ASOs) for therapeutics and for biomedical research. A core concept of these methods is to incorporate base triples and non-canonical base pairs in ASO designs to achieve high binding affinity and specificity. The instant disclosure provides the guiding principles of this methodology and illustrative designs of ASO sequences for a range of high-value targets.

At the heart of the methods disclosed herein is a set of guiding principles for structure-based ASO design. The design principles were inspired by existing RNA structures. This instant disclosure further validates these design principles using, for example, sequences derived from human telomerase RNA as model systems. Truncation and mutation analyses clearly indicate that the tertiary interactions are critical for the ASO-hairpin association. The disclosure illustrates the design of ASOs that target SARS-CoV-2 RNA and a manuscript that describes the design principles and validation is found in Li et al., Structure-based design of antisense oligonucleotides that inhibit SARS-CoV-2 replication. bioRxiv [Preprint]. 2021 Aug. 24:2021.08.23.457434. doi: 10.1101/2021.08.23.457434. PMID: 34462746; PMCID: PMC8404888; which is incorporated herein by reference.

Because RNAs are involved in nearly every aspect of biology and diseases, the methods disclosed herein a widely applicable to specifically regulate almost any target RNAs.

### Example 1: Title: Structure-Based Design of Antisense Oligonucleotide Drugs

The instant disclosure provides structure-based design methods for antisense oligonucleotides (ASOs) and uses these discoveries to develop ASO drugs for treating a wide range of diseases, including (COVID-19). ASOs are a new class of drugs that are especially useful for fighting emerging infectious diseases. They are single-stranded synthetic oligonucleotides that specifically bind target RNAs and elicit desired biological and therapeutic effects. Recently, several ASO drugs have been approved by the U.S. Food and Drug Administration, marking the coming of age of the technology after four decades of development. In principle, ASOs can be rapidly deployed to target any pathogenic sequences and thereby treat a wide range of diseases including Duchenne muscular dystrophy and coronavirus disease 2019 (COVID-19). Additionally, the sequences of ASOs can be easily modified to counter drug-resistant viral strains.

Conventional ASO designs strictly follow the rule of Watson-Crick (WC) base pairing along linear target sequences. Usually 18-25 nucleotides are required to achieve sufficient affinity and specificity. However, viral RNAs often fold into secondary and tertiary structures that can interfere with ASO hybridization. Therefore, conventional ASO design strategies either avoid targeting structured regions or simply ignore these structures and rely on screening large numbers of ASOs to identify high-affinity binders. The designs disclosed herein are compatible with target structures in three-dimensional (3D) space and engage in tertiary interactions in addition to WC base pairing. ASOs designed this way bind targets with enhanced affinity due to the tertiary interactions gained and the free energy saved for not having to disrupt target structures. Moreover, binding specificity is elevated beyond what can be achieved just by WC pairing interactions since the ASOs do not only recognize the target sequences but also the shapes and hydrogen-bonding patterns. The instants disclosure provides a set of principles on how to design ASOs based on structures and identified two design templates. The binding study confirmed the importance of tertiary interactions in these designs.

As a new type of drug, ASOs are revolutionizing medicine (1, 2). ASO hybridizations do not rely on complementarity to protein-binding surfaces and in principle any sequences could be targeted following the Watson-Crick (WC) base pairing rule. Hence ASO therapeutics can regulate cellular pathways that are not readily druggable with small molecules (3). In 1978, Zamecnik and Stephenson demonstrated that ASOs can inhibit viral replication in vitro (4, 5). In 1998, the first ASO drug Fomivirsen received approval, for treating cytomegalovirus retinitis (CMV) in immunocompromised patients. Although its market authorization was later withdrawn because the number of CMV infection cases was dramatically reduced by the development of HIV drugs, Fomivirsen demonstrated the feasibility and potential of ASOs as antivirals. Since 2013, six ASO drugs gained approval to treat a variety of genetic disorders (Fomivirsen, Mipovirsen, Eteplirsen, Nusinersen, Inotersen, Volansorsen and Golodirsen). To date, 66 ASO drug candidates are in clinical trials for a wide range of diseases (6, 7), including three for hepatitis B (Pharmaprojects database).

ASO therapeutics hybridize to target RNAs and elicit desired therapeutic effects via several mechanisms, such as inducing RNA degradation by cellular RNase H, inhibiting translation, causing exon inclusion or skipping during pre-mRNA splicing, competing with RNA-binding proteins and microRNAs for regulation, and inhibition of viral RNA replication, transcription, and viral particle assembly. To achieve serum stability and favorable pharmacokinetics, ASO drugs are chemically modified to be resistant to the nucleases that are abundant in serum and cells. These chemical modifications must be compatible with the mechanisms of action.

A class of ASOs is DNA oligos that form DNA/RNA duplexes with targets and causes their degradation by cellular RNase H. The earliest ASOs such as Fomivirsen contained only a phosphorothioate (PS) modification in the backbone, which increases nuclease resistance but does not interfere with RNase H activity. The second-generation RNase H-dependent ASOs contain additional 2′-O-(2-methyloxyethyl) (2′-MOE) modifications in the 10 outermost residues of a 20mer oligo. These residues contribute to binding target RNAs but are not compatible with RNase H cleavage. By narrowing the window of RNase H cleavage to the central 10-nucleotide region, these “gapmers” have improved specificity. Mipomersen, Inotersen, and Volanesorsen are gapmers. Another category of ASOs (such as Eteplirsen, inotersen, and golodirsen) does not depend in RNase H but instead sterically blocks the targets from interacting with cellular or viral machinery. These “steric blockers” contain 2′-MOE, phosphorodiamidate morpholino (PMO), or other backbone modifications, which enhance both target-binding affinity and nuclease resistance (2, 7). These mechanisms and chemical modifications are contemplated in the structure-based ASO designs.

Single-stranded RNAs tend to form intramolecular base pairs, which stack with each other to form double-stranded helices and higher order structures. These structures are often important for biological functions such as recruiting RNA-binding proteins (8). Viruses often contain RNA hairpins in their 5′- and 3′-UTRs important for viral replication and translation and in the ribosome frame shifting (RFS) sites responsible for continuing translation of downstream viral proteins. These structures often hinder ASO hybridization, as their pairing interactions and 3D geometries are not compatible with each other. This challenge has resulted in the failure of many ASO candidates and leaves important structured targets as inaccessible. The structure-based design disclosed herein solves this problem.

The instant structure-based design strategy has the potential to substantially improve therapeutic properties of ASOs. Although current FDA-approved ASOs are 18-30 nucleotides long, the design and preliminary binding study provide evidence that shorter ASOs may be able to achieve sufficient affinity and specificity by engaging in tertiary interactions with targets. A decrease in ASO size will allow more efficient delivery into cells, more favorable bio-distribution, and lower manufacturing costs.

### 1. Illustrative Studies

The instant disclosure provides general principles and method steps for structure-based ASO design, identified a structural design template, demonstrated the importance of tertiary interactions in this template, and generated a preliminary “vocabulary” of base triples as design building blocks. These results serve as the foundation for both aims.

2a. General Principles for Structure-Based ASO Design

These general principles are based on structural biology common sense and learning from existing RNA structures. These principles may be summarized as compatibility with target structures in 3D space and engineering in tertiary interactions.

Principle #1: Never design ASOs that pair with the whole hairpin loop. The practice of 1D design naturally leads to the desire to have ASOs complement the whole hairpin loop. However, not all base pairs will form due to 3D constraints. Base pairing in the hairpin stem requires the 5′- and 3′-most residues of the loop to be close to each other, whereas the helix formed between the hairpin loop and ASO is like a rigid rod and extends ˜30 Å per turn (11 bp). This is a structural biology common sense.

Principle #2: The helix formed by ASO and target RNA can be stabilized by base stacking with a hairpin stem. Base stacking is a major driving force for nucleic acids to achieve structural stability.

Principle #3: The affinity and specificity of an ASO for its target may be enhanced by base triples and non-canonical pairing interactions. These tertiary interactions are commonly seen in 3D structures of folded RNA molecules. Base triples may form between WC base pairs (between the ASO and hairpin loop) and “unpaired” loop residues. In a sense, these “loop triples” compensate the unpaired bases “wasted” due to the geometric constraints.

202b. A Design Template Derived from Pseudoknot Structures

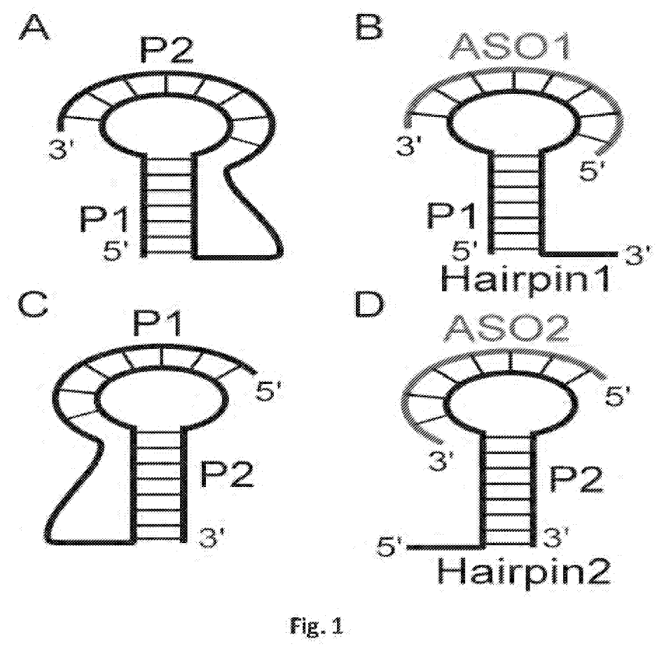

Artisans aim to understand how an ASO and a structured target RNA interact. However, the required structural information is lacking, i.e. few 3D structures of RNA hairpins in complex with ASOs are available. As disclosed herein, artisans can start out with inspecting RNA structures, such as pseudoknots, that resemble the association between ASOs and hairpins. Pseudoknot refers to a RNA hairpin with its loop pairing with an upstream or downstream strand, which may be viewed as equivalent to an ASO (FIG. 1A,B). In fact, each pseudoknot minimally contains two paired regions (P1 and P2). Either one may be viewed as the hairpin stem, whereas the other as the duplex formed between the hairpin loop and an ASO (FIG. 1A,C). Therefore, each pseudoknot structure teaches us two lessons about potential hairpin-ASO interactions (FIG. 1B,D).

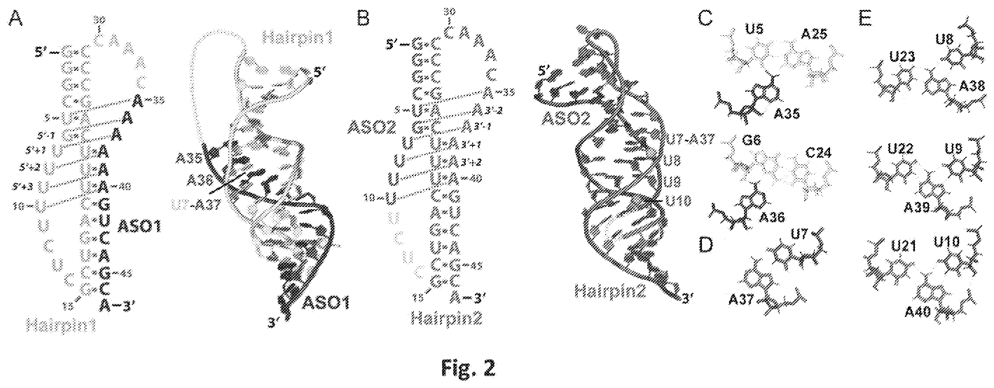

An illustrative embodiment of the invention inspected 93 pseudoknot structures and observed extensive tertiary interactions, including major-groove and minor-groove base triples (a third base contacting a base pair from the major- or minor-groove side respectively, see FIGS. 2E and 2C for examples), non-WC base pairs, and base stacking. Several pseudoknot structures were selected to design ASO-hairpin pairs and test their binding. Among them, the telomerase RNA pseudoknot structure stands out as having the most extensive tertiary interactions and the derived ASO-Hairpin pairs showed high affinity binding. Therefore, this structure was used as the template for subsequent biochemical characterization and ASO design work.

The telomerase RNA pseudoknot structure suggests two distinct ways ASOs can bind hairpin targets (FIG. 2A,B). ASO1 primarily pairs with 3′ region of the Hairpin1 loop (FIG. 2A). Two A residues overhang at the 5′-end of ASO1 (A35 and A36 in pseudoknot numbering), contacting the base pairs in the hairpin stem from the minor groove side (FIG. 2C). Such interactions are called A-minor interactions, a type of tertiary interactions that are important for RNA folding and are widely observed in RNAs such as group I introns and ribosomal RNAs (9, 10). ASO2 primarily pairs with 5′ region of the Hairpin2 loop (FIG. 2B). Three overhanging U residues at the 3′-end of ASO2 (U8-U10 in pseudoknot numbering) contacts the base pairs in the Hairpin2 stem from the major groove side (FIG. 2E). Even though these ASO residues are not involved in WC pairing, they nevertheless engage in extensive hydrogen bonds and the consecutive layers of base triples stack with each other. These tertiary interactions should contribute to binding affinity and structural specificity. As only some base combinations are compatible with base triples, it is expected that these ASO residues also help recognize target sequences they directly contact. Both types of “stem triples” are particularly useful for targeting hairpins with relatively short loops.

Two additional features are important for ASO design. First, both minor-groove and major-groove interactions are present in each ASO-hairpin pair. The minor-groove triples involving ASO1 5′ overhang residues (A35 and A36) (FIG. 2A) occur between 3′ loop residues in Hairpin2 and the duplex formed between ASO2 and Hairpin2 loop (FIG. 2B) Similarly, the major-groove triples involving the 3′ overhang (U8-U10) of ASO2 (FIG. 2B) are used by 5′ loop residues of Hairpin to engage the ASO1-Hairpin1 loop duplex (FIG. 2A). The second feature is that a Hoogsteen pair (between U7 and A37, pseudoknot numbering) bridges the double-layers of minor-groove triples and the triple-layers of major-groove triples (FIG. 2D). This Hoogsteen pair is important for proper geometry connecting the two neighboring segments.

52c. Biochemical Analyses Confirmed the Importance of Base Triples and Structural Recognition
i. Base Triple Interactions are Essential for High-Affinity Binding

Base triples in the ASO-hairpin pairs were disrupted and their affinities then measured using isothermal titration calorimetry (ITC) (FIG. 3A,B,C,D). The K_{d} values are 70±10 nM for ASO1/Hairpin1 and 28±4 nM for ASO2/Hairpin2 (FIG. 3C,D). Deletion of the two 5′ A overhangs reduces ASO1 binding to Hairpin1 by 8 fold, indicating the importance of the minor-groove triples (FIG. 3A). Mutation of the three U residues at the 5′ end of Hairpin1 loop to C's reduced binding by >10 fold, highlighting the critical contribution of the major-groove triples to ASO1-Hairpin1 binding. Truncation of the three U overhanging residues in ASO2 reduces binding to Hairpin2 by 20 fold (FIG. 3B). Therefore, both substitution (in Hairpin1) and truncation (in ASO2) demonstrate the importance of the major-groove base triples. Dramatically, substituting A36 and A37 in the Hairpin2 loop with two C residues disrupts the ASO2 binding by 130 fold. This result confirms the importance of the minor-groove base triples, and suggests that the minor-groove triples contribute more when they occur in the hairpin loop. The mutagenesis results for the two hairpin-ASO pairs suggest that the base triples observed in the pseudoknot structure are likely to be preserved in the ASO-hairpin complexes.

ii. Hairpin Structures are Important for ASO Target Binding and Specificity

The 3D structure (FIG. 2A) suggests that the tertiary interactions with ASOs require the target RNA to be in a hairpin conformation. It was tested if a stable hairpin stem is important for their interaction by truncating the Hairpin1 stem from 6 base pairs to 4 (FIG. 3A). Strikingly, no binding could be detected between ASO1 and the truncated Hairpin1. This result suggests that ASO1 recognizes the Hairpin1 structure even though it does not directly contact the deleted residues. Therefore, in a structure-based ASO design, tertiary interactions and hairpin structures all contribute to the target binding specificity.

2d. Backbone Modifications

Backbone modifications in ASOs increase their in vivo stability, enhance target affinity, and reduce toxicity. The ASOs designed will contain the commonly used modifications (FIG. 4).

Phosphorothioate (PS) is a backbone modification in which a non-bridging oxygen atom of a phosphodiester is substituted by sulfur. This modification substantially improves resistance to nucleases and enhances the binding of ASOs to serum proteins, which is beneficial for delivery. This modification does not dramatically change nucleic acids geometry. The PS modification introduces only subtle changes in backbone geometry, and thereby is expected to be well tolerated at all ASO positions.

Ribose modifications 2′-O-methoxyethyl (2′-MOE) and 2′-O-methyl (2′-OMe) are used in second-generation of RNA/DNA-based ASOs. These modifications increase the hybridization affinity for targets, enhance nuclease resistance, and reduce immunostimulatory activity. Compared to native RNAs, 2′-OMe and 2′-MOE make the 2′-functional group bulkier. In the 3D structure, 2′-OH are open to the solvent at most ASO positions and therefore 2′-MOE and 2′-OMe should are well tolerated. The only exceptions are residues 4-7 in ASO2 (FIG. 4B), the 2′-OH of which are located in relatively crowded surroundings. For these positions, 2′-OMe should be fine, but not 2′-MOE. Certain embodiments can include 2′-fluoro modification at these positions as a another option.

Phosphorodiamidate morpholino oligomers (PMO) are nucleic acid analogs that contain 6-member morpholino ring with phosphorodiamidate linkage (FIG. 4) (11). The PMO backbone is highly resistant to nucleases. Another feature of PMO backbone is the lack of negative charges, which increases affinity for targets because there is less charge-charge repulsion to overcome. PMO has the same number of covalent linkages along its backbone as that in RNA. The morpholino moiety has the same number of non-hydrogen atoms as the ribose, with an extra atom in the ring but lacking the 2′-OH. The dimethylamine group on the phosphate is bulkier than the single non-bridging oxygen in a native nucleic acid, but should be well accommodated at all ASO positions.

Locked nucleic acid (LNA) has a methylene bridge covalently linking 2′-0 and 4′-C (FIG. 4) (12). The bridge locks the ribose in a conformation found in A-form helix (13-15) and hence enhances the target affinity. For reasons explained above for 2′-OMe, it is expected that LNA is compatible with the disclosed ASO designs.

Peptide nucleic acid (PNA) analogs have nucleobases as side chains and peptide-bond-linked N-(2-aminoethyl)glycine units as backbone (16). PNA ASOs are very resistant to both nuclease and protease degradation. Similar to PMO, PNA has neutral backbone and binds target with higher affinity than DNA or RNA. Previously determined structures showed that PNA and complementary RNA hybridize to adopt an A-form helical conformation with the PNA chain being more flexible (17, 18). It is possible that PNA can fit in the design templates.

### 102. Duchenne Muscular Dystrophy

Duchenne muscular dystrophy (DMD) is a rare genetic disorder that is characterized by progressive muscle weakening and wasting, almost exclusively affecting boys. According to the NIH Genetic and Rare Diseases (GARD) Information Center, DMD affects approximately 1 in 3,500 to 5,000 male births worldwide. There is no cure. Physical therapy and hormonal medications are used to control symptoms and improve quality of life. Even with improved care in recent years, patients become wheelchair-bound in their early teens and have much shorter life span, typically dying in their 30s.

DMD is caused mutations in the gene encoding a protein called Dystrophin, which supports muscle fiber strength. In the past two years, two ASO drugs have been approved by the US FDA for treating DMD.

3a. Eteplirsen and Golodirsen

Eteplirsen and golodirsen were developed by Sarepta Therapeutics to treat specific types of DMD that are caused by mutations that change the reading frame of Dystrophin translation. About 13% and 8% of DMD patients fall in these categories. Eteplirsen is a 30-nucleotide ASO that binds to exon 51 and causes it to be skipped during pre-mRNA splicing and thereby restore proper translation reading frame. This “exon skipping therapy” produces a Dystrophin protein that is partially functional and reduces the severity of the disease. Golodirsen is a 25-nucleotide ASO that binds and causes exon 53 to be skipped.

The accelerated approval of Eteplirsen in 2016 was both exciting and controversial. The drug was able to partially restore Dystrophin protein production. As the first in its class, it proved that the novel exon skipping mechanism works. However, eteplirsen is only marginally effective clinically and does not show significant improvement in the standard walking test. Two FDA review panel members resigned in protest. In 2018, the European Medicines Agency refused to approve it. Furthermore, eteplirsen is expensive, costing $300,000 annually per person.

3b. The Structure-Based Designs

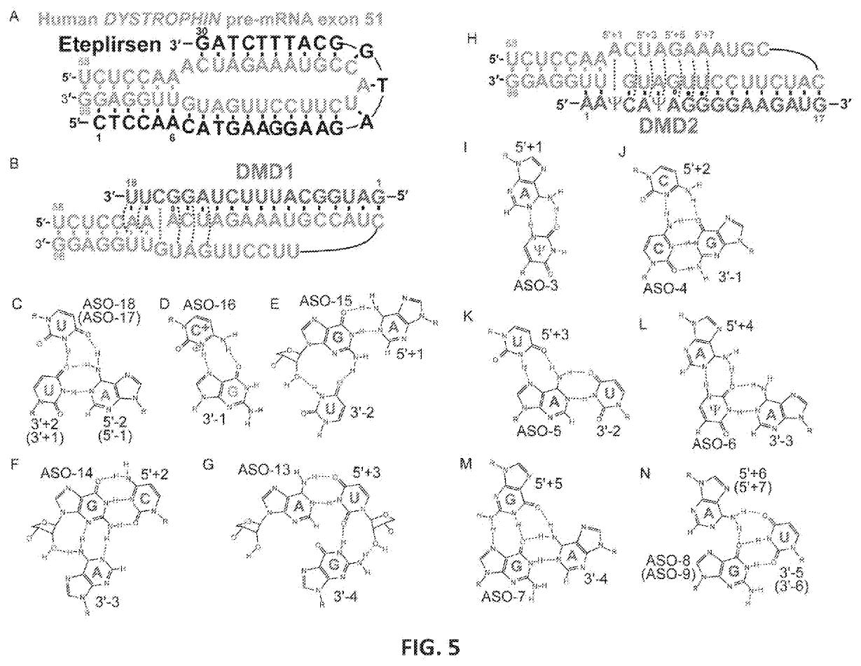

The structure-based design method makes it possible to greatly improve eteplirsen, golodirsen, and ASO drug candidates at various stages of development. Here eteplirsen is used as an example. The eteplirsen-binding site and surrounding sequences on exon 51 fold to a hairpin structure that is predicted with high confidence (FIG. 5A). The base pairing between eteplirsen and its target is structurally incompatible with the hairpin conformation, i.e. eteplirsen has to break the base pairs in the hairpin before it can bind fully with exon The energy it spends to do so will reduce its binding affinity and specificity.

Two ASOs (DMD1 and DMD2) were designed that are not only compatible with the hairpin structure, but also forge extensive additional interactions (FIG. 5B-N). DMD1 and DMD2 bind to 5′ and 3′ regions of the hairpin loop (FIG. 5B,H), respectively. They both form many hydrogen bonds with the target RNA (FIG. 5C-G, 5I-N), more than those found in WC base pairs. The free energies gained from these additional interactions and saved from not having to disrupt the hairpin stem should allow the ASOs to bind to the target RNA with high affinity and specificity even though they are shorter (18 and 17 nucleotides for DMD1 and DMD2 respectively). The shorter sequences come with great benefits including easier delivery, lower costs of synthesis, and lower chance of triggering innate immunity.

The ASO designs have features that position them favorably in obtaining intellectual properties. In addition to the relatively short length, the ASOs are not fully complementary in sequence to their targets due to non-canonical pairs designed to optimize geometric compatibility and tertiary interactions (FIG. 5B,H). Naturally occurring modified bases such as pseudouridine (ψ) can also be included.

3. ASOs that Inhibit the SARS-CoV-2 Virus (See Also Li et al., Structure-Based Design of Antisense Oligonucleotides that Inhibit SARS-CoV-2 Replication. bioRxiv [Preprint]. 2021 Aug. 24:2021.08.23.457434. doi: 10.1101/2021.08.23.457434. PMID: 34462746; PMCID: PMC8404888)

Coronavirus disease 2019 (COVID-19) is an infectious disease caused by the newly discovered severe acute respiratory syndrome-related coronavirus 2 (SARS-CoV-2). Since the outbreak, it has quickly developed into a serious global pandemic because of its lethality and contagiousness. As of May 27, there have been 5.6 million confirmed cases with over 350,000 deaths worldwide. The daily confirmed new case and death counts stand at around 100,000 and 1,500 respectively. Common symptoms include fever, body ache, dry cough, fatigue, chills, headache, sore throat, and loss of smell. In severe cases, high fever, severe cough, and shortness of breath occur. Some patients also experience neurological and/or gastrointestinal symptoms or have an increased risk of stroke. A major difficulty in containing SARS-CoV-2 is that some 42% infected people are asymptomatic but can infect other people (19). SARS-CoV-2 is mainly spread from person to person, via droplets, aerosols, and contact with infected surfaces. It is unclear when an effective vaccine will become available. Remdesivir is the only drug that has received authorization for treating COVID-19, but its benefits are marginal.

There have been efforts to develop ASO and other synthetic oligonucleotide drugs targeting viruses that cause respiratory diseases (20). The viruses that have been previously targeted include SARS-CoV, influenza A, respiratory syncytial virus (RSV), and adenovirus. The most advanced one is a small interfering RNAs (siRNAs) called RSV01 that is developed by Alsylam for treating RSV infections among lung-transplant patients (21). Encouragingly, their phase IIb trial showed significant efficacy in preventing RSV infections in 3-6 months. This clinical trial is also interesting in that the siRNA is delivered via nasal sprays, making it very easy to administer. As siRNAs are also short synthetic oligonucleotides, the ASO drug candidates will be administered either as a nasal spray or intravenously The convenience of the nasal spray formulation is particularly attractive, as it will make the drug more accessible to the underserved/disadvantaged populations and allow administration outside of the controlled healthcare settings required for IV delivery siRNAs are double-stranded RNAs that need to be unwound and the guide strand has to be incorporated into a cellular effector protein called Argonaute before becoming active. The ASO route is preferred in part because the backbone modifications necessary for nuclease resistance interfere with Argonaute-siRNA interaction and thereby are limited for siRNAs.

SARS-CoV-2 is a positive-sense, single-stranded RNA virus with a very large genome, which makes particularly susceptible to ASO drugs. Fifteen years ago, Neuman et al. designed nine ASOs targeting SARS-CoV RNA and found that several were effective to variable degrees in inhibiting the virus in cultured cells (22). The most effective one (called TRS2 as shown in FIG. 6A) inhibits the virus at 10-20 μM and binds to the transcription regulatory sequence (TRS). The TRS region is predicted to fold into a hairpin and is essentially identical between SARS-CoV and SARS-CoV-2. Therefore, ASOs targeting the TRS should similarly inhibit SARS-CoV-2. However, the 21-nt TRS2 is complementary to nearly the entire hairpin loop. The A-form helix they form is expected to extend over 55 Å, making it impossible for the neighboring residues at the top of the hairpin stem to pair. As a consequence, either not all TRS2 bases can hybridize to the target, or they will at the free-energy expense of having to disrupt the base pairing in the hairpin stem. In either case, TRS2 cannot achieve the intended binding affinity and specificity. Any unpaired TRS2 residues will only contribute to non-specific binding.

Four ASOs were designed that are spatially compatible with the SARS-CoV-2 TRS hairpin and harness the power of tertiary interactions to maximize binding affinity and specificity. SBD1 shares fourteen nucleotides with TRS2, which form W-C base pairs with 5′ region of the loop, leaving nine 3′-loop residues for connecting back to the hairpin stem (FIG. 6B). Four residues (3′-CTTT (SEQ ID NO: 27)) were also included that form major groove base triples with the hairpin stem base pairs and a Hoogsteen pair with the 3′ terminal (3′−1) A residue of the loop (FIG. 6C-F). An additional A residue at the 3′ loop region (3′−2) can form a minor-groove base triple with the SBD1-loop duplex (FIG. 6G). These base triple and Hoogsteen pairing interactions are accompanied by strong base stacking that further stabilizes the ASO-hairpin complex. These extensive tertiary interactions in compatible geometry give us strong hope that the SBD1 will bind SARS-CoV-2 RNA with higher affinity and specificity

SBD2 hybridizes primarily to twelve residues in the 3′ region of the TRS hairpin loop (FIG. 6I). In this design, seven 5′ loop residues (from 5′+2 to 5′+8) can form consecutive layers of major-groove base triples (FIG. 6K-P). The whopping consecutive layers of base triples give us high hope that 15 nucleotides are sufficient for this ASO to achieve sufficient affinity and specificity. Another innovative aspect of this design is introduction of a non-WC pair at position 5. Additionally, two A residues were added to the 5′ end of SBD2 to bind to the hairpin stem via A-minor interactions, and a third residue G to form the Hoogsteen pair with the C at the 5′+1 loop position (FIG. 6J).

SBD1 and SBD2 are PMO oligos, which lack the negative charges in most nucleic acids backbones. Their association with target RNAs does not have to overcome charge-charge repulsion. On the other hand, PMO oligos are only available from one company, Gene Tools, and the bases they provide are limited to A, T, C, and G.

Three additional ASOs were designed, SBD3, SBD4 and SBD5, that resemble SBD1 and SBD2 but with PS and 2′-MOE (or the similar 2′-OMe) modifications (FIG. 6Q,R). This backbone configuration is the same as four approved ASO drugs. Additionally, to optimally accommodate the bridging Hoogsteen pair and major-groove base triples in SBD4, pseudouridin is used at positions 4, 6, 11, and 12. SBD5 is an alternative of SBD4, with ψ at position 3. The resulting C-ψ Hoogsteen pair does not require C to be protonated as in the C-G Hoogsteen pair in the SBD4-target complex.

The disclosure provided herein facilitates production of drugs that can both prevent and treat COVID-19. Such drugs may be administered as a nasal spray or via intravenous injection. The nasal spray formulation is particularly attractive because it can be easily given to people who are at high risk, do not readily have access to healthcare, or just start to show symptoms.

### EXAMPLE 1 REFERENCES

1. Bennett C F. 2019 Therapeutic antisense oligonucleotides are coming of age. Annu. Rev. Med. 70: 307-21
2. Stein C A, Castanotto D. 2017. FDA-Approved Oligonucleotide Therapies in 2017. Mol. Ther. 25: 1069-75
3. Crooke S T, Witztum J L, Bennett C F, Baker B F. 2018. RNA-Targeted Therapeutics. Cell Metab. 27: 714-39
4. Zamecnik P C, Stephenson M L. 1978. Inhibition of Rous sarcoma virus replication and cell transformation by a specific oligodeoxynucleotide. Proc Natl Acad Sci USA 75: 280-4
5. Stephenson M L, Zamecnik P C. 1978. Inhibition of Rous sarcoma viral RNA translation by a specific oligodeoxyribonucleotide. Proc Natl Acad Sci USA 75: 285-8
6. Kuijper E C, Bergsma A J, Pim Pijnappel W W M, Aartsma-Rus A. 2020. Opportunities and challenges for antisense oligonucleotide therapies. J. Inherit. Metab. Dis.
7. Bennett C F, Krainer A R, Cleveland D W. 2019. Antisense Oligonucleotide Therapies for Neurodegenerative Diseases. Annu. Rev. Neurosci. 42: 385-406
8. Dominguez D. Freese P. Alexis M S, Su A, Hochman M, Palden T, Bazile C, Lambert N J, Van Nostrand E L, Pratt G A, Yeo G W, Graveley B R, Burge C B. 2018. Sequence, Structure, and Context Preferences of Human RNA Binding Proteins. Mol. Cell 70: 854-67 e9
9. Cate J H, Gooding A R, Podell E, Zhou K, Golden B L, Szewczak A A, Kundrot C E, Cech T R, Doudna J A. 1996. RNA tertiary structure mediation by adenosine platforms. Science 273: 1696-9
10. Nissen P, Ippolito J A, Ban N, Moore P B, Steitz T A. 2001. RNA tertiary interactions in the large ribosomal subunit: the A-minor motif. Proc. Natl. Acad. Sci. U.S.A. 98: 4899-903
11. Nan Y. Zhang Y J. 2018. Antisense Phosphorodiamidate Morpholino Oligomers as Novel Antiviral Compounds. Front. Microbiol. 9: 750
12. Hagedorn P H, Persson R. Funder E D, Albaek N, Diemer S L, Hansen D J, Moller M R, Papargyri N, Christiansen H, Hansen B R, Hansen H F, Jensen M A, Koch T. 2018. Locked nucleic acid: modality, diversity, and drug discovery. Drug Discov. Today 23: 101-14
13. Nielsen K E, Rasmussen J, Kumar R., Wengel J. Jacobsen J P, Petersen M. 2004. NMR studies of fully modified locked nucleic acid (LNA) hybrids: solution structure of an LNA:RNA hybrid and characterization of an LNA:DNA hybrid. Bioconjugate chemistry 15: 449-57
14. Eichert A, Behling K, Betzel C, Erdmann V A, Furste J P, Forster C. 2010. The crystal structure of an ‘All Locked’ nucleic acid duplex. Nucleic Acids Res. 38: 6729-36
15. Petersen M, Bondensgaard K, Wengel J, Jacobsen J P. 2002. Locked nucleic acid (LNA) recognition of RNA: NMR solution structures of LNA:RNA hybrids. J. Am. Chem. Soc. 124: 5974-82
16. Quijano E, Bahal R, Ricciardi A, Saltzman W M, Glazer P M. 2017. Therapeutic Peptide Nucleic Acids: Principles, Limitations, and Opportunities. The Yale journal of biology and medicine 90: 583-98
17. Kiliszek A, Banaszak K, Dauter Z, Rypniewski W. 2016. The first crystal structures of RNA-PNA duplexes and a PNA-PNA duplex containing mismatches—toward anti-sense therapy against TREDs. Nucleic Acids Res. 44: 1937-43
18. Brown S C, Thomson S A, Veal J M, Davis D G. 1994. NMR solution structure of a peptide nucleic acid complexed with RNA. Science 265: 777-80
19. Yang R, Gui X, Xiong Y. 2020. Comparison of clinical characteristics of patients with asymptomatic vs symptomatic coronavirus disease 2019 in Wuhan, China. JAMA Netw Open 3: e2010182
20. Asha K, Kumar P, Sanicas M, Meseko C A, Khanna M, Kumar B. 2018. Advancements in nucleic acid based therapeutics against respiratory viral infections. J. Clin. Med. 8
21. Gottlieb J, Zamora M R. Hodges T, Musk A W, Sommerwerk U, Dilling D, Arcasoy S, DeVincenzo J, Karsten V, Shah S, Bettencourt B R, Cehelsky J, Nochur S, Gollob J, Vaishnaw A, Simon A R, Glanville A R. 2016. ALN-RSV01 for prevention of bronchiolitis obliterans syndrome after respiratory syncytial virus infection in lung transplant recipients. J. Heart Lung Transplant. 35: 213-21
22. Neuman B W, Stein D A, Kroeker A D, Churchill M J, Kim A M, Kuhn P, Dawson P, Moulton H M, Bestwick R K, Iversen P L, Buchmeier M J. 2005. Inhibition, escape, and attenuated growth of severe acute respiratory syndrome coronavirus treated with antisense morpholino oligomers. J. Virol. 79: 9665-76

### Example 2: Antisense Oligonucleotides for Treating Coronavirus Infections

As discussed in detail in Li et al., Structure-based design of antisense oligonucleotides that inhibit SARS-CoV-2 replication. bioRxiv [Preprint]. 2021 Aug. 24:2021.08.23.457434. doi: 10.1101/2021.08.23.457434. PMID: 34462746; PMCID: PMC8404888; that is incorporated hereon by reference, antisense oligonucleotide (ASO) sequences have been identified that inhibit the novel coronavirus severe acute respiratory syndrome-related coronavirus-2 (SARS-CoV-2).

The text below briefly describes the method and elaborates on two designs and the results, ones that demonstrate the anti-SARS-CoV-2 activity for these sequences.

### Brief Description of Structure-Based ASO Design Method

Conventional ASO designs strictly follow the rule of Watson-Crick (WC) base pairing along linear target sequences. However, viral RNAs often fold into strong secondary and tertiary structures that interfere with ASO hybridization. Conventional ASO design strategies do not adequately address this problem. The method takes advantage of target RNA structures and use structure-based ASO designs to target SARS-CoV-2. The designs are compatible with target structures in three-dimensional (3D) space and engage in tertiary interactions in addition to WC base pairing. The “3D-ASOs” designed this way bind targets with enhanced affinity due to the tertiary interactions gained and the free energy saved for not having to disrupt target structures. Moreover, binding specificity is elevated because 3D-ASOs not only recognize target sequences but also the shapes and hydrogen-bonding patterns.

SARS-CoV-2 is a positive-sense, single-stranded RNA virus with a large genome, which makes it particularly susceptible to ASO drugs. The 3D-ASOs target the frameshift stimulation element (FSE) and transcription regulatory sequence (TRS), respectively.

PRF3pPMO that Targets the FSE

Coronaviruses require programmed −1 ribosomal frameshifting (−1 PRF) to translate the ORF1b, which encodes the RNA-dependent RNA polymerase, which is the catalytic subunit of the replicase and transcriptase complexes. Therefore, the PRF is absolutely essential for viral replication. The FSE is located at the junction between ORF1a and ORF1b. It contains a pseudoknot structure proceeded by a slippery sequence and an attenuator hairpin. During translation, a ribosome is stalled at the slippery site by the pseudoknot and this process gives the ribosome a chance to slip back by one nucleotide before being able to disrupt the pseudoknot and continue translating. Without slipping, the ribosome encounters a downstream stop codon and fall off, producing protein 1a. With slipping, it completes the translation of ORF1b, generating protein 1a-1b.

A 3D-ASO was designed that disrupts the pseudoknot structure with the goal of eliminating PRF. This pseudoknot contains stems 1-3 (FIG. 7A). The idea is to disrupt a part of the structure so that the ribosome will stall or will not stall at the slippery site. This ASO, is called PRF3pPMO, is shown in FIG. 7B. This 22-nt PMO oligo hybridizes to a region 3′ to stem 3 and engages in three major-groove, five minor-groove base triples and a Hoogsteen pair in-between.

SBD1 that Targets the TRS

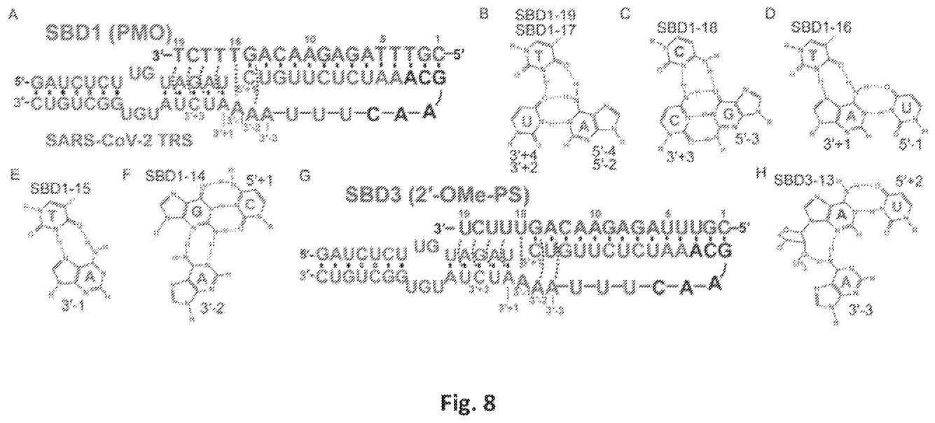

SBD1 form fourteen W-C base pairs with 5′ region of the target hairpin loop, leaving nine 3′-loop residues for connecting back to the hairpin stem (FIG. 8A). It also contains five residues (3′-TCTTT (SEQ ID NO: 25)) that form major groove base triples with the hairpin stem base pairs and a Hoogsteen pair with the 3′ terminal (3′−1) A residue of the loop (FIG. 8B-E). An additional A residue at the 3′ loop region (3′−2) can form a minor-groove base triple with the SBD1-loop duplex (FIG. 8F). These base triple and Hoogsteen pairing interactions are accompanied by strong base stacking that further stabilizes the ASO-hairpin complex.

Identification of Lead ASOs that Inhibit SARS-CoV-2 in Cultured Human Cells

Synthetic ASOs were generated with a PMO backbone and tested their abilities to inhibit SARS-CoV-2 replication in cultured HEK293-hACE2 cells. All experiments involving live viruses are conducted in a UCLA BSL3 high-containment facility. There are established cellular models for SARS-CoV-2 infection. SARS-CoV-2 (isolate USA-WA1/2020) has been obtained from BEI Resources of NIAID. SARS-CoV-2 is passaged once in Vero E6 cells and viral stocks are aliquoted and stored at −80° C.

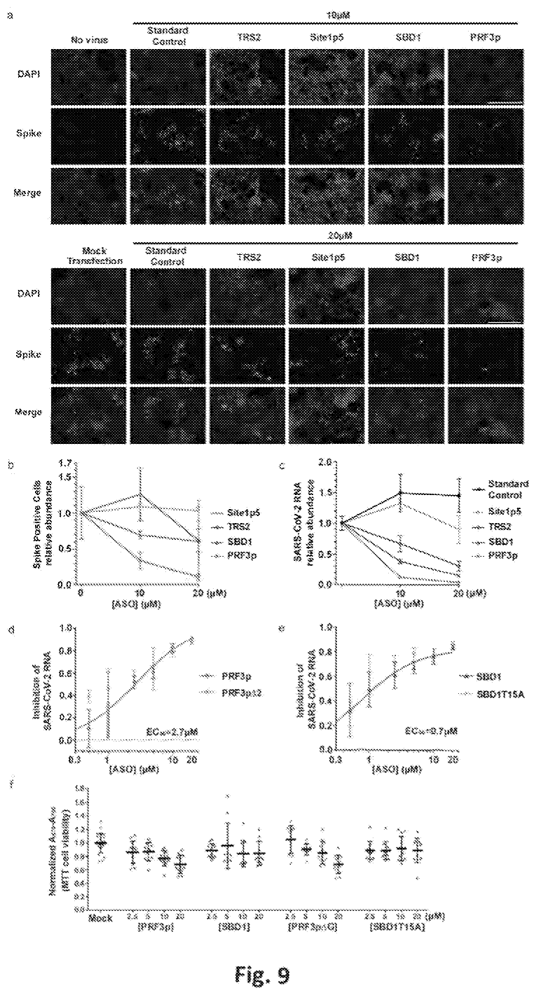

To evaluate the effects of ASOs on SARS-CoV-2 replication, HEK293 cells expressing the human angiotensin-converting enzyme 2 (hACE2) receptor were transfected with ASOs. The following day, viral inoculum was added to the transfected cells in serum-free medium at a multiplicity of infection (MOI) MOI of 0.1. After 1 h of incubation, the inoculum was replaced with serum supplemented media. At 24 h post infection (hpi), the cells were fixed with methanol to inactivate the virus and examined viral infection using immunocytochemistry (ICC) with an antibody specific for SARS-CoV-2 spike protein. SARS-CoV-2 spike antigen can be detected in infected cells with mock transfection (without ASO), confirming active viral infection (FIG. 9A). It was observed that PRF3pPMO and SBD1 showed strong inhibition, with viral signals below detectable level on the ICC images. Site1p5PMO is a 24mer PMO ASO conventionally designed to be completely complementary to SARS-CoV-2 TRS and surrounding regions. Site1p5PMO showed little inhibition of the virus. A previously reported ASO, PREV, showed mild inhibition of the virus.

To confirm the SARS-CoV-2 inhibition activity of the 3D-ASOs, total RNAs were extracted from the infected cells and quantified viral and host RNAs using qRT-PCR. Consistent with the ICC results, PRF3pPMO strongly inhibited SARS-CoV-2 replication, with 14% (10 μM ASO) and 4% (20 μM ASO) of viral RNAs detected relatively to the non-specific Standard Control PMO after 24 hpi (FIG. 9B). At 10 μM and 20 μM concentrations, SBD1 reduced the viral RNA to 44% and 17% that of the Standard Control, respectively. These results are highly significant statistically. The qRT-PCR data confirmed the mild inhibition activity of PREV and the lack of activity for Site1p5PMO. Taken together, lead ASO candidates with SARS-CoV-2 antiviral activity have been identified for additional preclinical safety and toxicity studies.

### Extension of the 3D-ASO Designs

PRF3pPMO and SBD1 are PMOs. Because the way 3D-ASOs are designed, one can reasonably expect that similar ASOs with alternative backbone may be active in inhibiting SARS-CoV-2. Artisans can further determine how these modifications affect the binding of the lead 3D-ASOs to their target.

A gapmer version of PRF3pPMO called PRF3pGapmer (FIG. 7C) was designed. Gapmers contain DNA residues at an internal region of an ASO that induces the degradation of the target RNA by RNase H in addition to disrupting the pseudoknot. Residues 4-13 in PRF3pGapmer are DNA residues. Residues 1-3 and 14-22 contain the 2′-O-methyl (2′-OMe) modification. PRF3pGapmer has phosphorothioate (PS) modification throughout its sequence.

SBD3 resembles SBD1 but with PS and 2′-OMe or the similar 2′-O-methoxyethyl modifications (FIG. 8G). This backbone configuration is used by four FDA-approved ASO drugs. SBD3 will have two hybrid versions, each with either 2′-OMe or 2′-F modifications at the crowded residues 4-7 region. These hybrid oligos maximize the chance of 2′-MOE being usable, since there have been reports that 2′-MOE can be more effective than 2′-OMe in triggering exon skipping and induce fewer proinflammatory effects. Due to its backbone, SBD3 can form an additional minor groove base triple as shown in FIG. 8H. 3D-ASOs similar to PRF3pPMO, PRF3pGapmer, SBD1, and SBD3 will be synthesized with LNA and PNA modifications in their sequences.

### Example 3: Design of ASOS that Upregulate the Expression of Human FAN1

This example, illustrates embodiments of the invention targeting FAN1, a Fanconi-associated nuclease 1.

FAN1 is also called KIAA1018, MTMR15, FANCD2/FANC1-associated nuclease 1. It is required for DNA interstrand crosslink repair, but is not involved in DNA double-strand break resection. FAN1 is recruited to DNA damage sites by monoubiquitinated FANCD2. FAN1 has a UBZ-type ubiquitin-binding domain at the N-terminus, a SAP-type DNA binding domain in the middle, and a nuclease domain termed the “VRR_nuc” domain at the C-terminus. It has endonuclease activity toward 5′ flaps and 5′-3′ exonuclease activities, which require the VRR_nuc domain.

The FAN1 protein is expressed in two major isoforms, as indicated in the UniProt database. Isoform 1 is the full length containing 1017 a.a. and all the functional domains. Isoform 2 contains 533 a.a., with residues 1-526 identical to isoform 1 and residues 527-533 changed from AKALAGQ in isoform 1 to FCWLLLQ. Isoform 2 misses residues 534-1017 in the full-length protein, including the VRR-nuc domain, and thus is not expected to be functional. Therefore, one will be able to increase the functional FAN1 expression level by diverting the mRNAs for isoform 2 to the isoform 1 during RNA processing. Seven additional isoforms are computationally predicted, containing 430, 237, 573, 318, 587, 103, and 298 a.a. in length.

It was observed that the mRNA isoforms coding for the shorter protein isoform are generated by alternative polyadenylation, not by alternative splicing. A search of the human protein atlas found the following five mRNA variants;

FAN1-201 (ENST00000362065.8) encodes 1017 a.a., containing 15 exons (13 coding) and 4,891 nt in length. The lengths (nt) of exons and introns are noted below, with the numbers in parenthesis the indicating lengths of introns. 139(500)1,386(2,220)141(2,355)202(3,042)234 . . . (4306)1,543. The last exon (1,543 nt) contains multiple copies of polyadenylation signal (PAS) sequences.

FAN1-202 (ENST00000561594.5) encodes 533 a.a. (isoform 2), containing 4 exons (3 coding) and 2,738 nt in length. 36(626)1,386(2,220)141(2,355)1,175. Exon 4 has 1,175 nt, which includes the 202 nt of exon 4 and 973 nt of intron 4-5 in

FAN1-201. The coding extends 22 nt before hitting the TAA (SEQ ID NO: 14) stop codon. It does not include 3′ region of intron 4-5 or exon 5 of FAN1-201. Importantly, residues 1149-1154 of exon 4 contains a PAS (AUUAAA, (SEQ ID NO: 15) PAS1) that is about 20 nt from the 3′ end (the cleavage site), indicating that FAN1-202 is generated via alternative polyadenylation.

FAN1-203 (ENST00000561607.5) encodes the 533-residue isoform 2, containing 4 exons (3 coding) and 2,690 nt in length.

133(487)1,386(2,220)141(2,355)1,030. Exon 4 contains 1,030 nt, which is identical to exon 4 of FAN1-202 except being shorter. Importantly, resides 995-1000 of exon 4 contains a PAS (AAUAAA, (SEQ ID NO: 16) PAS2) that is about 30 nt from the 3′ end, indicating that FAN1-203 is generated by alternative polyadenylation at this site. Note that this PAS is also present in FAN1-202.

FAN1-205 (ENST00000562892) encodes a 105-a.a. protein, containing 3 exons (all coding) and 344 nt in length. 81(3,527)141(2,355)122. Exon 3 contains 122 nt that is identical to exon 4 of FAN1-201, -202, and -203 variants except being shorter. This variant does not have a stop codon (terminating in the coding region) and appears to be incomplete in the 3′ end.

FAN1-207 (ENST00000565466) encodes the 533-a.a. protein, containing 4 exons (3 coding) and 2,304 nt in length. 119(500)1,386(2,220)141(2,355)658. Exon 4 contains 658 nt that is identical to exon 4 of FAN1-202 and -203 except being shorter. It is not clear what PAS sequence directs the alternative adenylation at this site. In sum, production of transcripts FAN1-202 and FAN1-203 are clearly directed by two alternative PAS sequences, providing an opportunity for drug discovery.

A recent paper (Naveed et al. “NEAT1 polyA-modulating antisense oligonucleotides reveal opposing functions for both long non-coding RNA isoforms in neuroblastoma” Cellular and Molecular Life Sciences 2020) reports that ASOs can be used to inhibit polyadenylation of shorter transcript and increase the production of longer RNAs. Following this paper, four ASO sequences were designed that potentially modulate polyadenylation of FAN1 and effectively the full-length protein expression.
3′-AAAUUACAAAUGAACGAAUAUUUUAC-5′ (SEQ ID NO: 31) (target PAS1 exon 4 res. 1154-1129)
5′-UUUAAUGUUUACUUGCUUAUAAAAUG-3′ (SEQ ID NO: 32) (FAN1ASO1, 26 nt, 13 U, 8 A, 5 GC)
3′-GUCGGUCUCCCACCGAUUUAAAUUA-S′ (SEQ ID NO: 33) (target PAS1 exon 4 res. 1173-1149)
5′-CAGCCAGAGGGUGGCUAAAUUUAAU-3′ (SEQ ID NO: 34) (FAN1ASO2, 25 nt, well-balanced seq)
3′-AAAUAACUUCAUGAAAAAUUUUUAG-5′ (SEQ ID NO: 35) (target PAS2 exon 4 res. 1000-976)
5′-UUUAUUGAAGUACUUUUUAAAAAUC-3′ (SEQ ID NO: 36) (FAN1ASO3, 25 nt, 12 U, 9A, 4 GC)
3′-GUUAAUUAAUGUUCAUACAUUAAAUAA-5′ (SEQ ID NO: 37) (target PAS2 exon 4 res. 1021-995)
5′-CAAUUAAUUACAAGUAUGUAAUUUAUU-3′ (SEQ ID NO: 38) (FAN1ASO4, 27 nt, 12 U, 11 A, 4GC)

Secondary structure prediction for the exon 4 of both FAN1-202 and FAN-203 mRNAs were performed using the RNAstructure online server which found both PAS sequences to be present as part of high-confidence hairpin structures. Residues 1105-1155 for a hairpin that is present in all top 20 predictions. The PAS sequence (AUUAAA, (SEQ ID NO: 15)) is present at the 3′ end (residue 1149-1154).

Residues 993-1022 form hairpin that appears in the top 19 predictions for FAN1-203 exon 4, as well as in 19 out of the top 20 predictions for the longer FAN1-202 exon 4. The PAS (AAUAA, (SEQ ID NO: 40)) is located at the 5′ end (residues 995-1000).

These structures can potentially sequester the PAS' and prevent access by the mRNA cleavage and polyadenylation machinery. This observation may explain why these PAS sites are only used a fraction of time. Several ASO sequences that target these structures and their flanking regions have been designed (FIG. 26).

### Example 4: Design 3D-ASOs to Target Pri-Mir-3915 in Intron 13-14 of Human Dystrophin Pre-MRNAs

This example illustrates embodiments of the invention targeting a putative pri-miRNA hairpin (pri-miR-3915, genome coordinate X32,583,656-32,583,752) in intron 13-14 (between exons 13 and 14) of human Dystrophin pre-mRNAs. Pri-miRNAs have been shown to serve as transcription termination signals¹. It is hypothesized that cleavage of the MIR3915 hairpin by pri-miRNA processing machinery, the Microprocessor Complex, terminates DYSTROPHIN transcription and thereby is a mechanism for regulating Dystrophin expression. Consistent with this idea, two DMD transcripts DMD-223 and DMD-214 end shortly downstream of pri-miR-3915. DMD-214 and DMD-223 are not expected to produce functional Dystrophin proteins. Without being bound by a specific theory or mechanism of action, this mechanism suggests that 3D-ASOs can be developed to reduce the transcription termination by inhibiting pri-miR-3915 cleavage by the Microprocessor Complex, reduces the premature transcription termination, and thus increases the production of longer and functional Dystrophin proteins.

To design 3D-Asos, a performed secondary structure prediction of pri-miR-3915 was first performed as shown in FIGS. 27C and 27D.

### 3D-ASO Designs

See FIG. 27D.

### EXAMPLE 4 REFERENCES

1. Dhir, A., Dhir, S., Proudfoot, N. J. & Jopling, C. L. Microprocessor mediates transcriptional termination of long noncoding RNA transcripts hosting microRNAs. Nat. Struct. Mol. Biol. 22, 319-327 (2015).

All publications mentioned herein (e.g. Li et al., Structure-based design of antisense oligonucleotides that inhibit SARS-CoV-2 replication. bioRxiv [Preprint]. 2021 Aug. 24:2021.08.23.457434. doi: 10.1101/2021.08.23.457434. PMID: 34462746; PMCID: PMC8404888; PCT Application Serial No. PCT/US20/61299, Filed on Nov. 19, 2020 an entitled: STRUCTURE-BASED DESIGN OF THERAPEUTICS TARGETING RNA HAIRPIN LOOPS″, Canadian Patent No.: 2,459,347 and Gennemark et al., Sci. Transl. Med. 13, eabe9117 (2021) 12 May 2021) are incorporated herein by reference to disclose and describe aspects, methods and/or materials in connection with the cited publications.

### Illustrative 3D-ASO Products Made by the Processes Disclosed Herein

1: SBD1 for SARS-CoV-2
(SEQ ID NO: 41)
5′-CGTTTAGAGAACAGTTTCT-3′
2: SBD1-T15A for SARS-CoV-2
(SEQ ID NO: 42)
5′-CGTTTAGAGAACAGATTCT-3′
4: PRF3p for inhibiting PRF of SARS-CoV-2
(SEQ ID NO: 43)
5′-GATGTCAAAAGCCCTGTAGTAC-3′
5: PRF3pΔ2 for inhibiting PRF of SARS-CoV-2
(SEQ ID NO: 44)
5′-TGTCAAAAGCCCTGTAGTAC-3′
6: PRF3pΔ1 for inhibiting PRF of SARS-CoV-2
(SEQ ID NO: 45)
5′-ATGTCAAAAGCCCTGTAGTAC-3′
7: DMD1 for DMD exon 51 skipping
(SEQ ID NO: 46)
5′-GATGGCATTTCTAGTCTTG-3′
8: DMD2 for DMD exon 51 skipping
(SEQ ID NO: 47)
5′-AAAψCAψAGGGGAAGATG-3′
9: DMD44-3D1 for DMD exon 44 skipping
(SEQ ID NO: 48)
5′-AAAAAGGTTCAGCTTCTGTTAGCC-3′
10: DMD44-3D2 for DMD exon 44 skipping
(SEQ ID NO: 49)
5′-AAAGGTGAAGCTTCTGTTAGCCAC-3′
11: DMD44-3D3 for DMD exon 44 skipping
(SEQ ID NO: 50)
5′-AAψTAAGAψTATGTTAGCCACTGA-3′
12: DMD44-3D4 for DMD exon 44 skipping
(SEQ ID NO: 51)
5′-AAAGATTCTGTTAGCCACTGATTA-3′
13: DMD45-3D4-1 for DMD exon 45 skipping
(SEQ ID NO: 52)
5′-AAGGGCCGTCCTGGAGTTCCTG-3′
14: DMD45-3D4-2 for DMD exon 45 skipping
(SEQ ID NO: 53)
5′-AAGGGCCATCCTGGAGTTCCTG-3′
15: DMD45-3D3 for DMD exon 45 skipping
(SEQ ID NO: 54)
5′-AAAψGGAAATCCTGGAGTTCCTG-3′
16: DMD45-3D4-1 for DMD exon 45 skipping
(SEQ ID NO: 55)
5′-AAGGGCCGTCCTGGAGTTCCTG-3′
17: DMD45-3D4-2 for DMD exon 45 skipping
(SEQ ID NO: 56)
5′-AAGGGCCATCCTGGAGTTCCTG-3′
18: FAN1SBD1 for regulating human
FAN1 expression
(SEQ ID NO: 57)
5′-CCCAGCCAGAGGGTGGCTAAATTTT-3′
19: FAN1SBD2 for regulating human
FAN1 expression
(SEQ ID NO: 58)
5′-AAAACGGAGGAAATACAATTAG-3′
20: FAN1SBD3 for regulating human
FAN1 expression
(SEQ ID NO: 28)
5′-AATGGGACATATAAGGTTTTTCT-3′
21: FAN1SBD3 for regulating human
FAN1 expression
(SEQ ID NO: 29)
5′-AATGGGACATATAAGGTTTTTCT-3′
22: FAN1SBD4 for regulating human
FAN1 expression
(SEQ ID NO: 30)
5′-AAGTTGCTTATAAAATGT-3′
23: FAN1SBD5 for regulating human
FAN1 expression
(SEQ ID NO: 39)
5′-GGATTCTATACTTCTCAAGTCATTT-3′
24: FAN1SBD6 for regulating human
FAN1 expression
(SEQ ID NO: 40)
AAGAGGGATTTTTAAAAATCT

1. A process for making an antisense oligonucleotide product comprising:
selecting a ribonucleotide target sequence, wherein:
the ribonucleotide target sequence is selected as one present in a naturally occurring ribonucleotide molecule;
the ribonucleotide target sequence is from 8-30 nucleotides in length;
the ribonucleotide target sequence comprises:
a segment of ribonucleotides that forms an RNA loop structure in the naturally occurring ribonucleotide molecule; or
a segment of ribonucleotides that are from 1 to 25 nucleotides distal to a segment of ribonucleotides that forms a hairpin structure in the naturally occurring ribonucleotide molecule; and
constructing the antisense oligonucleotide product, wherein the antisense oligonucleotide product is constructed by:
forming an antisense oligonucleotide product by selecting a plurality of nucleotides such that:
when covalently coupled together the plurality of nucleotides form an antisense oligonucleotide product complementary to the ribonucleotide target sequence, wherein:
(a) one or more nucleotides within the antisense oligonucleotide product are selected to form a bonding interaction with a major-groove RNA structure or a minor-groove RNA structure within the naturally occurring ribonucleotide molecule; and/or
(b) one or more nucleotides within the antisense oligonucleotide product is selected to form a Watson Crick base pairing with the ribonucleotide target sequence and a Hoogsteen base pairing with the naturally occurring ribonucleotide molecule; and/or
(c) nucleotides within the antisense oligonucleotide product do not form base pairing interactions with at least two proximal nucleotides in the ribonucleotide target sequence, wherein the at least two proximal nucleotides in the ribonucleotide target sequence that do not form base pairing interactions with the antisense oligonucleotide product are flanked by a plurality of nucleotides in the ribonucleotide target sequence that form Watson Crick base pairings with the antisense oligonucleotide product; and
forming the antisense oligonucleotide product from the plurality of selected nucleotides so that the antisense oligonucleotide product is made.

2. The process of claim 1, wherein:
the one or more nucleotides within the antisense oligonucleotide product selected to form a bonding interaction with a major-groove or minor groove RNA structure in the naturally occurring ribonucleotide molecule that contributes to the free energy of ASO binding; and/or
the one or more nucleotides within the antisense oligonucleotide product selected to form a Hoogsteen base pairing with the naturally occurring ribonucleotide molecule that contributes to the free energy of ASO binding.

3. The process of claim 1, wherein:
the antisense oligonucleotide product comprises between 13 and 30 nucleotides; or
the antisense oligonucleotide product comprises from 17 to 25 nucleotides.

4. The process of claim 1, wherein:
at least 5, 6, 7, 8, 9 or 10 nucleotides in the antisense oligonucleotide product are selected to form a polynucleotide segment in the antisense oligonucleotide product having 100% complementarity to the ribonucleotide target sequence; and
the antisense oligonucleotide product comprises at least 1, 2, 3, 4, or 5 nucleotides that do not form Watson Crick bonds with the ribonucleotide target sequence such that the antisense oligonucleotide product is not 100% complementary to the ribonucleotide target sequence.

5. The process of claim 4, wherein
the first five terminal 5′ and/or 3′ nucleotides in the antisense oligonucleotide are selected to comprise at least 1 nucleotide that forms: (i) a Hoogsteen base pairing; or (ii) a bonding interaction with a RNA major-groove; or (iii) a bonding interaction with a RNA minor-groove in the naturally occurring ribonucleotide molecule.

6. The antisense oligonucleotide product of claim 2, wherein the antisense oligonucleotide product comprises at least one of:
a pseudouridine;
a phosphorothioate linkage;
a morpholino nucleotide;
a modification of the 2′ sugar position of a ribose moiety;
a 2′-MOE methoxyethyl moiety;
a 2-fluoro moiety; or
a 2′-hydroxy moiety.

7. The process of claim 1, wherein:
the antisense oligonucleotide product consists of from 13 to 30 nucleotides;
at least 5 nucleotides in the antisense oligonucleotide product are selected to form a polynucleotide segment in the antisense oligonucleotide product having 100% complementarity to the ribonucleotide target sequence; and
terminal 5′ and/or 3′ 5 nucleotides in the antisense oligonucleotide product are selected to comprise at least 1 nucleotide that forms a bonding interaction with a major-groove RNA structure or a minor-groove RNA structure in the naturally occurring ribonucleotide molecule.

8. The process of claim 1, wherein:
the antisense oligonucleotide product consists of from 17 to 25 nucleotides;
at least 10 nucleotides in the antisense oligonucleotide product are selected to form a polynucleotide segment in the antisense oligonucleotide product having 100% complementarity to the ribonucleotide target sequence; and
at least 1 nucleotide in the antisense oligonucleotide forms a Watson Crick base pairing with the ribonucleotide target sequence and a Hoogsteen base pairing with the naturally occurring ribonucleotide molecule.

9. The process of claim 1, further comprising combining the antisense oligonucleotide product with a pharmaceutical, liposome or nanoparticle composition.

10. An antisense oligonucleotide product made by the process of claim 1.

11. The antisense oligonucleotide of claim 10, wherein the antisense oligonucleotide product comprises from 13 to 30 nucleotides.

12. The antisense oligonucleotide of claim 10, wherein:
the antisense oligonucleotide product consists of from 17 to 30 nucleotides;
at least 5 nucleotides in the antisense oligonucleotide product form a polynucleotide segment in the antisense oligonucleotide product having 100% complementarity to the ribonucleotide target sequence; and
the first five terminal 5′ and/or 3′ nucleotides in the antisense oligonucleotide comprise at least 1 nucleotide that forms a bonding interaction with a RNA major-groove in the naturally occurring ribonucleotide molecule; or a bonding interaction with a RNA minor-groove in the naturally occurring ribonucleotide molecule.

13. The antisense oligonucleotide of claim 10, wherein:
the antisense oligonucleotide product consists of from 17 to 25 nucleotides;
at least 10 nucleotides in the antisense oligonucleotide product form a polynucleotide segment in the antisense oligonucleotide product having 100% complementarity to the ribonucleotide target sequence; and
at least 1 nucleotide in the antisense oligonucleotide forms a Watson Crick base pairing with the ribonucleotide target sequence and a Hoogsteen base pairing with the naturally occurring ribonucleotide molecule.

14. The antisense oligonucleotide of claim 13, wherein the antisense oligonucleotide comprises at least one of:
a pseudouridine;
a phosphorothioate linkage;
a morpholino nucleotide;
a modification of the 2′ sugar position of a ribose moiety;
a 2′-MOE methoxyethyl moiety;
a 2-fluoro moiety; or
a 2′-hydroxy moiety.

15. The antisense oligonucleotide of claim 10, further comprising at least one of: a pharmaceutical excipient, a liposome or a nanoparticle.

16. The antisense oligonucleotide of claim 10, wherein the target sequence is present in a ribonucleotide expressed by a virus, a bacteria or a fungi.

17. The antisense oligonucleotide of claim 10, wherein the target sequence is present in a ribonucleotide expressed by a human cell.

18. The antisense oligonucleotide of claim 10, wherein the target sequence is present in a ribonucleotide expressed by a human parasite.

19. The antisense oligonucleotide product of claim 16, wherein target sequence is present in a ribonucleotide expressed by:
a human tau gene;
a human beta amyloid gene;
a Covid 19 gene.
a human Duchenne muscular dystrophy gene;
a human FANCD2/FANC1-associated nuclease 1 (KIAA1018) gene; or
a human microRNA (miRNA) gene.

20. The antisense oligonucleotide product of claim 19, wherein the oligonucleotide comprises a sequence:
(SEQ ID NO: 41)
5′-CGTTTAGAGAACAGTTTCT-3′,
(SEQ ID NO: 42)
5′-CGTTTAGAGAACAGATTCT-3′;
(SEQ ID NO: 43)
5′-GATGTCAAAAGCCCTGTAGTAC-3′;
(SEQ ID NO: 44)
5′-TGTCAAAAGCCCTGTAGTAC-3′;
or
(SEQ ID NO: 45)
5′-ATGTCAAAAGCCCTGTAGTAC-3′.
